(19) United States
(12) Patent Application Publication
(10) Pub. No.: US 2026/0345598 A1
(43) Pub. Date: Nov. 5, 2026

(54) LUMATEPERONE AND DERIVATIVES THEREOF FOR MODULATING THE NERVOUS SYSTEM

(71) Applicant: INTRA-CELLULAR THERAPIES, INC., Bedminster, NJ (US)

(72) Inventors: Robert E. DAVIS, San Diego, CA (US); Gretchen SNYDER, New York, NY (US); Sophie DUTHEIL, Stamford, CT (US); Peng LI, New Milford, NJ (US); Emma LEHMANN, New York, NY (US)

(73) Assignee: INTRA-CELLULAR THERAPIES, INC., Bedminster, NJ (US)

(21) Appl. No.: 19/157,641

(22) PCT Filed: Feb. 16, 2024
(86) PCT No.: PCT/US2024/016314

§ 371 Date: Aug. 18, 2025

Related U.S. Application Data
(60) Provisional application No. 63/485,858, filed on Feb. 17, 2023.
(60) Provisional application No. 63/496,661, filed on Apr. 17, 2023.

(51) Int. Cl. A61K 31/4985; A61P 25/00

(52) U.S. Cl. CPC A61K 31/4985; A61P 25/00

(57) ABSTRACT

The present disclosure relates to use of Compounds of Formula (I), Compounds of Formula (II), and related analogs, for enhancing neuritogenesis, enhancing neurite outgrowth, enhancing neural growth, neural connectivity, synaptic density, dendritic spine density, dendritic spine size, and excitatory neurotransmission, in the brain (e.g., in the medial prefrontal cortex), and other methods and uses related thereto.

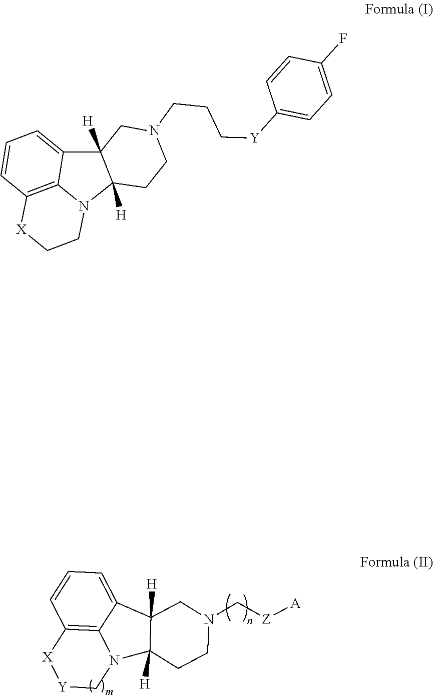

### CROSS-REFERENCE TO RELATED APPLICATIONS

This application is an international application which claims priority to, and the benefit of, U.S. Provisional Application Ser. No. 63/485,858, filed on Feb. 17, 2023, and U.S. Provisional Application Ser. No. 63/496,661, filed on Apr. 17, 2023, the contents of each of which are hereby incorporated by reference in their entireties.

### FIELD

The present disclosure relates to the use of lumateperone, related analogs thereof, and other octahydro-1H-pyrido[3′,4′: 4,5]pyrrolo[1,2,3-de]quinoxalines, for enhancing neuritogenesis, enhancing neurite outgrowth, enhancing neural growth, neural connectivity, synaptic density, dendritic spine density, dendritic spine size, and excitatory neurotransmission, in the brain (e.g., in the prefrontal cortex).

### BACKGROUND

Lumateperone, having the formula:

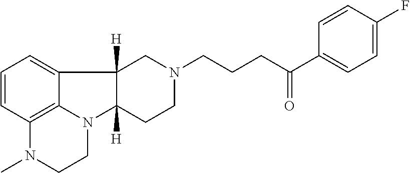 [SMILES: CN1CCN2c3c(cccc31)[C@@H]1CN(CCCC(=O)c3ccc(F)cc3)CC[C@@H]12]

is a novel therapeutic agent with potent (Ki=0.5 nM) 5-HT_{2A} receptor antagonism, activity as a mesolimbic/mesocortical-selective dopamine receptor protein phosphorylation modulator consistent with presynaptic D2 receptor partial agonism and postsynaptic D2 receptor antagonism (Ki=32 nM) in vivo, high D1 receptor affinity (Ki=52 nM), and inhibition of the serotonin transporter (SERT) (Ki=26-62 nM, using different assays for SERT activity).

Lumateperone is principally known to be useful for the treatment of positive and negative symptoms of schizophrenia. At dopamine D2 receptors, it has dual properties and acts as both a post-synaptic antagonist and a pre-synaptic partial agonist. It also stimulates phosphorylation of glutamatergic NMDA NR2B (GluN2B) receptors in a mesolimbic specific manner. It is believed that this regional selectivity in the brain areas thought to mediate the efficacy of antipsychotic drugs, together with the serotonergic, glutamatergic, and dopaminergic interactions, may result in antipsychotic efficacy for positive, negative, affective and cognitive symptoms associated with schizophrenia.

Lumateperone also exhibits serotonin reuptake inhibition, providing antidepressant activity for the treatment of schizoaffective disorder, co-morbid depression, and/or as a stand-alone treatment for bipolar depression or major depressive disorder. Lumateperone is also useful for the treatment of bipolar disorder and other psychiatric and neurodegenerative disorders, particularly behavioral disturbances associated with dementia, autism and other CNS diseases. These features may be able to improve the quality of life of patients with schizophrenia and enhance social function to allow them to more fully integrate into their families and their workplace.

Lumateperone displays differential dose-dependent effects, selectively targeting the 5-HT_{2A} receptor at low doses, while progressively interacting with the D2 receptor at higher doses. As a result, at lower doses, it is useful in treating sleep, aggression and agitation. At a higher dose, it can treat acute exacerbated and residual schizophrenia, bipolar disorders, and mood disorders.

The very unique pharmacological profile of lumateperone results in possessing some properties of both an atypical antipsychotic agent (APD) and a selective serotonin reuptake inhibitor (SSRI). It has been found to enhance glutamatergic neurotransmission by effects on both NMDA and AMPA receptor conductance in rat medial prefrontal cortex (mPFC) slices. The actions of lumateperone are consistent with the effects of other rapid-acting antidepressant therapies, including the combined use of olanzapine (a D2-receptor antagonist APD) with fluoxetine (an SSRI) and of ketamine.

Lumateperone has been approved in the United States for the treatment of schizophrenia and bipolar depression, and is in development as a treatment for schizophrenia, bipolar depression and agitation in dementia, including Alzheimer's Disease.

Lumateperone and related compounds have been disclosed in U.S. Pat. Nos. 6,552,017; 7,071,186; 7,183,282; and U.S. RE39,680, for the treatment of disorders associated with 5-HT_{2A} receptor modulation such as anxiety, depression, psychosis, and schizophrenia. U.S. Pat. Nos. 8,598,119, 9,616,061, and 10,117,867, each incorporated herein by reference, disclose the use of lumatepcrone for the treatment of depression, schizophrenia and sleep disorders. U.S. Pat. Nos. 11,053,245 and 11,124,514, each incorporated herein by reference, disclose the use of lumateperone for the treatment of a combination of psychosis and depressive disorders as well as sleep, depressive and/or mood disorders in patients with psychosis or Parkinson's disease and for the treatment or prophylaxis of disorders associated with dementia, particularly behavioral or mood disturbances such as agitation, irritation, aggressive/assaultive behavior, anger, physical or emotional outbursts and psychosis and sleep disorders associated with dementia.

U.S. Pat. No. 8,648,077, incorporated herein by reference, discloses methods of preparing toluenesulfonic acid addition salt crystals of particular substituted heterocycle fused gamma-carbolines, e.g., toluenesulfonic acid addition salt of 4-((6bR,10aS)-3-methyl-2,3,6b,9,10,10a-hexahydro-1H-pyrido[3′,4′: 4,5]pyrrolo[1,2,3-de]quinoxalin-8 (7H)-yl)-1-(4-fluorophenyl)-1-butanone.

U.S. Pat. Nos. 10,077,267, 10,688,097, and US 2021/0008065, each incorporated herein by reference, deuterated forms of lumateperone and related compounds have been shown to have improved metabolic stability.

US 2021/0060009 discloses the use of lumateperone, and its deuterated analogs, for the acute treatment of anxiety and depression (e.g., for the treatment of acute anxiety and acute depression). Conventional antidepressants often take weeks or months to achieve their full effects, so are generally not effective for acute treatment of depression. This delayed onset of action increases the risk for suicidal behavior. Unlike traditional antidepressants, which predominantly operate within the monoamine neurotransmitter sphere (i.e., serotonin, norepinephrine, and dopamine), ketamine is a selective NMDA receptor antagonist, acting through separate systems unrelated directly to the monoamines, and this is a major reason for its much more rapid antidepressant effect compared to the traditional antidepressants. Ketamine directly antagonizes extrasynaptic glutamatergic NMDA receptors, which also indirectly results in activation of AMPA-type glutamate receptors. The downstream effects involve the brain-derived neurotrophic factor (BDNF) and mTOR (e.g., mTORC1) kinase pathways (signal transduction pathways). Lumateperone has been found to have very similar activity on NMDA/AMPA receptors and downstream BDNF and mTOR signaling as ketamine, and thus, it may provide a rapid antidepressant effect similar to that of ketamine. See, e.g., Dutheil et al., J. Neurosci., 43 (5): 863-877 (2023); Titulacr et al., Eur. Neuropsychopharm., 62:22-35 (2022).

Serotonin, also known as 5-hydroxytryptamine (5-HT), is a neurotransmitter widely distributed in the brain. Drugs that directly or indirectly target 5-HT, such 5-HT receptor agonists and selective serotonin reuptake inhibitors, are widely used in psychiatry, finding use in the treatment of numerous mood disorders as well as in the treatment of psychosis. However, strong 5-HT agonists tend to cause hallucinations, which is a dangerous side effect. There have been studies suggesting that the classic hallucinogenic psychedelic serotonin agonists, such as LSD (D-lysergic acid diethylamide) and psilocybin (via its active metabolite psilocin) could be very effective in the treatment of numerous neuropsychiatric disorders, especially depression, but these drugs are not feasible in practice because of their hallucinogenic side effects. Therefore, there has been an effort to develop novel compounds having a pharmacological profile similar to the hallucinogenic psychedelics, but without the hallucinations.

Psilocybin is a serotonergic psychedelic derived from the hallucinogenic mushrooms of the genus Psilocybe. Psilocybin itself is inactive, but it is a pro-drug of the active compound psilocin, formed via a rapid enzymatic dephosphorylation:

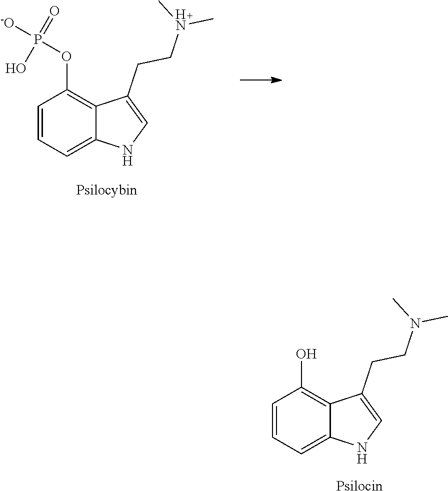

Psilocin is most prominently a strong agonist of the 5-HT_{2A} receptor, with lesser activity at other serotonin receptors. Psilocin has similar mind-altering effects to LSD (lysergic acid diethylamide), mescaline, ibogaine and DMT (N,N-dimethyltryptamine), including euphoria, visual and auditory hallucinations, changes in perception, distorted sense of time, and perceived spiritual experiences. In addition, it can cause nausea and panic attacks (e.g., “bad trips”). The effects can last for 2 to 6 hours. Psilocybin has been proposed to have great untapped therapeutic potential, such as in treating depression, but it has been hampered by the widespread classification of psilocin and psilocybin as controlled substances.

Psilocybin has recently been studied in mice for its effect of promoting the growth of dendritic spines in cells of the frontal cortex in mice. Shao et al., Neuron, 109 (16): 2535-2544 (2021). Neurons communicate with each other primarily by transfer of signals across synapses. Synapses are junctions between the cell membranes of two neurons (or between a neuron and a muscle cells) in which a signal is relayed by the release of neurotransmitter molecules on the presynaptic side of the synapse, and binding of those neurotransmitter molecules to receptors on the post-synaptic side of the synapse. Most neurons are arranged with a cell body having numerous short, branching extensions called dendrites, and a single long extension called an axon. Dendrites have numerous smaller surface spikes called dendritic spines, at the ends of which are synapses connecting the nerve cell to other nerve cells. These dendritic synapses serve as a way for the neuron to receive messages from other neurons, while the axon is terminated by synapses that are used to transmit a message to other neurons (or muscle cells). Dendrites and dendritic spines, as well as axons, are therefore essential to the ability of nerve cells to communicate with each other.

The vast majority of neurons are multipolar neurons, meaning that they have a cell body with numerous process extension, called neurites. Dendrites and axons are both considered types of neurites. Particularly when it comes to cell cultures of neurons, or immature or developing neurons in vivo, the term neurite is often used because it can be difficult to distinguish between a growing axon and a growing dendrite, before cellular differentiation is complete. Thus, the sprouting and subsequent growth of neurites is referred to as neuritogenesis (or neurite outgrowth), and this includes both the growth of dendrites and the growth of axons, particularly in an immature neuronal cell. The initial sprouting of a neurite is a three-step process: first the original round shape of the cell is broken down to make a bud, then the bud is transformed into a neurite, then the neurite is transformed into an axon or dendrite.

Neuritogenesis can be studied using different qualitative or quantitative measures, such as based on histological or immunochemical staining of cell cultures. Measures can include cell size or length, total number of neurites per cell, length of the neurites (individually and/or in total for a neuron), the number of branch points on the neurites, the number of neurite roots, the number of neurite nodes, the total number of neurite extremities, and the total length of neurons with or without its branches. This can all be measured using automated or semi-automated image analysis techniques. Total neurite arborization can also be measured or estimated, such as, using a Sholl Analysis. Several assay methods have been disclosed for neuritogenesis analysis, which may be used for testing the compounds disclosed herein. See, e.g., Li, S. et al., “Evaluation of Chemical Compounds that Inhibit Neurite Outgrowth in iPSC-derived Human Neurons,” Neurotoxicology 83:137-145 (2021); Li, Z. et al., “High-throughput neurite outgrowth assay using GFP-labeled iPSC-derived neurons,” Curr Protoc. 2 (9): e542 (2022); Duchemin, C. et al., “Compounds with different pharmacological profiles enhance the neurite outgrowth in Human iPSC derived neurons (Poster), iForum 2016 (available from Neurofit.com).

Several neurological disorders, including depression, have been associated with synaptic atrophy in certain regions of the brain and resultant loss of connectivity between cells. That is, the interconnections between neurons at dendritic synapses is lost in the impaired or damaged brain. For example, when imaging patients with major depressive disorder (MDD), it has been frequently found that they suffer from measurable atrophy in the dorsal prefrontal cortex (PFC). In addition, post-mortem observations of MDD patients have confirmed what pre-clinical studies in rodent models of depression had previously highlighted: protein levels involved in the mammalian target of rapamycin (mTOR) signaling pathway are decreased in the PFC. The importance of these alterations has been reinforced by the discovery that stress causes atrophy of stress-vulnerable hippocampal neurons and pyramidal neurons in the medial PFC (mPFC), concomitant with decreases in AMPA receptors, mTORC1 signaling, and brain-derived neurotrophic factor (BDNF).

It has been suggested that some drugs with rapid antidepressant action, such as ketamine and psilocybin, may exert their effects in part by promoting the growth of new dendritic spines, and from these, new synaptic connections, in the brain (so called “structural remodeling” or “synaptic plasticity” of the brain). Such antidepressants have been suggested to operate, at least in part, by inducing transient increases in mTORC1 activity in the brain, resulting in increased synaptic spine density in the mPFC. Neuroimaging studies have suggested improved functional connectivity in the right lateral PFC of patients with MDD after ketamine treatment.

Shao et al. used an in vivo rodent model to demonstrate that a single dose of psilocybin results in a measurable increase in dendritic spine density, as well as increased spine head width, within one day. Shao further showed that the measurable changes in dendritic spine density persist for at least a month following the single dose of psilocybin. Shao specifically demonstrated that the increased dendritic spine density was due to increased formation rate, rather than reduced elimination rate, in both male and female mice. Interestingly, pre-treatment with the 5-HT_{2A} receptor antagonist ketanserin, at a dose that blocked about 30% of the 5-HT_{2A} receptors, was sufficient to block the psilocybin-induced head twitch response (a functional measure of serotonin receptor activity) but did not eliminate the psilocybin-induced growth of dendritic spines. Shao et al. did not rule out that the remaining receptors unaffected by ketanserin might be involved in the dendritic remodeling. Finally, Shao also showed that these structural effects in medial prefrontal cortex neurons were also associated with increased excitatory neurotransmission in the medial prefrontal cortex (miniature excitatory post-synaptic currents, mEPSCs).

Psilocybin is however a strong hallucinogen. It remains to be seen whether the hallucinogenic effects of psilocybin can be dissociated from the structural remodeling effect by modification of the psilocybin structure. This is particularly important for psychosis patients (e.g., schizophrenia or related disorders) who may otherwise be in need of therapies which provide neural structural remodeling, but for whom even mild hallucinogenic side effects could severely exacerbate the psychotic disorder. Similarly, patients with dementia are at an increased risk of severe adverse events resulting from drug-induced hallucination because of their reduced ability to recognize the side effects. There are many patients for whom hallucinogenic psychedelic therapy is contraindicated, so there is a need for drugs without hallucinogenic side effects.

There is thus a need for new drug molecules that may provide the serotonin-receptor mediated structural remodeling effects recently shown for psilocybin, but without the hallucinogenic side effects.

### BRIEF SUMMARY

While psilocybin is a strong agonist of the 5-HT_{2A} receptor, lumateperone is a strong antagonist of the 5-HT_{2A} receptor. Shao et al., suggested that both psilocybin's hallucinogenic effects and its effects on neural structural remodeling are the result of 5-HT_{2A} receptor agonism. Because lumateperone does not have serotonin receptor agonist activity, it is non-hallucinogenic. However, it is believed that lumateperone may have the same neural structural remodeling effect in the brain as psilocybin, but instead mediated via enhancement of mTOR signaling (whether psilocybin similarly has a direct or indirect effect on mTOR signaling is not yet known, but it is possible that psilocybin agonism of 5-HT_{2A} receptors on prefrontal cortex neurons activates mTOR signaling). Specifically, without being bound by theory, it is believed that by activating D1 receptors in the brain (e.g., in mPFC and/or amygdala), lumateperone leads to FYN kinase-mediated phosphorylation of the GluN2B subunit of NMDA receptor and thereby enhanced NMDA-mediated neurotransmission in prefrontal neurons. As a result, glutamate release is increased which enhances AMPA mediated currents, BDNF release, and mTORC1 activation. Increased synaptogenesis has been associated with enhanced mTORC1 signaling in the PFC. Recent experiments have shown that the acute administration of lumateperone stimulates PFC mTORC1-related signaling pathways, as well as some important genes coding for neurotrophic factors, such as BDNF and VEGF. Consistent with this theory, we have surprisingly found that lumateperone may induce structural remodeling in the brain, such as enhancing neural growth, neural connectivity, synaptic density, dendritic spine density, dendritic spine size, and excitatory neurotransmission, such as in the medial prefrontal cortex region. Analogs of lumateperone and other octahydro-1H-pyrido[3′,4′: 4,5]pyrrolo[1,2,3-de]quinoxalines are expected to have similar properties.

In addition, several new compounds (octahydro-1H-pyrido[3′,4′: 4,5]pyrrolo[1,2,3-de]quinoxalines) have recently been disclosed in International Application No. PCT/US2023/86562, incorporated by reference herein in its entirety, which are selective biased ligands of the serotonin 5-HT_{2A} receptor. These receptors can function either through the classic G_{q}-coupled signaling cascade, or through an alternative beta-arrestin mediated signaling cascade. It has been suggested that the G_{q}-mediated signaling cascade is responsible for the hallucinogenic effects of the traditional psychedelics, and that ligands which are biased towards beta-arrestin recruitment may provide the therapeutic benefits of the psychedelics, such as antidepressant action, without hallucinogenic side effects. The compounds disclosed in PCT/US2023/86562 are functionally biased towards 5-HT_{2A} receptor beta-arrestin signaling. Many of the compounds disclosed are either partial or full agonists of beta-arrestin signaling, but either inactive or antagonistic of G_{q} signaling. Other of these compounds are antagonistic at both pathways but with a functional bias towards beta-arrestin signaling.

The present disclosure thus provides a method for enhancing neural growth, enhancing neural connectivity, increasing synaptic density, increasing dendritic spine density, increasing dendritic spine size, and/or increasing excitatory neurotransmission, in the brain of a subject in need thereof, e.g., in the medial prefrontal cortex region of the brain, the method comprising administering an effective amount of lumateperone, related analogs thereof, deuterated analogs thereof, and other octahydro-1H-pyrido[3′,4′: 4,5]pyrrolo[1,2,3-de]quinoxalines, in free, or pharmaceutically acceptable salt form, to the subject. In some embodiments of this aspect, the enhanced neural growth, enhanced neural connectivity, increased synaptic density, increased dendritic spine density, increased dendritic spine size, and/or increased excitatory neurotransmission is characterized by or caused by enhanced neuritogenesis and/or enhanced neurite outgrowth (e.g., characterized by increases in the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization), and/or is characterized by or associated with changes in synaptic neurotransmitter receptor or neurotransmitter transporter density.

The present disclosure further provides a method for enhancing neuritogenesis and/or enhancing neurite outgrowth, in the brain of a subject in need thereof, e.g., in the medial prefrontal cortex region of the brain, the method comprising administering an effective amount of lumateperone, related analogs thereof, deuterated analogs thereof, and other octahydro-1H-pyrido[3′,4′: 4,5]pyrrolo[1,2,3-de]quinoxalines, in free, or pharmaceutically acceptable salt form, to the subject. In some embodiments of this aspect, the enhanced neuritogenesis and/or enhanced neurite outgrowth is characterized by increases in the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization. In some embodiments, the enhanced neuritogenesis and/or enhanced neurite outgrowth is also associated with changes in synaptic neurotransmitter receptor or neurotransmitter transporter density.

The present disclosure further provides a method for enhancing neural growth, enhancing neuritogenesis, enhancing neurite outgrowth, enhancing neural connectivity, increasing synaptic density, increasing dendritic spine density, increasing dendritic spine size, and/or increasing excitatory neurotransmission, in a neural cell or neural tissue (e.g., a cell culture), in vitro or in vivo, the method comprising the step of contacting the neural cell or neural tissue with an effective amount of lumateperone, related analogs thereof, deuterated analogs thereof, and other octahydro-1H-pyrido[3′,4′: 4,5]pyrrolo[1,2,3-de]quinoxalines, in free, or pharmaceutically acceptable salt form.

### DETAILED DESCRIPTION

In a first aspect, the present disclosure provides a method for enhancing neural growth, enhancing neural connectivity, increasing synaptic density, increasing dendritic spine density, increasing dendritic spine size, and/or increasing excitatory neurotransmission, in the brain of a subject in need thereof, e.g., in the prefrontal cortex region of the brain, the method comprising the step of administering an effective amount of a Compound of Formula I or a Compound of Formula II, as described herein below.

Thus, in a first embodiment of the first aspect, the present disclosure provides a method (Method 1) for enhancing neural growth, enhancing neural connectivity, increasing synaptic density, increasing dendritic spine density, increasing dendritic spine size, and/or increasing excitatory neurotransmission, in the brain of a subject in need thereof, e.g., in the prefrontal cortex region of the brain, the method comprising the step of administering an effective amount of a Compound of Formula I (Compound I):

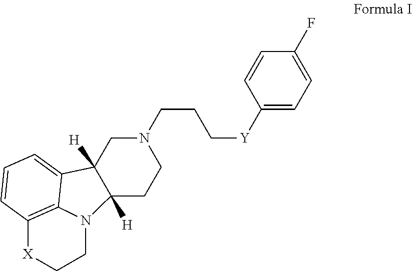

wherein X is selected from —O—, —S—, —N(H), and —N(CH₃)—, and Y is selected from —O—, —C(O)—, —CH(OH)—, and —CH(OCH₃);

or a deuterated analog thereof, in free, or pharmaceutically acceptable salt form, to the subject.

In further embodiments of the first embodiment of the first aspect, the present disclosure provides:
1.1. Method 1, wherein in the Compound of Formula I, X is —O— or —S—;
1.2. Method 1, wherein in the Compound of Formula I, X is —N(H) or —N(CH₃)—;
1.3. Method 1, wherein in the Compound of Formula I, X is —N(CH₃)—;
1.4. Method 1, or any of 1.1-1.3, wherein in the Compound of Formula I, Y is —O—;
1.5. Method 1, or any of 1.1-1.3, wherein in the Compound of Formula I, Y is —C(O)—;
1.6. Method 1, or any of 1.1-1.3, wherein in the Compound of Formula I, Y is —CH(OH)—;
1.7 Method 1, or any of 1.1-1.6, wherein the Compound of Formula I is selected from the group consisting of:

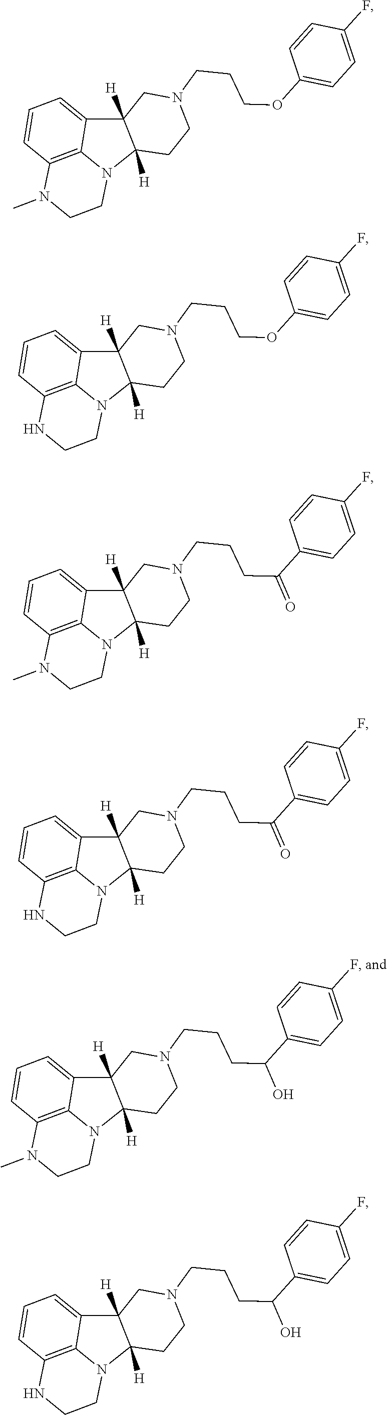

or a deuterated analog thereof;
1.8. Method 1, or any of 1.1-1.7, wherein the Compound of Formula I is selected from:

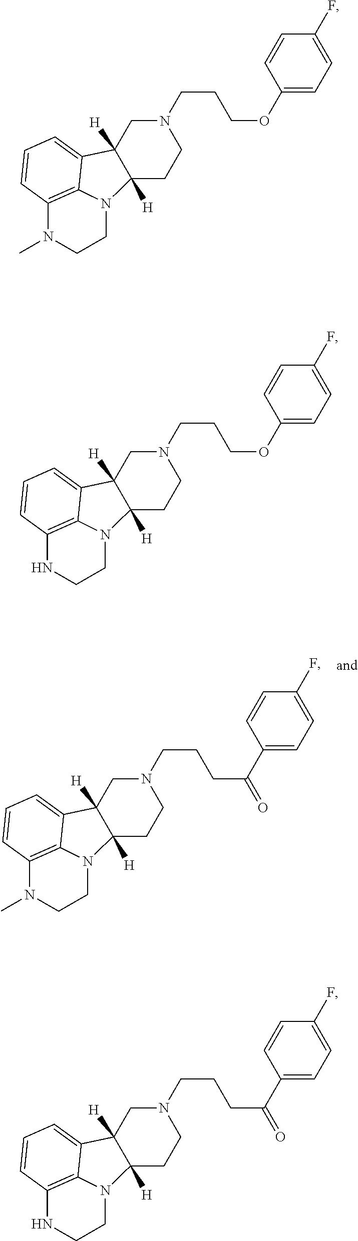 [SMILES: CN1CCN2c3c(cccc31)[C@@H]1CN(CCCOc3ccc(F)cc3)CC[C@@H]12 ; Fc1ccc(OCCCN2CC[C@H]3[C@@H](C2)c2cccc4c2N3CCN4)cc1 ; CN1CCN2c3c(cccc31)[C@@H]1CN(CCCC(=O)c3ccc(F)cc3)CC[C@@H]12 ; O=C(CCCN1CC[C@H]2[C@@H](C1)c1cccc3c1N2CCN3)c1ccc(F)cc1]

or a deuterated analog thereof;
1.9. Method 1, or any of 1.1-1.7, wherein the Compound of Formula I is selected from:

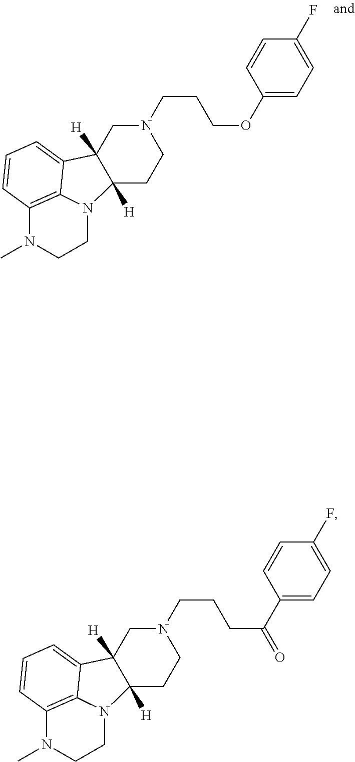 [SMILES: CN1CCN2c3c(cccc31)[C@@H]1CN(CCCOc3ccc(F)cc3)CC[C@@H]12 ; CN1CCN2c3c(cccc31)[C@@H]1CN(CCCC(=O)c3ccc(F)cc3)CC[C@@H]12]

or a deuterated analog thereof;
1.10. Method 1, or any of 1.1-1.7, wherein the Compound of Formula I is:

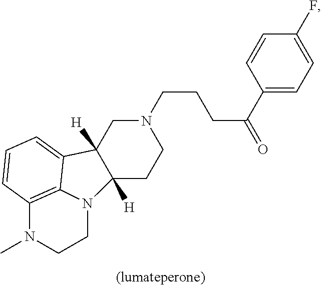 [SMILES: CN1CCN2c3c(cccc31)[C@@H]1CN(CCCC(=O)c3ccc(F)cc3)CC[C@@H]12]

or a deuterated analog thereof;
1.11. Method 1, or any of 1.1-1.10, wherein the Compound of Formula I or deuterated analog thereof is in the form of a free base;
1.12. Method 1, or any of 1.1-1.11, wherein the Compound of Formula I or deuterated analog thereof is in the form of a pharmaceutically acceptable salt;
1.13. Method 1.12, wherein the pharmaceutically acceptable salt is a toluenesulfonic acid addition salt (e.g., a mono-tosylate salt or a bis-tosylate salt);
1.14. Method 1 or any of 1.1-1.13, wherein the Compound of Formula I or deuterated analog thereof is non-deuterated lumateperone, i.e., having the following structure:

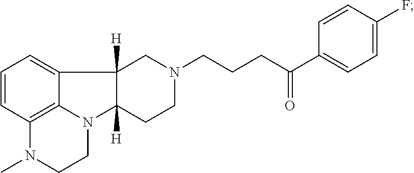 [SMILES: CN1CCN2c3c(cccc31)[C@@H]1CN(CCCC(=O)c3ccc(F)cc3)CC[C@@H]12]

1.15. Method 1 or any of 1.1-1.14, wherein the Compound of Formula I or salt thereof is in deuterated form, e.g., wherein the deuterium: protium ratio for at least one specified carbon-bound hydrogen atom is significantly higher, e.g., at least 2×, for example at least 10× higher, than the natural isotope ratios;
1.16. Method 1.15, wherein the deuterated analog of the Compound of Formula I is selected from

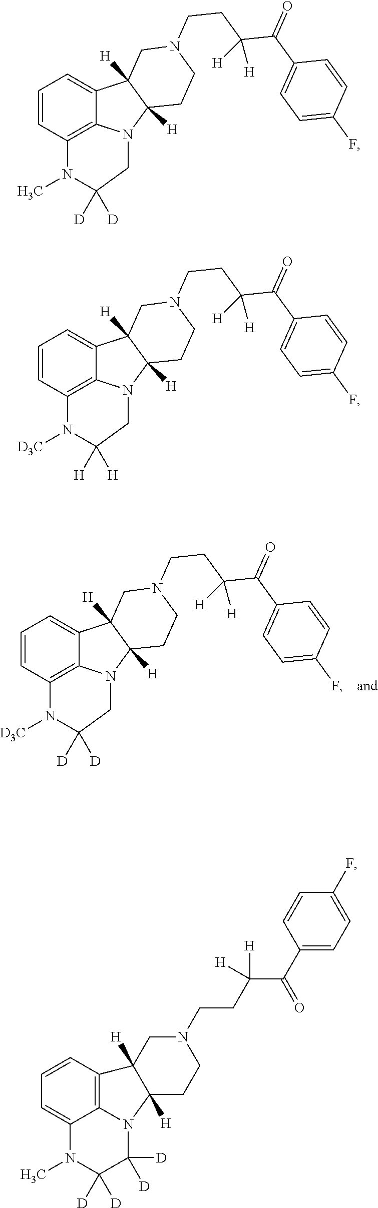 [SMILES: [2H]C1([2H])CN2c3c(cccc3N1C)[C@@H]1CN(CCCC(=O)c3ccc(F)cc3)CC[C@@H]12 ; [2H]C([2H])([2H])N1CCN2c3c(cccc31)[C@@H]1CN(CCCC(=O)c3ccc(F)cc3)CC[C@@H]12 ; [2H]C([2H])([2H])N1c2cccc3c2N(CC1([2H])[2H])[C@H]1CCN(CCCC(=O)c2ccc(F)cc2)C[C@@H]31 ; "and" ; [2H]C1([2H])N(C)c2cccc3c2N([C@H]2CCN(CCCC(=O)c4ccc(F)cc4)C[C@@H]32)C1([2H])[2H]]

in free or pharmaceutically acceptable salt form;
wherein D represents a hydrogen position with substantially greater than natural deuterium incorporation (i.e., substantially greater than 0.0156%), e.g., greater than 60%, or greater than 70%, or greater than 80%, or greater than 90% or greater than 95%, or greater than 96%, or greater than 97%, or greater than 98%, or greater than 99%, in free or pharmaceutically acceptable salt form, e.g., tosylate salt form;
1.17. Any foregoing method, wherein the method provides enhanced neural growth in the brain of the subject (e.g., in the prefrontal cortex region of the brain);
1.18. Any foregoing method, wherein the method provides enhanced neural connectivity, in the brain of the subject (e.g., in the prefrontal cortex region of the brain);
1.19. Any foregoing method, wherein the method provides increased synaptic density, in the brain of the subject (e.g., in the prefrontal cortex region of the brain);
1.20. Any foregoing method, wherein the method provides increased dendritic spine density, in the brain of the subject (e.g., in the prefrontal cortex region of the brain);
1.21. Any foregoing method, wherein the method provides increased dendritic spine size (e.g., increased width of spine heads and/or increased spine protrusion lengths), in the brain of the subject (e.g., in the prefrontal cortex region of the brain);
1.22. Any foregoing method, wherein the method provides increased excitatory neurotransmission (e.g., enhanced glutamatergic transmission or increased rate of mEPSCs), in the brain of the subject (e.g., in the prefrontal cortex region of the brain);
1.23. Method 1, or any of 1.1-1.22, wherein the method provides the enhanced neural growth, enhanced neural connectivity, increased synaptic density, increased dendritic spine density, increased dendritic spine size, and/or increased excitatory neurotransmission, in the brain of the subject (e.g., in the medial prefrontal cortex region of the brain) within less than 4 weeks of the initiation of administration of the Compound of Formula I or deuterated analog thereof, e.g., less than 3 weeks, less than 2 weeks, less than 1 week, less than 6 days, less than 5 days, less than 4 days, less than 3 days, or less than 2 days, after the initiation of treatment with the Compound of Formula I or deuterated analog thereof;
1.24. Method 1, or any of 1.1-1.23, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the Compound of Formula I or deuterated analog thereof, e.g., for at least 3 weeks, or at least 4 weeks, or at least 2 months, or at least 3 months;
1.25. Method 1, or any of 1.1-1.24, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the Compound of Formula I or deuterated analog thereof, e.g., for at least 3 weeks, or at least 4 weeks, or at least 2 months, or at least 3 months, such as by measured by in vivo imaging (e.g., MRI);
1.26. Method 1, or any of 1.1-1.25, wherein the enhanced neural growth, enhanced neural connectivity, increased synaptic density, increased dendritic spine density, increased dendritic spine size, and/or increased excitatory neurotransmission is characterized by or caused by enhanced neuritogenesis and/or enhanced neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
1.27. Method 1, or any of 1.1-1.26, wherein the method enhances neuritogenesis and/or enhances neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
1.28. Method 1.26 or 1.27, wherein the enhanced neuritogenesis or enhanced neurite outgrowth is characterized by increases in one or more of: the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization (e.g., measured or estimated using a Sholl Analysis);
1.29. Any foregoing method, wherein the method does not cause hallucinogenic side effects;
1.30. Any foregoing method, wherein the Compound of Formula I or deuterated analog thereof is administered in a daily dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base;
1.31. Any foregoing method, wherein the Compound of Formula I or deuterated analog thereof is administered in a dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base, at a frequency of every other day, or every two days, or every three days, or every four days, or every five days, or every six days, or every seven days;
1.32. Method 1, or any of 1.1-1.31, wherein the Compound of Formula I or deuterated analog thereof is administered as a unit dosage form for oral administration (e.g., enteral), for example, a tablet or capsule;
1.33. Method 1.32, wherein the unit dosage for oral administration (e.g., enteral), for example a tablet or capsule, comprises the Compound of Formula I or deuterated analog thereof in an amount equivalent 1 to 100 mg of free base, e.g., 1 to 75 mg, or 1 to 60 mg, or 1 to 40 mg, or 1 to 20 mg, or 1 to 10 mg, of free base, and a pharmaceutically acceptable diluent or carrier;
1.34. Method 1, or any of 1.1-1.31, wherein the Compound of Formula I or deuterated analog thereof is administered as a unit dosage for subcutaneous or transmucosal administration, e.g., a sublingual or buccal orally disintegrating tablet or film;
1.35. Method 1.34, wherein the unit dosage for subcutaneous or transmucosal administration, e.g., a sublingual or buccal orally disintegrating tablet or film, comprises the Compound of Formula I or deuterated analog thereof in an amount equivalent to 0.5 to 30 mg of free base, e.g., 1-10 mg of free base, and a pharmaceutically acceptable diluent or carrier;
1.36. Method 1, or any of 1.1-1.31, wherein the Compound of Formula I or deuterated analog thereof is administered as a long-acting injectable (LAI) composition, e.g., for intramuscular or subcutaneous injection;
1.37. Method 1.36, wherein the dose of the LAI composition is sufficient to provide the equivalent of a daily dose of 1 to 100 mg of free base of the Compound I or deuterated analog thereof, e.g., 1 to 75 mg, or 1 to 60 mg, or 1 to 40 mg, or 1 to 20 mg, or 1 to 10 mg, of free base, released over a period of time ranging from about 1 week to about 3 months, e.g., about 1 week to about 8 weeks, or about 1 week to about 6 weeks, or about 1 week to about 4 weeks, or about 1 week to about 3 weeks, or about 1 week to about 2 weeks;
1.38. Method 1.36 or 1.37, wherein the LAI composition comprises the Compound of Formula I or deuterated analog thereof dissolved, dispersed, suspended, or encapsulated in a polymeric matrix;
1.39. Method 1.38, wherein the polymeric matrix comprises one or more biocompatible and biodegradable polymers as defined herein, e.g., poly(hydroxycarboxylic acids), poly(amino acids), cellulose polymers, modified cellulose polymers, polyamides, and polyesters;
1.40. Method 1.39, wherein the one or more polymers comprises polylactic acid, polyglycolic acid, polycitric acid, polymalic acid, poly-beta-hydroxybutyric acid, poly(lactic acid-glycolic acid) copolymer, 2-hydroxybutyric acid-glycolic acid copolymer, polylactic acid-polyethylene glycol copolymer, polyglycolic acid-polyethylene glycol copolymer, poly(alkyl alpha-cyanoacrylate) such as poly(butyl cyanoacrylate) or poly(2-octyl cyanoacrylate), poly(ortho ester), polycarbonate, polyortho-carbonate, a polyamino acid, (for example poly-gamma.-L-alanine, poly-.gamma.-benzyl-L-glutamic acid or poly-y-methyl-L-glutamic acid), and/or hyaluronic acid ester;
1.41. Method 1.39, wherein the one or more polymers comprises polylactic acid, polyglycolic acid, polycitric acid, polymalic acid, or a poly(lactic acid-glycolic acid) copolymer;
1.42. Method 1.39, wherein the one or more polymers comprises a poly(lactic acid-glycolic acid) copolymer, e.g., poly-d,l-lactide-co-glycolide;
1.43. Any foregoing method, wherein the subject is an animal;
1.44. Any foregoing method, wherein the subject is a human (e.g., a patient suffering from a neuropsychiatric disorder);
1.45. Method 1.44, wherein the subject is a patient suffering from anxiety or depression, e.g., bipolar depression, major depressive disorder (MDD), post-traumatic stress disorder, or treatment-resistant depression;
1.46. Method 1.45, wherein the subject is a patient suffering from treatment resistant depression (e.g., depression which has not responded to treatment with an antidepressant agent selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof);
1.47. Method 1.45, wherein the subject is a patient suffering from bipolar depression or major depressive disorder;
1.48. Any foregoing method, wherein the method further comprises the concurrent administration of an anti-depressant agent (e.g., selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof), e.g., administered simultaneously, separately or sequentially;
1.49. Any foregoing method, wherein the method further comprises the concurrent administration of an NMDA receptor antagonist, for example, selected from ketamine (e.g., S-ketamine and/or R-ketamine), hydroxynorketamine, memantine, dextromethorphan, dextroallorphan, dextrorphan, amantadine, and agmatine, or any combination thereof, e.g., administered simultaneously, separately or sequentially;
1.50. Any foregoing method, wherein the method further comprises the concurrent administration of a NMDA receptor allosteric modulator, e.g., a NMDA receptor glycine-site modulator, such as rapastinel, nebostinel, apimostinel, D-cycloserine, or any combination thereof, e.g., administered simultaneously, separately or sequentially;
1.51. Any foregoing method, wherein the subject has previously been treated with but has not responded to, or has not responded adequately to, or who suffers undesirable side effects from, treatment with another antidepressant agent, for example, any one or more of a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor, or a serotonin receptor antagonist;
1.52. Any foregoing method, wherein the Compound of Formula I or deuterated analog thereof is administered as monotherapy, e.g., it is not administered concurrently or in conjunction with an anti-depressant, anti-psychotic, or anti-anxiety agent;
1.53. Any foregoing method, wherein the Compound of Formula I or deuterated analog thereof is administered without the direct supervision of a health care professional (e.g., the compound is self-administered by the subject (e.g., patient));
1.54. Any foregoing method, wherein the method does not comprise supervision or observation of the subject (e.g., patient) by a health care professional during or after (e.g., within 2 hours after) administration of a dose of the Compound of Formula I or deuterated analog thereof;
1.55. Any foregoing method, wherein the method does not put the subject (e.g., patient) at risk for sedation, dissociation, abuse, misuse, or suicidal ideation;
1.56. Any foregoing method, wherein the method does not result in hypertension (e.g., systolic and/or diastolic hypertension) within four hours after administration of a dose of the Compound of Formula I or deuterated analog thereof, e.g., an increase of more than 10 mm Hg, or more than 20 mm Hg, or more than 30 mm Hg, or more than 40 mm Hg, in systolic and/or diastolic blood pressure within 30 minutes to 4 hours after said dose;
1.57. Any foregoing method, wherein the method does not result in cognitive decline in the subject (e.g., patient);
1.58. Any foregoing method, wherein the subject (e.g., patient) has been diagnosed with or is at risk of aneurysmal vascular disease (e.g., thoracic aorta, abdominal aorta, intracranial, or peripheral arterial aneurysms), arteriovenous malformation or intracerebral hemorrhage;
1.59. Any foregoing method, wherein the subject (e.g., patient) is under concurrent treatment with an oral antidepressant selected from duloxetine, escitalopram, sertraline, or venlafaxine;
1.60. Any foregoing method, wherein the subject (e.g., patient) is not under concurrent treatment with an oral antidepressant selected from duloxetine, escitalopram, sertraline, or venlafaxine;
1.61. Any foregoing method, wherein the subject (e.g., patient) is unresponsive to, or cannot be treated with ketamine (e.g., S-ketamine), e.g., because it is contraindicated in said subject (e.g., patient).

In another aspect, the disclosure provides Compound of Formula I or a deuterated analog thereof, as hereinbefore described, in free or pharmaceutically acceptable salt form, for use in enhancing neural growth, enhancing neural connectivity, increasing synaptic density, increasing dendritic spine density, increasing dendritic spine size, and/or increasing excitatory neurotransmission, in the brain of a subject in need thereof, e.g., in the prefrontal cortex region of the brain, e.g., for use in any of Methods 1, et seq.

In another aspect, the disclosure provides the use of Compound of Formula I or a deuterated analog thereof, as hereinbefore described, in free or pharmaceutically acceptable salt form, in the manufacture of a medicament for enhancing neural growth, enhancing neural connectivity, increasing synaptic density, increasing dendritic spine density, increasing dendritic spine size, and/or increasing excitatory neurotransmission, in the brain of a subject in need thereof, e.g., in the medial prefrontal cortex region of the brain, e.g., for any of Methods 1, et seq.

In a second embodiment of the first aspect, the present disclosure provides a method (Method 2) for enhancing neural growth, enhancing neural connectivity, increasing synaptic density, increasing dendritic spine density, increasing dendritic spine size, and/or increasing excitatory neurotransmission, in the brain of a subject in need thereof, e.g., in the prefrontal cortex region of the brain, the method comprising the step of administering an effective amount of a Compound of Formula II (Compound II):

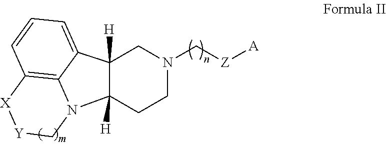

wherein:
X is selected from S, S(O), S(O)₂, O, CH₂, CHR^{b}, C(R^{b})₂, NH, N(R^{a}) (e.g., N(CH₃)), N—C(O)—R^{a}, N—C(O)—O—R^{a}, N—C(O)—O—CH₂—O—R^{a}, N—CH₂—O—C(O)—R^{a}, N⁺(═O⁻), a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Y is CH₂, CHR^{c}, —C(O)—, C(R^{c})₂, a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Z is a bond, —S—, S(O), S(O)₂, —O—, —NH, N(R^{d}), —C(O)—, —C(OH)—, —C(OC₁₋₆alkyl), —C(═N—OH)—, —C(═N—OC₁₋₆alkyl)-, a spiro-joined C₃₋₆cycloalkyl (e.g., cyclopropane), a spiro-joined 3-6-membered heterocycloalkyl (e.g., aziridine or oxetane), or —O(CH₂)ₚO— wherein p is 2, 3, or 4 (e.g., p is 2), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy; A is H, C₃₋₆cycloalkyl (e.g., cyclopropyl or cyclohexyl), aryl (e.g., phenyl), or heteroaryl, wherein said cycloalkyl, aryl, or heteroaryl is substituted by 0-5 groups R; each R is independently selected from aryl (e.g., phenyl), aryloxy (e.g., phenoxy), heteroaryl (e.g., pyridyl), C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy, ethoxy), C₁₋₆alkylthio (e.g., methylthio), halo (e.g., F), cyano, C₃₋₆cycloalkyl (e.g., cyclopropyl), C₃₋₆cycloalkoxy (e.g., cyclopropoxy), or hydroxy, wherein each of said aryl, heteroaryl, alkyl, haloalkyl, alkylsulfonyl, alkoxy, alkylthio, cycloalkyl, or cycloalkoxy, is optionally further substituted by one or more groups selected from aryl (optionally substituted with halo), halo, C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy), C₁₋₆alkylthio (e.g., methylthio), C₃₋₆cycloalkyl (e.g., cyclopropyl), C₃₋₆cycloalkoxy (e.g., cyclopropoxy), amino, C₁₋₆alkylamino (e.g., methylamino), di(C₁₋₆alkyl)amino (e.g., dimethylamino), (C₁₋₆alkyl) (C₁₋₆alkyl)amino (e.g., methylethylamino), and hydroxy; R^{a} and R^{d}, are each independently selected from C₁₋₂₀alkyl (e.g., methyl or tert-butyl), and C₁₋₂alkylaryl (e.g., benzyl or phenethyl);
R^{b} and R^{e} are each independently selected from C₁₋₆alkyl (e.g., methyl, ethyl, tert-butyl), C₁₋₆alkoxy, C₃₋₆cycloalkyl (e.g., cyclopropyl), C₃₋₆cycloalkoxy (e.g., cyclopropoxy), and C₁₋₂alkylaryl (e.g., benzyl or phenethyl);
m is 1 or 2; and
n is 1, 2, 3, 4, or 5;
in free or salt form (e.g., pharmaceutically acceptable salt form);
   provided that n is not 3 when Z is —C(O)—, X is CH₂ or O, and m is 2; and
   provided that n is not 3 when Z is —C(O)—, X is CH₂, and m is 1; and
   provided that n is not 3 when Z is —C(O)— or —O—, X is NH or N(R^{a}), and m is 1;
   and provided that n is not 3 when Z is O, X is NCH₃, Y is —C(O)—, and m is 1; to the subject.

In further embodiments of the second embodiment of the first aspect, the present disclosure provides:
2.1. Method 2, wherein in the Compound of Formula II X is S, S(O), or S(O)₂;
2.2. Method 2, wherein in the Compound of Formula II, X is O;
2.3. Method 2, wherein in the Compound of Formula II, X CH₂, CHR^{b}, or C(R^{b})₂;
2.4 Method 2.3, wherein R^{b} is independently C₁₋₆alkyl (e.g., methyl);
2.5 Method 2, wherein in the Compound of Formula II, X is CH₂;
2.6. Method 2, wherein in the Compound of Formula II, X is NH;
2.7 Method 2, wherein in the Compound of Formula II, X is N(R^{a});
2.8. Method 2, wherein in the Compound of Formula II, X is N—C(O)—R^{a};
2.9. Method 2, wherein in the Compound of Formula II, X is N—C(O)—O—R^{a};
2.10. Method 2, wherein in the Compound of Formula II, X is N—C(O)—O—CH₂—O—R^{a};
2.11. Method 2, wherein in the Compound of Formula II, X is N—CH₂—O—C(O)—R^{a};
2.12. Method 2, or any of 2.4-2.11, wherein in the Compound of Formula II, R^{a} is C₁₋₂alkylaryl (e.g., benzyl or phenethyl);
2.13. Method 2, or any of 2.4-2.11, wherein in the Compound of Formula II, R^{a} is C₁₋₂₀alkyl (e.g., methyl or tert-butyl);
2.14. Method 2, or any of 2.4-2.11, wherein in the Compound of Formula II, R^{a} is C₁₀₋₂₀alkyl (e.g., decyl or dodecyl);
2.15. Method 2, or any of 2.4-2.11, wherein in the Compound of Formula II, R^{a} is C₁₋₁₅alkyl (e.g., hexyl or octyl);
2.16. Method 2, or any of 2.4-2.11, wherein in the Compound of Formula II, R^{a} is C₇₋₁₅alkyl (e.g., heptyl or nonyl);
2.17. Method 2, or any of 2.4-2.11, wherein in the Compound of Formula II, R^{a} is C₁₋₆alkyl (e.g., butyl or hexyl);
2.18. Method 2, or any of 2.4-2.11, wherein in the Compound of Formula II, R^{a} is C₁₋₄alkyl (e.g., n-butyl or tert-butyl);
2.19. Method 2, or any of 2.4-2.11, wherein in the Compound of Formula II, R^{a} is C₁₋₃alkyl (e.g., propyl or isopropyl);
2.20. Method 2, or any of 2.4-2.11, wherein in the Compound of Formula II, R^{a} is C₁₋₂alkyl (e.g., methyl or ethyl);
2.21. Method 2, or any of 2.4-2.11, wherein in the Compound of Formula II, X is N(CH₃);
2.22. Method 2, wherein in the Compound of Formula II, X is a spiro-joined C₃₋₆cycloalkyl (e.g., cyclopropane);
2.23. Method 2.22, wherein in the Compound of Formula II, the spiro-joined C₃₋₆cycloalkyl is selected from cyclopropane, cyclobutane, cyclopentane, and cyclohexane;
2.24. Method 2.22, wherein in the Compound of Formula II, the spiro-joined C₃₋₆cycloalkyl is cyclopropane;
2.25. Method 2, wherein in the Compound of Formula II, X is spiro-joined 3-6-membered heterocycloalkyl (e.g., aziridine or oxetane);
2.26. Method 2.25, wherein in the Compound of Formula II, the spiro-joined 3-6-membered heterocycloalkyl is selected from aziridinc, azetidinc, oxctanc, pyrrolidine, tetrahydrofuran, piperidine, tetrahydropyran, piperazine, and morpholine;
2.27. Method 2.25, wherein in the Compound of Formula II, the spiro-joined 3-6-membered heterocycloalkyl is selected from aziridine;
2.28. Method 2, or any of 2.22-2.27, wherein said spiro-joined C₃₋₆cycloalkyl or 3-6-membered heterocycloalkyl is unsubstituted;
2.29. Method 2, or any of 2.22-2.27, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
2.30. Method 2, or any of 2.1-2.29, wherein in the Compound of Formula II, Y is CH₂;
2.31. Method 2, or any of 2.1-2.29, wherein in the Compound of Formula II, Y is —C(O)—;
2.32. Method 2, or any of 2.1-2.29, wherein in the Compound of Formula II, Y is CHR^{c} or C(R^{c})₂;
2.33. Compound 2.32, wherein in the Compound of Formula II, each R^{c} is independently C₁₋₆alkyl;
2.34. Compound 2.32, wherein in the Compound of Formula II, each R^{c} is independently selected from methyl, ethyl and propyl;
2.35. Method 2, or any of 2.1-2.29, wherein in the Compound of Formula II, Y is a spiro-joined C₃₋₆cycloalkyl (e.g., cyclopropane);
2.36. Method 2.35, wherein in the Compound of Formula II, the spiro-joined C₃₋₆cycloalkyl is selected from cyclopropane, cyclobutane, cyclopentane, and cyclohexane;
2.37. Method 2.35, wherein in the Compound of Formula II, the spiro-joined C₃₋₆cycloalkyl is cyclopropane;
2.38. Method 2, or any of 2.1-2.29, wherein in the Compound of Formula II, Y is spiro-joined 3-6-membered heterocycloalkyl (e.g., aziridine or oxetane);
2.39. Method 2.38, wherein in the Compound of Formula II, the spiro-joined 3-6-membered heterocycloalkyl is selected from aziridine, azetidine, oxctanc, pyrrolidine, tetrahydrofuran, piperidine, tetrahydropyran, piperazine, and morpholine;
2.40. Method 2.39, wherein in the Compound of Formula II, the spiro-joined 3-6-membered heterocycloalkyl is aziridine;
2.41. Method 2, or any of 2.35-2.40, wherein said spiro-joined C₃₋₆cycloalkyl or 3-6-membered heterocycloalkyl is unsubstituted;
2.42. Method 2, or any of 2.35-2.40, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
2.43. Method 2, or any of 2.1-2.42, wherein in the Compound of Formula II, Z is a bond;
2.44. Method 2, or any of 2.1-2.42, wherein in the Compound of Formula II, Z is S, S(O), or S(O)₂;
2.45. Method 2, or any of 2.1-2.42, wherein in the Compound of Formula II, Z is O;
2.46. Method 2, or any of 2.1-2.42, wherein in the Compound of Formula II, Z is NH;
2.47. Method 2, or any of 2.1-2.42, wherein in the Compound of Formula II, Z is N(R^{a}), e.g., N(CH₃);
2.48. Method 2, or any of 2.1-2.42, wherein in the Compound of Formula II, Z is-C(O)—;
2.49. Method 2, or any of 2.1-2.42, wherein Z is —C(OH)—, —C(OC₁₋₆alkyl), —C(═N—OH)—, —C(═N—OC₁₋₆alkyl)-, optionally wherein said C₁₋₆alkyl is methyl;
2.50. Method 2, or any of 2.1-2.42, wherein in the Compound of Formula II, Z is a spiro-joined C₃₋₆cycloalkyl (e.g., cyclopropane);
2.51. Method 2.50, wherein in the Compound of Formula II, the spiro-joined C₃₋₆cycloalkyl is selected from cyclopropane, cyclobutane, cyclopentane, and cyclohexane;
2.52. Method 2.50, wherein in the Compound of Formula II, the spiro-joined C₃₋₆cycloalkyl is cyclopropane;
2.53. Method 2, or any of 2.1-2.42, wherein in the Compound of Formula II, Z is spiro-joined 3-6-membered heterocycloalkyl (e.g., aziridine or oxetane);
2.54. Method 2.53, wherein in the Compound of Formula II, the spiro-joined 3-6-membered heterocycloalkyl is selected from aziridine, azetidine, oxetane, pyrrolidine, tetrahydrofuran, piperidine, tetrahydropyran, piperazine, and morpholine;
2.55. Method 2.53, wherein in the Compound of Formula II, the spiro-joined 3-6-membered heterocycloalkyl is aziridine;
2.56. Method 2, or any of 2.49-2.55, wherein said spiro-joined C₃₋₆cycloalkyl or 3-6-membered heterocycloalkyl is unsubstituted;
2.57. Method 2, or any of 2.49-2.55, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
2.58. Method 2, or any of 2.1-2.57, wherein in the Compound of Formula II, A is a 6-10 membered aryl ring, e.g., selected from phenyl and naphthyl, substituted by 0-5 groups R;
2.59. Method 2, or any of 2.1-2.57, wherein in the Compound of Formula II, A is a 5-10 membered heteroaryl ring, substituted by 0-5 groups R;
2.60. Method 2.59, wherein in the Compound of Formula II, A is selected from furan, thiophene (e.g., thiophen-2-yl), pyrrole, oxazole, thiazole, imidazole, isoxazole, isothiazole, pyrazole, pyridine (e.g., pyrid-4-yl), 2-oxopyridine (e.g., 2-oxopyridin-1 (2H)-yl), pyrimidine, pyridazine, pyrazine, benzofuran (e.g., benzofuran-4-yl, or benzofuran-7-yl, or 2-methylbenzofuran-4-yl), dihydrobenzofuran (e.g., 2,3-dihydrobenzofuran-7-yl), benzothiophene, indole (e.g., indol-1-yl, indol-3-yl, or indol-5-yl), benzoxazole, benzothiazole, benzimidazole (e.g., benzo[d]imidazol-1-yl), benzisoxazole (e.g., benzo[d]isoxazol-3-yl, or benzo[d]isoxazol-4-yl), benzisothiazole (e.g., benzo[d]isothiazol-3-yl), benzotriazole (e.g., benzo[d][1,2,3-triazol-1-yl), indazole (e.g., indazol-1-yl, indazol-3-yl, or indazol-7-yl), quinolinc (e.g., quinolin-8-yl), isoquinoline (e.g., isoquinolin-7-yl), quinazoline (e.g., quinazolin-7-yl), and quinoxaline (e.g., quinoxalin-5-yl);
2.61. Method 2.60, wherein in the Compound of Formula II, A is substituted by 0 groups R;
2.62. Method 2.60, wherein in the Compound of Formula II, A is substituted by 1 group R;
2.63. Method 2.60, wherein in the Compound of Formula II, A is substituted by 2 groups R;
2.64. Method 2.58, wherein in the Compound of Formula II, A is a phenyl ring, substituted by 0-5 groups R;
2.65. Method 2.64, wherein in the Compound of Formula II, there is one group R;
2.66. Method 2.64, wherein in the Compound of Formula II, the group R is positioned at the para position of the phenyl ring;
2.67. Method 2.64, wherein in the Compound of Formula II, the group R is positioned at the meta position of the phenyl ring;
2.68. Method 2.64, wherein in the Compound of Formula II, the group R is positioned at the ortho position of the phenyl ring;
2.69. Method 2.64, wherein in the Compound of Formula II, there are two groups R;
2.70. Method 2.69, wherein in the Compound of Formula II, the groups R are positioned at the ortho and para positions of the phenyl ring;
2.71. Method 2.69, wherein in the Compound of Formula II, the groups R are positioned at the meta and para positions of the phenyl ring;
2.72. Method 2.69, wherein in the Compound of Formula II, the groups R are positioned at the ortho and meta positions on the same side of the phenyl ring;
2.73. Method 2.69, wherein in the Compound of Formula II, the groups R are positioned at the ortho and meta positions on opposite sides of the phenyl ring;
2.74. Method 2.69, wherein in the Compound of Formula II, the groups R are positioned at the two ortho positions of the phenyl ring;
2.75. Method 2.69, wherein in the Compound of Formula II, the groups R are positioned at the two meta positions of the phenyl ring;
2.76. Method 2.64, wherein in the Compound of Formula II, there are three groups R;
2.77. Method 2.76, wherein in the Compound of Formula II, the groups R are positioned at the two ortho positions and the para position of the phenyl ring;
2.78. Method 2.64, wherein in the Compound of Formula II, there are four groups R;
2.79. Method 2.64, wherein in the Compound of Formula II, there are five groups R;
2.80. Method 2, or any of 2.1-2.79, wherein in the Compound of Formula II, each group R is independently selected from methyl, ethyl, trifluoromethyl, methoxy, ethoxy, F, Cl, cyano, hydroxy, 2-methoxyethoxy, methylsulfonyl, methylthio, cyclopropoxy, cyclopropylmethoxy, methylamino, 4-fluorophenoxy, and (4-fluorobenzyl)oxy;
2.81. Method 2, or any of 2.1-2.80, wherein in the Compound of Formula II, A is selected from the group consisting of: phenyl, 2-cyanophenyl, 3-cyanophenyl, 4-cyanophenyl, 2-methylphenyl, 2-ethylphenyl, 3-ethylphenyl, 4-ethylphenyl, 2-chlorophenyl, 3-chlorophenyl, 4-fluorophenyl, 3-chloro-4-fluorophenyl, 2-cyano-4-fluorophenyl, 3-cyano-4-fluorophenyl, 2-methyl-4-fluorophenyl, 3-methyl-4-fluorophenyl, 2-methoxy-4-fluorophenyl, 2-methoxy-5-fluorophenyl, 2-fluoro-4-methylphenyl, 2-methoxyphenyl, 3-methoxyphenyl, 4-methoxyphenyl, 2-ethoxyphenyl, 3-ethoxyphenyl, 2-hydroxyphenyl, 2,5-dimethoxyphenyl, 2-trifluoromethyoxyphenyl, 3-trifluoromethylphenyl, 2-(methylsulfonyl)phenyl, 3-(methylthio)phenyl, 4-(methoxyethoxy)phenyl, 4-(4-fluorobenzyloxy)phenyl, 4-(4-fluorophenoxy)phenyl, 3-cyclopropoxyphenyl, 3-(cyclopropylmethoxy)phenyl, and 2-(methylamino)phenyl;
2.82. Method 2, or any of 2.1-2.80, wherein in the Compound of Formula II, A is selected from the group consisting of: pyrid-4-yl, thiophen-2-yl, indol-1-yl, indol-3-yl, 5-fluoroindol-3-yl, indazol-1-yl, indazol-3-yl, indazol-7-yl, benzofuran-4-yl, benzofuran-7-yl, 2,3-dihydrobenzofuran-7-yl, 2-methylbenzofuran-7-yl, benzo[d]isoxazol-3-yl, benzo[d]isoxazol-4-yl, benzo[d]isoxazol-7-yl, 6-fluorobenzo[d]isoxazol-3-yl, benzo[d]isothiazol-3-yl, benzo[d]imidazole-1-yl, benzo[d][1,2,3]triazol-1-yl, isoquinolin-7-yl, quinolin-8-yl, quinoxaline-5-yl, quinazolin-7-yl, and 2-oxopyridin-1 (2H)-yl;
2.83. Method 2, or any of 2.1-2.80, wherein in the Compound of Formula II, A is selected from the group consisting of: phenyl, 2-ethylphenyl, 4-fluorophenyl, 2-methoxyphenyl, 3-methoxyphenyl, benzofuran-7-yl, benzo[d]isoxazol-3-yl, and benzo[d]isothiazol-3-yl;
2.84. Method 2, or any of 2.1-2.83, wherein in the Compound of Formula II, m is 1;
2.85. Method 2, or any of 2.1-2.83, wherein in the Compound of Formula II, m is 2;
2.86. Method 2, or any of 2.1-2.85, wherein in the Compound of Formula II, n is 2;
2.87. Method 2, or any of 2.1-2.85, wherein in the Compound of Formula II, n is 3;
2.88. Method 2, or any of 2.1-2.85, wherein in the Compound of Formula II, n is 4;
2.89. Method 2, or any of 2.1-2.85, wherein in the Compound of Formula II, n is 5;
2.90. Method 2, or any of 2.1-2.89, wherein in the Compound of Formula II, X is S, O, CH₂, NH, N(CH₃), or spiro-joined cyclopropyl; Y is CH₂, C(O), or spiro-joined cyclopropyl, and Z is a bond, —O—, —C(O)—, —O(CH₂)₂O—, or —C(═NOCH₃)—;
2.91. Method 2, or any of 2.1-2.89, wherein in the Compound of Formula II, X is N(CH₃) or spiro-joined cyclopropyl; Y is CH₂, C(O), or spiro-joined cyclopropyl, and Z is a bond, —O—, or —C(O)—;
2.92. Method 2, or any of 2.1-2.91, wherein the Compound of Formula II is a compound of Formula Ia:

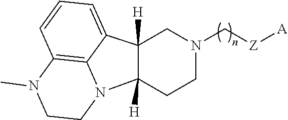

wherein n, Z, and A are defined as in any preceding embodiment;
2.93. Method 2, or any of 2.1-2.91, wherein the Compound of Formula II is a compound of Formula Ib:

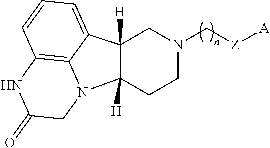

wherein n, Z, and A are defined as in any preceding embodiment;
2.94. Method 2, or any of 2.1-2.91, wherein the Compound of Formula II is a compound of Formula Ic:

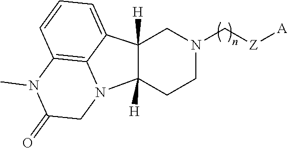

wherein n, Z, and A are defined as in any preceding embodiment;
2.95. Method 2, or any of 2.1-2.91, wherein the Compound of Formula II is a compound of Formula Id:

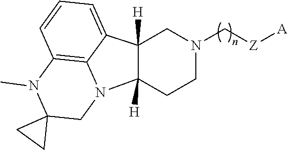

wherein n, Z, and A are defined as in any preceding embodiment;
2.96. Method 2, or any of 2.1-2.91, wherein the Compound of Formula II is a compound of Formula Ie:

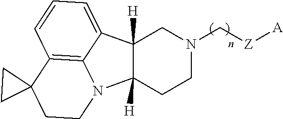

wherein n, Z, and A are defined as in any preceding embodiment;
2.97. Method 2, or any of 2.1-2.96, wherein in the Compound of Formula II, n is 4 and Z is a bond;
2.98. Method 2, or any of 2.1-2.96, wherein in the Compound of Formula II, n is 3 and Z is —O— or —C(O)—;
2.99. Method 2, or any of 2.1-2.96, wherein in the Compound of Formula II, n is 3 and Z is a bond;
2.100. Method 2, or any of 2.1-2.96, wherein in the Compound of Formula II, n is 2 and Z is —O— or —C(O)—;
2.101. Method 2, or any of 2.1-2.96, wherein in the Compound of Formula II, n is 2 and Z is a bond;
2.102. Method 2, or any of 2.1-2.96, wherein in the Compound of Formula II, n is 1 and Z is —O— or —C(O)—;
2.103. Method 2, or any of 2.1-2.96, wherein in the Compound of Formula II, n is 1 and Z is a bond;
2.104. Method 2, or any of 2.1-2.103, wherein in the Compound of Formula II, A is H or C₃₋₆cycloalkyl (e.g., cyclopropyl or cyclohexyl), Z is a bond or —C(O)—, and n is 1, 2, or 3;
2.105. Method 2, or any of 2.1-2.104, wherein the Compound of Formula II is selected from the group consisting of:

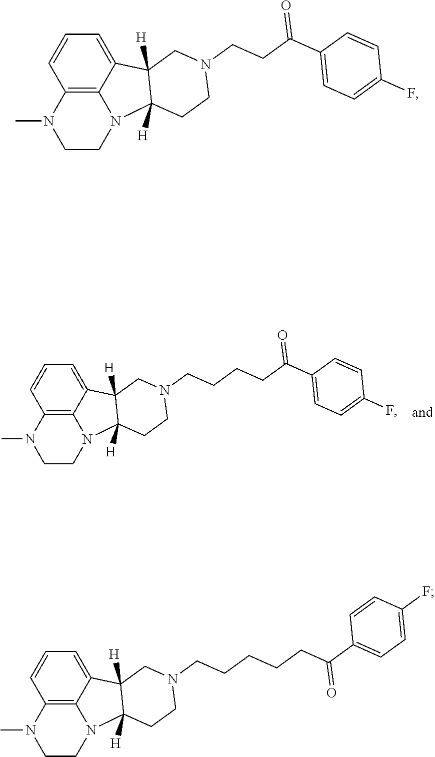 [SMILES: CN1CCN2c3c(cccc31)[C@@H]1CN(CCC(=O)c3ccc(F)cc3)CC[C@@H]12 ; CN1CCN2c3c(cccc31)[C@@H]1CN(CCCCC(=O)c3ccc(F)cc3)CC[C@@H]12 ; CN1CCN2c3c(cccc31)[C@@H]1CN(CCCCCC(=O)c3ccc(F)cc3)CC[C@@H]12]

each independently in free or pharmaceutically acceptable salt or form;
2.106. Method 2, or any of 2.1-2.105, wherein the Compound of Formula II is:

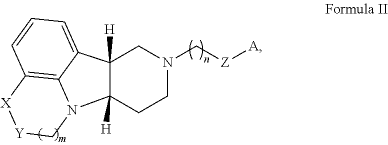

wherein the variables are defined as provided in any of the following embodiments:

| X | Y | m | n | Z | A |
|---|---|---|---|---|---|
| —N(CH₃)— | —CH₂— | 1 | 3 | —O— | 2-CN-4-F-phenyl |
| —N(CH₃)— | —CH₂— | 1 | 4 | —C(O)— | 4-F-phenyl |
| —N(CH₃)— | —CH₂— | 1 | 2 | —C(O)— | 4-F-phenyl |
| —N(CH₃)— | Cyp | 1 | 3 | —C(O)— | 4-F-phenyl |
| —N(CH₃)— | —CH₂— | 1 | 5 | —C(O)— | 4-F-phenyl |
| —N(CH₃)— | —CH₂— | 1 | 3 | —C(O)— | 4-(CH₃OCH₂CH₂O)- |
| | | | | | phenyl |
| Cyp | —CH₂— | 1 | 3 | —C(O)— | 4-F-phenyl |
| Cyp | —CH₂— | 1 | 3 | —O— | 4-F-phenyl |
| —NH— | —C(O)— | 1 | 3 | —O— | 3-Me-4-F-phenyl |
| —NH— | —C(O)— | 1 | 3 | —O— | 3-C1-4-F-phenyl |
| —NH— | —C(O)— | 1 | 3 | —O— | 3-CN-4-F-phenyl |
| —N(CH₃)— | —CH₂— | 1 | 2 | —O(CH₂)₂O— | 4-F-phenyl |
| —N(CH₃)— | —CH₂— | 1 | 3 | —C(O)— | 4-((4-F-benzyl)oxy) |
| —N(CH₃)— | Cyp | 1 | 2 | —C(O)— | 4-F-phenyl |
| —N(CH₃)— | Cyp | 1 | 2 | —O— | 4-F-phenyl |
| —N(CH₃)— | Cyp | 1 | 2 | —C(O)— | 2-Me-4-F-phenyl |
| —N(CH₃)— | Cyp | 1 | 2 | —O— | 2-Me-4-F-phenyl |
| —NH— | —C(O)— | 1 | 2 | —O— | 4-F-phenyl |
| —N(CH₃)— | Cyp | 1 | 3 | bond | 3-Cl-phenyl |
| —N(CH₃)— | Cyp | 1 | 3 | bond | 3-MeO-phenyl |
| —N(CH₃)— | Cyp | 1 | 3 | bond | 3-CF₃-phenyl |
| —N(CH₃)— | Cyp | 1 | 3 | bond | 2-Cl-phenyl |
| —N(CH₃)— | Cyp | 1 | 3 | bond | 2-Me-phenyl |
| —N(CH₃)— | Cyp | 1 | 2 | bond | 2-Cl-phenyl |
| —N(CH₃)— | Cyp | 1 | 2 | bond | 2-MeO-phenyl |
| —N(CH₃)— | —CH₂— | 1 | 2 | —O— | 4-F-phenyl |
| —N(CH₃)— | —CH₂— | 1 | 2 | —O— | 2-MeO-4-F-phenyl |
| —N(CH₃)— | —CH₂— | 1 | 3 | bond | 2-MeO-phenyl |
| —N(CH₃)— | —CH₂— | 1 | 3 | bond | 3-MeO-phenyl |
| —N(CH₃)— | —CH₂— | 1 | 3 | bond | 3-Cl-phenyl |
| —N(CH₃)— | —CH₂— | 1 | 3 | bond | 2-Cl-phenyl |
| —N(CH₃)— | —CH₂— | 1 | 3 | bond | 3-CF₃-phenyl |
| —N(CH₃)— | —CH₂— | 1 | 2 | bond | 2-Cl-phenyl |
| —N(CH₃)— | —CH₂— | 1 | 2 | bond | 2-MeO-phenyl |
| —N(CH₃)— | —CH₂— | 1 | 3 | bond | 2-Me-phenyl |
| —CH₂— | —CH₂— | 2 | 2 | —C(O)— | 4-F-phenyl |
| —CH₂— | —CH₂— | 2 | 2 | —O— | 4-F-phenyl |
| —CH₂— | —CH₂— | 2 | 2 | bond | 4-F-phenyl |
| —N(CH₃)— | Cyp | 1 | 3 | bond | 2-MeO-phenyl |
| —N(CH₃)— | Cyp | 1 | 2 | bond | benzo[d]isoxazol- |
| | | | | | 3-yl |
| —N(CH₃)— | —CH₂— | 1 | 2 | —C(O)— | 2-Me-4-F-phenyl |
| —O— | —CH₂— | 2 | 2 | —O— | 4-F-phenyl |
| —O— | —CH₂— | 2 | 2 | —C(O)— | 4-F-phenyl |
| —S— | —CH₂— | 2 | 2 | —C(O)— | 4-F-phenyl |
| —CH₂— | —CH₂— | 1 | 2 | —C(O)— | 4-F-phenyl |
| —S— | —CH₂— | 1 | 2 | —C(O)— | 4-F-phenyl |
| —NH— | Cyp | 1 | 2 | —O— | 4-F-phenyl |
| —NH— | Cyp | 1 | 2 | —O— | 2-Me-4-F-phenyl |
| —NH— | Cyp | 1 | 2 | —C(O)— | 4-F-phenyl |
| —NH— | Cyp | 1 | 2 | —C(O)— | 2-Me-4-F-phenyl |
| Cyp | —CH₂— | 1 | 2 | —O— | 4-F-phenyl |
| Cyp | —CH₂— | 1 | 2 | —O— | 2-Me-4-F-phenyl |
| Cyp | —CH₂— | 1 | 2 | —C(O)— | 4-F-phenyl |
| Cyp | —CH₂— | 1 | 2 | —C(O)— | 2-Me-4-F-phenyl |
| —NH— | —C(O)— | 1 | 2 | —C(O)— | 4-F-phenyl |
| —NH— | —C(O)— | 1 | 2 | —C(O)— | 2-Me-4-F-phenyl |
| —NH— | —C(O)— | 1 | 2 | —C(O)— | 2-F-4-Me-phenyl |
| —CH₂— | —CH₂— | 2 | 3 | bond | 2-MeO-phenyl |
| —CH₂— | —CH₂— | 2 | 3 | bond | 2-HO-phenyl |
| —CH₂— | —CH₂— | 2 | 3 | bond | 2-Cl-phenyl |
| —CH₂— | —CH₂— | 2 | 2 | bond | 2-Cl-phenyl |
| —CH₂— | —CH₂— | 2 | 3 | bond | 3-MeO-phenyl |
| —CH₂— | —CH₂— | 2 | 3 | bond | 3-CF₃-phenyl |
| —CH₂— | —CH₂— | 2 | 3 | bond | 3-Cl-phenyl |
| —N(CH₃)— | —CH₂— | 1 | 2 | bond | benzo[d]isoxazol- |
| | | | | | 3-yl |
| —N(CH₃)— | Cyp | 1 | 2 | bond | 6-F-benzo[d]isoxazol- |
| | | | | | 3-yl |
| —N(CH₃)— | —CH₂— | 1 | 2 | bond | 6-F-benzo[d]isoxazol- |
| | | | | | 3-yl |
| Cyp | —CH₂— | 1 | 3 | —O— | 2-Me-4-F-phenyl |
| —N(CH₃)— | —CH₂— | 1 | 3 | —C(O)— | 4-(4-F-PhO)-phenyl |
| Cyp | —CH₂— | 1 | 2 | bond | 2-MeO-phenyl |
| —N(CH₃)— | —CH₂— | 1 | 2 | bond | 3-MeO-phenyl |
| Cyp | —CH₂— | 1 | 2 | bond | benzo[d]isoxazol-3-yl |
| —N(CH₃)— | Cyp | 1 | 4 | bond | 2-CN-phenyl |
| —N(CH₃)— | Cyp | 1 | 2 | bond | 2-CN-phenyl |
| —N(CH₃)— | —CH₂— | 1 | 2 | bond | 2-CN-phenyl |
| —N(CH₃)— | —CH₂— | 1 | 3 | bond | 2-CN-phenyl |
| —N(CH₃)— | —CH₂— | 1 | 2 | bond | 2-oxopyridin-1(2H)-yl |
| —N(CH₃)— | —CH₂— | 1 | 2 | bond | 2-MeO-4-F-phenyl |
| —N(CH₃)— | Cyp | 1 | 2 | bond | 2-MeO-4-F-phenyl |
| —N(CH₃)— | —CH₂— | 1 | 2 | bond | 2-CF₃O-phenyl |
| —N(CH₃)— | —CH₂— | 1 | 2 | bond | 2-Et-phenyl |
| —N(CH₃)— | —CH₂— | 1 | 2 | bond | 4-MeO-phenyl |
| —N(CH₃)— | —CH₂— | 1 | 2 | bond | 3-CN-phenyl |
| —N(CH₃)— | —CH₂— | 1 | 2 | bond | 4-CN-phenyl |
| —N(CH₃)— | —CH₂— | 1 | 2 | bond | 5-F-indol-3-yl |
| —N(CH₃)— | —CH₂— | 1 | 3 | —C(O)— | 2-MeO-phenyl |
| —N(CH₃)— | —C(O)— | 1 | 2 | —O— | 4-F-phenyl |
| —N(CH₃)— | —C(O)— | 1 | 2 | bond | 2-MeO-phenyl |
| —N(CH₃)— | —CH₂— | 1 | 2 | bond | 2-HO-phenyl |
| —N(CH₃)— | —CH₂— | 1 | 1 | bond | 2-MeO-phenyl |
| —N(CH₃)— | —CH₂— | 1 | 2 | bond | 2-EtO-phenyl |
| —N(CH₃)— | —CH₂— | 1 | 2 | bond | benzofuran-7-yl |
| —N(CH₃)— | —CH₂— | 1 | 3 | 1—O— | 2-MeO-phenyl |
| —N(CH₃)— | —CH₂— | 1 | 2 | bond | benzo[d]isothiazol-3-yl |
| —N(CH₃)— | —CH₂— | 1 | 2 | bond | 2,5-di-MeO-phenyl |
| —N(CH₃)— | —CH₂— | 1 | 1 | bond | 4-Et-phenyl |
| —N(CH₃)— | —CH₂— | 1 | 2 | bond | indol-3-yl |
| —N(CH₃)— | Cyp | 1 | 2 | bond | 2-Et-phenyl |
| —N(CH₃)— | Cyp | 1 | 2 | bond | 3-MeO-phenyl |
| —N(CH₃)— | —CH₂— | 1 | 1 | bond | phenyl |
| —N(CH₃)— | —CH₂— | 1 | 1 | bond | 3-MeO-phenyl |
| —N(CH₃)— | —CH₂— | 1 | 1 | bond | 3-Et-phenyl |
| —N(CH₃)— | —CH₂— | 1 | 1 | bond | 4-MeO-phenyl |
| —N(CH₃)— | —CH₂— | 1 | 1 | bond | 2-Et-phenyl |
| —N⁺(═O)⁻— | Cyp | 1 | 2 | —O— | 4-F-phenyl |
| —N(CH₃)— | —CH₂— | 1 | 2 | bond | 2-MeSO₂-phenyl |
| —N(CH₃)— | Cyp | 1 | 2 | bond | benzo[d]isothiazol-3-yl |
| —N(CH₃)— | —CH₂— | 1 | 1 | bond | cyclohexyl |
| —N(CH₃)— | —CH₂— | 1 | 2 | bond | 2-MeO-5-F-phenyl |
| —N(CH₃)— | —CH₂— | 1 | 2 | bond | 3-Et-Phenyl |
| —N(CH₃)— | —CH₂— | 1 | 2 | bond | 3-EtO-phenyl |
| —N(CH₃)— | Cyp | 1 | 2 | bond | benzofuran-7-yl |
| —N(CH₃)— | —CH₂— | 1 | 2 | bond | benzo[d]imidazole-1-yl |
| —N(CH₃)— | —CH₂— | 1 | 2 | bond | benzo[d][1,2,3]triazol- |
| | | | | | 1-yl |
| —N(CH₃)— | —CH₂— | 1 | 2 | bond | indazol-3-yl |
| —NH— | —C(O)— | 1 | 2 | bond | benzo[d]isothiazol-3-yl |
| —N(CH₃)— | —C(O)— | 1 | 2 | bond | benzo[d]isothiazol-3-yl |
| —N(CH₃)— | Cyp | 1 | 1 | bond | 2-MeO-phenyl |
| —N(CH₃)— | —CH₂— | 1 | 1 | bond | benzo[d]isoxazol-3-yl |
| —N(CH₃)— | —CH₂— | 1 | 1 | bond | benzofuran-7-yl |
| —N(CH₃)— | —CH₂— | 1 | 1 | bond | benzo[d]isothiazol-3-yl |
| —N(CH₃)— | —CH₂— | 1 | 1 | bond | quinolin-8-yl |
| —N(CH₃)— | —CH₂— | 1 | 1 | bond | benzo[d]oxazol-7-yl |
| —N(CH₃)— | —CH₂— | 1 | 1 | bond | 2,3-dihydrobenzofuran- |
| | | | | | 7-yl |
| —N(CH₃)— | —CH₂— | 1 | 2 | —O— | 2-CN-4-F-phenyl |
| —N(CH₃)— | —CH₂— | 1 | 2 | bond | indazol-1-yl |
| —N(CH₃)— | —C(O)— | 1 | 2 | —C(O)— | 4-F-phenyl |
| —N(CH₃)— | —CH₂— | 1 | 1 | bond | quinoxalin-5-yl |
| —N(CH₃)— | —CH₂— | 1 | 1 | —C(O)— | 4-F-Phenyl |
| —N(CH₃)— | —CH₂— | 1 | 2 | —C(═NOMe) | 4-F-Ph |
| —N(CH₃)— | —CH₂— | 1 | 3 | —C(O)— | phenyl |
| —N(CH₃)— | —CH₂— | 1 | 2 | —C(═NOMe) | phenyl |
| —N(CH₃)— | —C(O)— | 1 | 2 | —C(O)— | phenyl |
| —N(CH₃)— | —CH₂— | 1 | 2 | —C(O)— | thiophen-2-yl |
| —N(CH₃)— | —CH₂— | 1 | 1 | bond | 3-(O-cyclo- |
| | | | | | propyl)phenyl |
| —N(CH₃)— | —CH₂— | 1 | 1 | bond | 3-(OCH₂- |
| | | | | | cyclopropyl)phenyl |
| —N(CH₃)— | —C(O)— | 1 | 2 | bond | 2-MeO-phenyl |
| —N(CH₃)— | —C(O)— | 1 | 2 | bond | 3-MeO-phenyl |
| —N(CH₃)— | —C(O)— | 1 | 2 | bond | benzo[d]isoxazol-3-yl |
| —N(CH₃)— | —CH₂— | 1 | 1 | bond | 3-MeS-phenyl |
| —N(CH₃)— | Cyp | 1 | 2 | —C(O)— | phenyl |
| —N(CH₃)— | Cyp | 1 | 3 | —C(OH)— | 4-F-phenyl |
| —N(CH₃)— | Cyp | 1 | 3 | —C(OH)— | phenyl |
| —N(CH₃)— | Cyp | 1 | 3 | —C(OMe)— | 4-F-phenyl |
| —N(CH₃)— | Cyp | 1 | 3 | —C(OMe)— | phenyl |
| —N(CH₃)— | —CH₂— | 1 | 2 | bond | 2-(MeNH)-phenyl |
| —N(CH₃)— | Cyp | 1 | 2 | bond | 2-HO-phenyl |
| —N(CH₃)— | Cyp | 1 | 2 | bond | 2-MeO-phenyl |
| —N(CH₃)— | Cyp | 1 | 2 | bond | 2-EtO-phenyl |
| —N(CH₃)— | Cyp | 1 | 2 | bond | 2-CF₃O-phenyl |
| —N(CH₃)— | Cyp | 1 | 2 | bond | 2-MeSO₂-phenyl |
| —N(CH₃)— | Cyp | 1 | 2 | bond | 2-(MeNH)-phenyl |
| —N(CH₃)— | Cyp | 1 | 2 | bond | 3-CN-phenyl |
| —N(CH₃)— | Cyp | 1 | 2 | bond | 4-MeO-phenyl |
| —N(CH₃)— | Cyp | 1 | 2 | bond | 4-CN-phenyl |
| —N(CH₃)— | —CH₂— | 1 | 1 | bond | quinazolin-7-yl |
| —N(CH₃)— | —CH₂— | 1 | 1 | bond | isoquinolin-7-yl |
| —N(CH₃)— | —CH₂— | 1 | 1 | bond | benzo[d]isoxazol-4-yl |
| —N(CH₃)— | —CH₂— | 1 | 1 | bond | benzofuran-4-yl |
| —N(CH₃)— | —CH₂— | 1 | 1 | bond | 2-Me-benzofuran-7-yl |
| —N(CH₃)— | —CH₂— | 1 | 2 | bond | 3-MeS-phenyl |
| —N(CH₃)— | —CH₂— | 1 | 2 | bond | 3-(O- |
| | | | | | Cyclopropyl)phenyl |
| —N(CH₃)— | —CH₂— | 1 | 2 | bond | 3-(OCH₂- |
| | | | | | Cyclopropyl)phenyl |
| —N(CH₃)— | —CH₂— | 1 | 2 | bond | benzo[d]isoxazol-4-yl |
| —N(CH₃)— | —CH₂— | 1 | 2 | bond | benzofuran-4-yl |
| —N(CH₃)— | —CH₂— | 1 | 2 | bond | 2-Me-benzofuran-7-yl |
| —N(CH₃)— | Cyp | 1 | 2 | bond | indazol-7-yl |
| —N(CH₃)— | Cyp | 1 | 2 | bond | quinolin-8-yl |
| —N(CH₃)— | Cyp | 1 | 2 | bond | pyrid-4-yl |
| —N(CH₃)— | —CH₂— | 1 | 2 | bond | quinolin-8-yl |
| —N(CH₃)— | —CH₂— | 1 | 2 | bond | indol-1-yl |
| —N(CH₃)— | —CH₂— | 1 | 2 | bond | pyrid-4-yl |
| —N(CH₃)— | Cyp | 1 | 1 | bond | 3-Et-phenyl |
| —N(CH₃)— | Cyp | 1 | 2 | —O— | 3-CN-4-F-phenyl |
| —N(CH₃)— | —CH₂— | 1 | 1 | bond | H |
| —N(CH₃)— | —CH₂— | 1 | 2 | bond | H |
| —N(CH₃)— | —CH₂— | 1 | 3 | bond | H |
| —N(CH₃)— | —CH₂— | 1 | 1 | bond | Cyclopentyl |
| —N(CH₃)— | —CH₂— | 1 | 1 | bond | Cyclobutyl |
| —N(CH₃)— | —CH₂— | 1 | 1 | bond | Cyclopropyl |

each independently in free or pharmaceutically acceptable salt or form, wherein Cyp refers to a spiro-joined cyclopropyl ring;
2.107. Method 2, or any of 2.1-2.105, wherein the Compound of Formula II is:

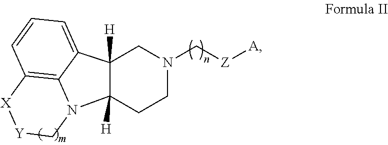

wherein the variables are defined as provided in any of the following embodiments:

| X | Y | m | n | Z | A |
|---|---|---|---|---|---|
| —N(CH₃)— | —CH₂— | 1 | 2 | —C(O)— | 4-F-phenyl |
| —N(CH₃)— | Cyp | 1 | 3 | —C(O)— | 4-F-phenyl |
| —N(CH₃)— | Cyp | 1 | 2 | —C(O)— | 4-F-phenyl |
| —N(CH₃)— | Cyp | 1 | 2 | —O— | 4-F-phenyl |
| —N(CH₃)— | Cyp | 1 | 2 | bond | 2-MeO-phenyl |
| —N(CH₃)— | Cyp | 1 | 2 | bond | benzo[d]isoxazol-3-yl |
| —N(CH₃)— | —CH₂— | 1 | 2 | bond | 3-MeO-phenyl |
| Cyp | —CH₂— | 1 | 2 | bond | benzo[d]isoxazol-3-yl |
| —N(CH₃)— | —CH₂— | 1 | 2 | bond | benzofuran-7-yl |
| —N(CH₃)— | —CH₂— | 1 | 2 | bond | benzo[d]isothiazol-3-yl |
| —N(CH₃)— | Cyp | 1 | 2 | bond | 2-Et-phenyl |
| —N(CH₃)— | Cyp | 1 | 2 | bond | 3-MeO-phenyl |
| —N(CH₃)— | Cyp | 1 | 2 | bond | benzo[d]isothiazol-3-yl |
| —N(CH₃)— | Cyp | 1 | 2 | bond | benzofuran-7-yl |
| —N(CH₃)— | —C(O)— | 1 | 2 | bond | benzo[d]isothiazol-3-yl |
| —N(CH₃)— | Cyp | 1 | 1 | bond | 2-MeO-phenyl |
| —N(CH₃)— | —C(O)— | 1 | 2 | —C(O)— | 4-F-phenyl |
| —N(CH₃)— | —C(O)— | 1 | 2 | bond | benzo[d]isoxazol-3-yl |
| —N(CH₃)— | Cyp | 1 | 2 | —C(O)— | Phenyl |
| —N(CH₃)— | Cyp | 1 | 1 | Bond | 3-Et-phenyl |

2.108. Method 2, or any of 2.1-2.107, wherein the Compound of Formula II is in free form;
2.109. Method 2, or any of 2.1-2.107, wherein the Compound of Formula II is in salt form, e.g., pharmaceutically acceptable salt form;
2.110. Method 2, or any of 2.1-2.107, wherein the Compound of Formula II is in acid addition salt form, for example, hydrochloric or toluenesulfonic acid salt form;
2.111. Method 2, or any of 2.1-2.110, wherein the Compound of Formula II is in substantially pure diastereomeric form (i.e., substantially free from other diastereomers);
2.112. Method 2 or any of 2.1-2.110, wherein the Compound of Formula II has a diastereomeric excess of greater than 70%, preferably greater than 80%, more preferably greater than 90% and most preferably greater than 95%;
2.113. Method 2 or any of 2.1-2.112, wherein the Compound of Formula II is in solid form, e.g., in crystal form;
2.114. Method 2 or any of 2.1-2.113, wherein the Compound of Formula II is in isolated or purified form (e.g., in at least 90% pure form, or at least 95% or at least 98% or at least 99%);
2.115. Method 2 or any of 2.1-2.114, wherein the Compound of Formula II has 5-HT_{2A} receptor binding affinity of at least 60% at 100 nM concentration, e.g., at least 70%, or at least 75%, or at least 80%, or at least 85%, or at least 90%, or at least 95%, or at least 98%, at 100 nM concentration;
2.116. Method 2 or any of 2.1-2.115, wherein the Compound of Formula II has a 5-HT_{2A} receptor dissociation constant (K_{d}) of less than 250 nM, or less than 100 nM, or less than 70 nM, or less than 60 nM, or less than 50 nM, or less than 40 nM, or less than 30 nM, or less than 20 nM, or less than 10 nM;
2.117. Method 2 or any of 2.1-2.116, wherein the Compound of Formula II is an agonist of beta-arrestin signaling via the 5-HT_{2A} receptor, e.g., a partial agonist or a full agonist;
2.118. Method 2.117, wherein the Compound of Formula II is a partial agonist of beta-arrestin signaling having an Eₘₐₓ of less than 90%, or less than 80%, or less than 70%, or less than 60%, or less than 50%, or less than 40%, or less than 30%, or less than 20%, or less than 10%, relative to a full agonist (e.g., alpha-methylserotonin);
2.119. Method 2.117 or 2.118, wherein the Compound of Formula II has an EC₅₀ for 5-HT_{2A} receptor beta-arrestin agonism of less than 500 nM, or less than 200 nM, or less than 150 nM, or less than 100 nM, or less than 70 nM, or less than 60 nM, or less than 50 nM, or less than 40 nM, or less than 30 nM, or less than 20 nM, or less than 10 nM;
2.120. Method 2.117, 2.118 or 2.119, wherein the Compound of Formula II has a beta-arrestin signaling relative intrinsic activity (RAᵢ) of less than 1.0 compared to the reference compound alpha-methylserotonin, e.g., a relative intrinsic activity of less than 0.8, or less than 0.6, or less than 0.5, or less than 0.4, or less than 0.3, or less than 0.2, or less than 0.1, or 0.1 to 0.8, or 0.2 to 0.8, or 0.4 to 0.8, or 0.5 to 0.8, or 0.2 to 0.6, or 0.2 to 0.5, or 0.2 to 0.4, or 0.5 to 1.0, or 0.5 to 0.9, or 0.5 to 0.8, or 0.6 to 0.9, or 0.6 to 0.8;
2.121. Method 2.117, 2.118, or 2.119, wherein the Compound of Formula II has a beta-arrestin signaling relative intrinsic activity (RAᵢ) of greater than 1.0 compared to the reference compound alpha-methylserotonin, e.g., a relative intrinsic activity of 1.0 to 1.2, or 1.0 to 1.4, or 1.0 to 1.6;
2.122. Method 2 or any of 2.1-2.116, wherein the Compound of Formula II is an antagonist of beta-arrestin signaling via the 5-HT_{2A} receptor;
2.123. Method 2.122, wherein the Compound of Formula II has an IC₅₀ for 5-HT_{2A} receptor beta-arrestin antagonism of less than 300 nM, or less than 200 nM, or less than 100 nM, or less than 70 nM, or less than 60 nM, or less than 50 nM, or less than 40 nM, or less than 30 nM, or less than 20 nM, or less than 10 nM;
2.124. Method 2 or any of 2.1-2.116, wherein the Compound of Formula II is not an antagonist of beta-arrestin signaling via the 5-HT_{2A} receptor;
2.125. Method 2.124, wherein the Compound of Formula II has an IC₅₀ for 5-HT_{2A} receptor beta-arrestin antagonism of greater than 10 nM, or greater than 50 nM, or greater than 100 nM, or greater than 250 nM, or greater than 500 nM, or greater than 1000 nM, or greater than 5000 nM, or greater than 10,000 nM;
2.126. Method 2 or any of 2.1-2.125, wherein the Compound of Formula II is not an agonist of G-q signaling via the 5-HT_{2A} receptor, or is a weak agonist thereof;
2.127. Method 2.126, wherein the Compound of Formula II is a partial agonist of G-q signaling having an Eₘₐₓ of less than 90%, or less than 80%, or less than 70%, or less than 60%, or less than 50%, or less than 40%, or less than 30%, or less than 20%, or less than 10%, relative to a full agonist (e.g., alpha-methylserotonin), preferably an Eₘₐₓ of less than 50% or less than 30% or less than 10%;
2.128. Method 2.126 or 2.127, wherein the Compound of Formula II has an EC₅₀ for 5-HT_{2A} receptor G-q agonism of greater than 10 nM, or greater than 25 nM, or greater than 50 nM, or greater than 100 nM, or greater than 150 nM, or greater than 200 nM, or greater than 500 nM, or greater than 1000 nM, or greater than 2000 nM, or greater than 5000 nM, or greater than 10,000 nM;
2.129. Method 2.126, 2.127 or 2.128, wherein the Compound of Formula II has a G-q signaling relative intrinsic activity (RAᵢ) of less than 1.0 compared to the reference compound alpha-methylserotonin, e.g., a relative intrinsic activity of less than 0.8, or less than 0.6, or less than 0.5, or less than 0.4, or less than 0.3, or less than 0.2, or less than 0.1, or 0.1 to 0.8, or 0.2 to 0.8, or 0.4 to 0.8, or 0.5 to 0.8, or 0.2 to 0.6, or 0.2 to 0.5, or 0.2 to 0.4, or 0.5 to 1.0, or 0.5 to 0.9, or 0.5 to 0.8, or 0.6 to 0.9, or 0.6 to 0.8;
2.130. Method 2 or any of 2.1-2.129, wherein the Compound of Formula II is an antagonist of G-q signaling via the 5-HT_{2A} receptor;
2.131. Method 2.130, wherein the Compound has an IC₅₀ for 5-HT_{2A} receptor G-q antagonism of less than 10 nM, or less than 25 nM, or less than 50 nM, or less than 100 nM, or less than 150 nM, or less than 200 nM, or less than 500 nM;
2.132. Method 2 or any of 2.1-2.131, wherein the Compound of Formula II has a bias ratio (beta-arrestin/G-q) for agonism 5-HT_{2A} receptor of at least 2, or at least 5, or at least 10, or at least 25, or at least 50, or at least 100, or at least 150, or at least 200 or at least 500, or at least 1000, or at least 10,000, or undefined (i.e., where the compound has any degree of beta-arrestin agonism and zero G-q agonism);
2.133. Method 2 or any of 2.1-2.132, wherein the Compound of Formula II is an antagonist or agonist of the D1 and/or D2 dopamine receptor (e.g., having at least 70% receptor affinity at 100 nM concentration or an IC₅₀ of less than 100 nM);
2.134. Method 2 or any of 2.1-2.132, wherein the Compound of Formula II is not active at the D1 and/or D2 dopamine receptor (e.g., having less than 50% receptor affinity at 100 nM concentration and/or an EC₅₀ or IC₅₀ of more than 500 nM);
2.135. Method 2 or any of 2.1-2.134, wherein the Compound of Formula II is an antagonist of the serotonin transporter (e.g., having at least 70% receptor binding affinity at 100 nM concentration or an IC₅₀ of less than 100 nM);
2.136. Method 2 or any of 2.1-2.134, wherein the Compound of Formula II is not active at the serotonin transporter (e.g., having less than 50% receptor binding affinity at 100 nM concentration and/or an EC₅₀ or IC₅₀ of more than 500 nM);
2.137. Method 2 or any of 2.1-2.136, wherein the Compound of Formula II is an agonist, antagonist, or partial agonist of the mu-opioid receptor (e.g., having at least 70% receptor binding affinity at 100 nM concentration or an EC₅₀ or IC₅₀ of less than 100 nM);
2.138. Method 2 or any of 2.1-2.136, wherein the Compound of Formula II is not active at the mu-opioid receptor (e.g., having at less than 50% receptor binding affinity at 100 nM concentration and/or an EC₅₀ or IC₅₀ of more than 500 nM);
2.139. Method 2, or any of 2.1-2.138, wherein the Compound of Formula II is non-hallucinogenic, e.g., at therapeutic doses for the treatment of a neuropsychiatric disorder described herein (e.g., depression, anxiety, etc.) the compound does not cause visual or auditory hallucinations, visual distortions (such as drifting, morphing, breathing or melting of objects and surfaces in the field of view), detachment from reality, dissociation, delirium, undesired altered states of consciousness;
2.140. Method 2, or any of 2.1-2.139, wherein the Compound of Formula II does not stimulate head twitch response in an animal test model, or is antagonist of DOI-induced head twitch response;
2.141. Method 2, or any of 2.1-2.140, wherein the Compound of Formula II is effective in a murine model of depression (tail suspension or forced swim test);
2.142. Method 2, or any of 2.1-2.141, wherein the Compound of Formula II is effective in an animal model of social anxiety disorder or anhedonia;
2.143. Method 2, or any of 2.1-2.142, wherein the Compound of Formula II does not have 5-HT_{2B} agonist activity (e.g., an EC50 of greater than 100 nM, or greater than 500 nM, or greater than 1000 nM, or greater than 10,000 nM);
2.144. Method 2, or any of 2.1-2.143, wherein the Compound of Formula II has 5-HT_{2B} antagonist activity (e.g., an IC₅₀ of less than 1000 nM, or less than 500 nM, or less than 250 nM, or less than 100 nM, or less than 50 nM, or less than 25 nM, or less than 15 nM);
2.145. Method 2, or any of 2.1-2.144, wherein the Compound of Formula II has 5-HT_{2C} agonist activity (e.g., an EC50 of less than 1000 nM, or less than 500 nM, or less than 250 nM, or less than 100 nM, or less than 50 nM, or less than 25 nM, or less than 15 nM);
2.146. Method 2, or any of 2.1-2.144, wherein the Compound of Formula II does not have 5-HT_{2C} antagonist activity (e.g., an IC₅₀ of greater than 100 nM, or greater than 500 nM, or greater than 1000 nM, or greater than 10,000 nM);
2.147. Method 2, or any of 2.1-2.146, wherein the Compound of Formula II binds to the alpha-1A adrenergic receptor (e.g., with a binding affinity Ki of less than 1000 nM, or less than 500 nM, or less than 250 nM, or less than 200 nM, or less than 150 nM, or less than 100 nM, or less than 50 nM, or less than 25 nM);
2.148. Method 2, or any of 2.1-2.147, wherein the Compound of Formula II does not cause psychoses (e.g., prolonged or intermittent psychoses);
2.149. Method 2, or any of 2.1-2.148, wherein the Compound of Formula II does not promote self-harm or harm to others in the patient;
2.150. Method 2, or any of 2.1-2.149, wherein the Compound of Formula II does not cause heart valvulopathy or pulmonary arterial hypertension, e.g., wherein the compound is safe to administer to a patient having cardiac comorbidities;
2.151. Method 2, or any of 2.1-2.150, wherein the Compound of Formula II does not cause abuse or dependence (e.g., physical or psychological dependence);
2.152. Method 2, or any of 2.1-2.151, wherein the Compound of Formula II is functionally inactive at one or more of the following receptors and ion channels: adenosine A2A, alpha-1A adrenergic, alpha-2A adrenergic, beta-1 adrenergic, beta-2 adrenergic, GABA-A benzodiazepine site (BZD, central), CB1 cannabinoid, CB2 cannabinoid, cholecystokinin CCK1, endothelin-A (ETA), NMDA, histamine H1, histamine H2, MAO-A, muscarinic M1, muscarinic M2, muscarinic M3, nicotinic acetylcholine (neuronal alpha-4-beta-2), delta opioid, kappa opioid, mu opioid, serotonin-1A, serotonin-1B, serotonin-3, glucocorticoid (GR), androgen (AR), vasopressin VIA, cardiac calcium channel (dihydropyridine site), hERG potassium channel, voltage-gated potassium channel Kv, sodium channel (site 2), norepinephrine transporter, dopamine transporter, and/or serotonin transporter;
2.153. Method 2.152, wherein the Compound of Formula II has an in vitro receptor activity (for agonism or antagonism) of less than 60% inhibition of radioligand binding (e.g., at 100 nM test concentration) for any one or more of said receptor or ion channels, e.g., less than 50%, less than 40%, less than 30%, less than 20%, or less than 10%;
2.154. Method 2, or any of 2.1-2.153, wherein the Compound of Formula II is orally bioavailable (e.g., oral bioavailability of at least 10%, or at least 15%, or at least 20%, or at least 30%, or at least 40%);
2.155. Any foregoing method, wherein the method provides enhanced neural growth in the brain of the subject (e.g., in the prefrontal cortex region of the brain);
2.156. Any foregoing method, wherein the method provides enhanced neural connectivity, in the brain of the subject (e.g., in the prefrontal cortex region of the brain);
2.157. Any foregoing method, wherein the method provides increased synaptic density, in the brain of the subject (e.g., in the prefrontal cortex region of the brain);
2.158. Any foregoing method, wherein the method provides increased dendritic spine density, in the brain of the subject (e.g., in the prefrontal cortex region of the brain);
2.159. Any foregoing method, wherein the method provides increased dendritic spine lengths), in the brain of the subject (e.g., in the prefrontal cortex region of the brain);
2.160. Any foregoing method, wherein the method provides increased excitatory neurotransmission (e.g., enhanced glutamatergic transmission or increased rate of mEPSCs), in the brain of the subject (e.g., in the prefrontal cortex region of the brain);
2.161. Method 2, or any of 2.1-2.160, wherein the method provides the enhanced neural growth, enhanced neural connectivity, increased synaptic density, increased dendritic spine density, increased dendritic spine size, and/or increased excitatory neurotransmission, in the brain of the subject (e.g., in the medial prefrontal cortex region of the brain) within less than 4 weeks of the initiation of administration of the Compound of Formula II, e.g., less than 3 weeks, less than 2 weeks, less than 1 week, less than 6 days, less than 5 days, less than 4 days, less than 3 days, or less than 2 days, after the initiation of treatment with the Compound of Formula II;
2.162. Method 2, or any of 2.1-2.161, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the Compound of Formula II, e.g., for at least 3 weeks, or at least 4 weeks, or at least 2 months, or at least 3 months;
2.163. Method 2, or any of 2.1-2.162, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the Compound of Formula II, e.g., for at least 3 weeks, or at least 4 weeks, or at least 2 months, or at least 3 months, such as by measured by in vivo imaging (e.g., MRI);
2.164. Method 2, or any of 2.1-2.163, wherein the enhanced neural growth, enhanced neural connectivity, increased synaptic density, increased dendritic spine density, increased dendritic spine size, and/or increased excitatory neurotransmission is characterized by or caused by enhanced neuritogenesis and/or enhanced neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
2.165. Method 2, or any of 2.1-2.164, wherein the method enhances neuritogenesis and/or enhances neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
2.166. Method 2.123 or 2.165, wherein the enhanced neuritogenesis or enhanced neurite outgrowth is characterized by increases in one or more of: the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization (e.g., measured or estimated using a Sholl Analysis);
2.167. Any foregoing method, wherein the method does not cause hallucinogenic side effects;
2.168. Any foregoing method, wherein the Compound of Formula II is administered in a daily dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base;
2.169. Any foregoing method, wherein the Compound of Formula II is administered in a dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base, at a frequency of every other day, or every two days, or every three days, or every four days, or every five days, or every six days, or every seven days;
2.170. Method 2, or any of 2.1-2.169, wherein the Compound of Formula II is administered as a unit dosage form for oral administration (e.g., enteral), for example, a tablet or capsule;
2.171. Method 2.170, wherein the unit dosage for oral administration (e.g., enteral), for example a tablet or capsule, comprises the Compound of Formula II in an amount equivalent 1 to 100 mg of free base, e.g., 1 to 75 mg, or 1 to 60 mg, or 1 to 40 mg, or 1 to 20 mg, or 1 to 10 mg, of free base, and a pharmaceutically acceptable diluent or carrier;
2.172. Method 2, or any of 2.1-2.171, wherein the Compound of Formula II is administered as a unit dosage for subcutaneous or transmucosal administration, e.g., a sublingual or buccal orally disintegrating tablet or film;
2.173. Method 2.172, wherein the unit dosage for subcutaneous or transmucosal administration, e.g., a sublingual or buccal orally disintegrating tablet or film, comprises the Compound of Formula II in an amount equivalent to 0.5 to 30 mg of free base, e.g., 1-10 mg of free base, and a pharmaceutically acceptable diluent or carrier;
2.174. Method 2, or any of 2.1-2.173, wherein the Compound of Formula II is administered as a long-acting injectable (LAI) composition, e.g., for intramuscular or subcutaneous injection;
2.175. Method 2.174, wherein the dose of the LAI composition is sufficient to provide the equivalent of a daily dose of 1 to 100 mg of free base of the Compound of Formula II, e.g., 1 to 75 mg, or 1 to 60 mg, or 1 to 40 mg, or 1 to 20 mg, or 1 to 10 mg, of free base, released over a period of time ranging from about 1 week to about 3 months, e.g., about 1 week to about 8 weeks, or about 1 week to about 6 weeks, or about 1 week to about 4 weeks, or about 1 week to about 3 weeks, or about 1 week to about 2 weeks;
2.176. Method 2.174 or 2.175, wherein the LAI composition comprises the Compound of Formula II dissolved, dispersed, suspended, or encapsulated in a polymeric matrix;
2.177. Method 2.176, wherein the polymeric matrix comprises one or more biocompatible and biodegradable polymers as defined herein, e.g., poly(hydroxycarboxylic acids), poly(amino acids), cellulose polymers, modified cellulose polymers, polyamides, and polyesters;
2.178. Method 2.177, wherein the one or more polymers comprises polylactic acid, polyglycolic acid, polycitric acid, polymalic acid, poly-beta-hydroxybutyric acid, poly(lactic acid-glycolic acid) copolymer, 2-hydroxybutyric acid-glycolic acid copolymer, polylactic acid-polyethylene glycol copolymer, polyglycolic acid-polyethylene glycol copolymer, poly(alkyl alpha-cyanoacrylate) such as poly(butyl cyanoacrylate) or poly(2-octyl cyanoacrylate), poly(ortho ester), polycarbonate, polyortho-carbonate, a polyamino acid, (for example poly-gamma.-L-alanine, poly-.gamma.-benzyl-L-glutamic acid or poly-y-methyl-L-glutamic acid), and/or hyaluronic acid ester;
2.179. Method 2.177, wherein the one or more polymers comprises polylactic acid, polyglycolic acid, polycitric acid, polymalic acid, or a poly(lactic acid-glycolic acid) copolymer;
2.180. Method 2.177, wherein the one or more polymers comprises a poly(lactic acid-glycolic acid) copolymer, e.g., poly-d,l-lactide-co-glycolide;
2.181. Any foregoing method, wherein the subject is an animal;
2.182. Any foregoing method, wherein the subject is a human (e.g., a patient suffering from a neuropsychiatric disorder);
2.183. Method 2.182, wherein the subject is a patient suffering from anxiety or depression, e.g., bipolar depression, major depressive disorder (MDD), post-traumatic stress disorder, or treatment-resistant depression;
2.184. Method 2.183, wherein the subject is a patient suffering from treatment resistant depression (e.g., depression which has not responded to treatment with an antidepressant agent selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof);
2.185. Method 2.184, wherein the subject is a patient suffering from bipolar depression or major depressive disorder;
2.186. Any foregoing method, wherein the method further comprises the concurrent administration of an anti-depressant agent (e.g., selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof), e.g., administered simultaneously, separately or sequentially;
2.187. Any foregoing method, wherein the method further comprises the concurrent administration of an NMDA receptor antagonist, for example, selected from ketamine (e.g., S-ketamine and/or R-ketamine), hydroxynorketamine, memantine, dextromethorphan, dextroallorphan, dextrorphan, amantadine, and agmatine, or any combination thereof, e.g., administered simultaneously, separately or sequentially;
2.188. Any foregoing method, wherein the method further comprises the concurrent administration of a NMDA receptor allosteric modulator, e.g., a NMDA receptor glycine-site modulator, such as rapastinel, nebostinel, apimostinel, D-cycloserine, or any combination thereof, e.g., administered simultaneously, separately or sequentially;
2.189. Any foregoing method, wherein the subject has previously been treated with but has not responded to, or has not responded adequately to, or who suffers undesirable side effects from, treatment with another antidepressant agent, for example, any one or more of a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor, or a serotonin receptor antagonist;
2.190. Any foregoing method, wherein the Compound of Formula II is administered as monotherapy, e.g., it is not administered concurrently or in conjunction with an anti-depressant, anti-psychotic, or anti-anxiety agent;
2.191. Any foregoing method, wherein the Compound of Formula II is administered without the direct supervision of a health care professional (e.g., the compound is self-administered by the subject (e.g., patient));
2.192. Any foregoing method, wherein the method does not comprise supervision or observation of the subject (e.g., patient) by a health care professional during or after (e.g., within 2 hours after) administration of a dose of the Compound of Formula II;
2.193. Any foregoing method, wherein the method does not put the subject (e.g., patient) at risk for sedation, dissociation, abuse, misuse, or suicidal ideation;
2.194. Any foregoing method, wherein the method does not result in hypertension (e.g., systolic and/or diastolic hypertension) within four hours after administration of a dose of the Compound of Formula II, e.g., an increase of more than 10 mm Hg, or more than 20 mm Hg, or more than 30 mm Hg, or more than 40 mm Hg, in systolic and/or diastolic blood pressure within 30 minutes to 4 hours after said dose;
2.195. Any foregoing method, wherein the method does not result in cognitive decline in the subject (e.g., patient);
2.196. Any foregoing method, wherein the subject (e.g., patient) has been diagnosed with or is at risk of aneurysmal vascular disease (e.g., thoracic aorta, abdominal aorta, intracranial, or peripheral arterial aneurysms), arteriovenous malformation or intracerebral hemorrhage;
2.197. Any foregoing method, wherein the subject (e.g., patient) is under concurrent treatment with an oral antidepressant selected from duloxetine, escitalopram, sertraline, or venlafaxine;
2.198. Any foregoing method, wherein the subject (e.g., patient) is not under concurrent treatment with an oral antidepressant selected from duloxetine, escitalopram, sertraline, or venlafaxine;
2.199. Any foregoing method, wherein the subject (e.g., patient) is unresponsive to, or cannot be treated with ketamine (e.g., S-ketamine), e.g., because it is contraindicated in said subject (e.g., patient).

In another aspect, the disclosure provides Compound of Formula II, as hereinbefore described, in free or pharmaceutically acceptable salt form, for use in enhancing neural growth, enhancing neural connectivity, increasing synaptic density, increasing dendritic spine density, increasing dendritic spine size, and/or increasing excitatory neurotransmission, in the brain of a subject in need thereof, e.g., in the prefrontal cortex region of the brain, e.g., for use in any of Methods 2, et seq.

In another aspect, the disclosure provides the use of Compound of Formula II, as hereinbefore described, in free or pharmaceutically acceptable salt form, in the manufacture of a medicament for enhancing neural growth, enhancing neural connectivity, increasing synaptic density, increasing dendritic spine density, increasing dendritic spine size, and/or increasing excitatory neurotransmission, in the brain of a subject in need thereof, e.g., in the medial prefrontal cortex region of the brain, e.g., for any of Methods 2, et seq.

In a first embodiment of the second aspect, the present disclosure provides a method (Method 3) for enhancing neuritogenesis and/or neurite outgrowth in the brain of a subject in need thereof, e.g., in the prefrontal cortex region of the brain, the method comprising the step of administering an effective amount of a Compound of Formula I (Compound I):

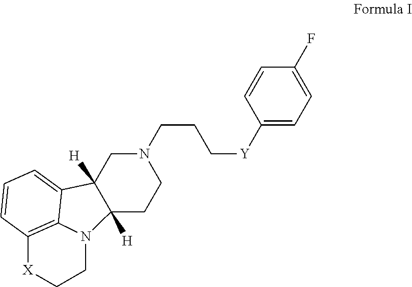

wherein X is selected from —O—, —S—, —N(H), and —N(CH₃)—, and Y is selected from —O—, —C(O)—, —CH(OH)—, and —CH(OCH₃);

or a deuterated analog thereof, in free, or pharmaceutically acceptable salt form, to the subject.

In further embodiments of the second aspect, the present disclosure provides:
3.1. Method 3, wherein in the Compound of Formula I, X is —O— or —S—;
3.2. Method 3, wherein in the Compound of Formula I, X is —N(H) or —N(CH₃)—;
3.3. Method 3, wherein in the Compound of Formula I, X is —N(CH₃)—;
3.4. Method 3, or any of 3.1-3.3, wherein in the Compound of Formula I, Y is —O—;
3.5 Method 3, or any of 3.1-3.3, wherein in the Compound of Formula I, Y is —C(O)—;
3.6. Method 3, or any of 3.1-3.3, wherein in the Compound of Formula I, Y is —CH(OH)—;
3.7. Method 3, or any of 3.1-3.6, wherein the Compound of Formula I is selected from the group consisting of:

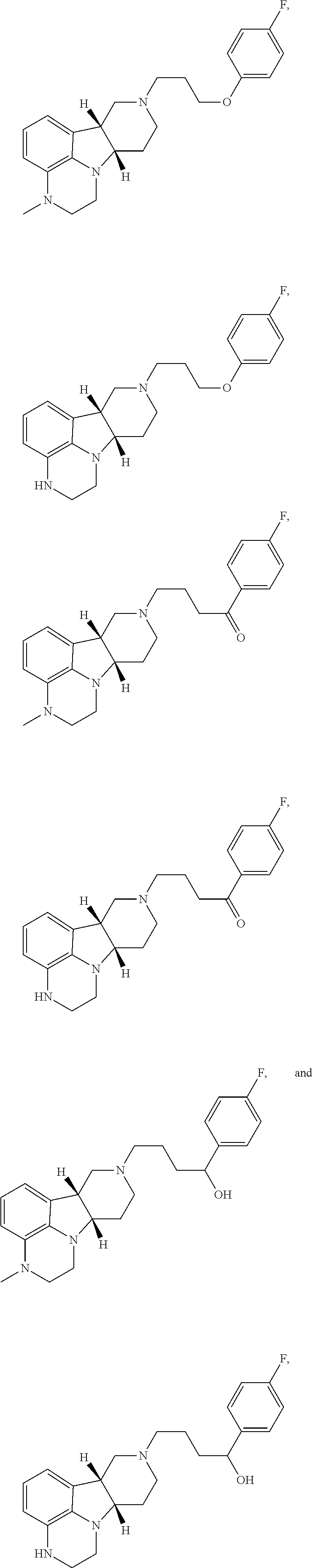

or a deuterated analog thereof;
3.8. Method 3, or any of 3.1-3.7, wherein the Compound of Formula I is selected from:

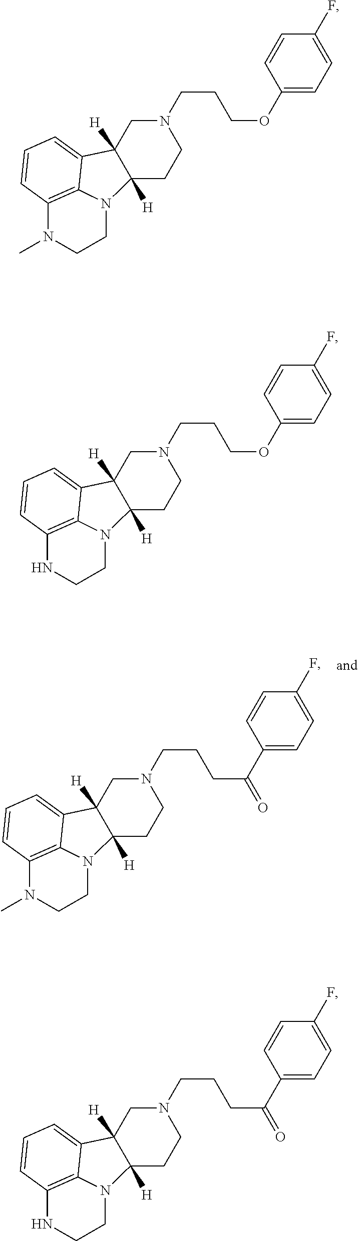 [SMILES: CN1CCN2c3c(cccc31)[C@@H]1CN(CCCOc3ccc(F)cc3)CC[C@@H]12 ; Fc1ccc(OCCCN2CC[C@H]3[C@@H](C2)c2cccc4c2N3CCN4)cc1 ; CN1CCN2c3c(cccc31)[C@@H]1CN(CCCC(=O)c3ccc(F)cc3)CC[C@@H]12 ; O=C(CCCN1CC[C@H]2[C@@H](C1)c1cccc3c1N2CCN3)c1ccc(F)cc1]

or a deuterated analog thereof;
3.9. Method 3, or any of 3.1-3.7, wherein the Compound of Formula I is selected from:

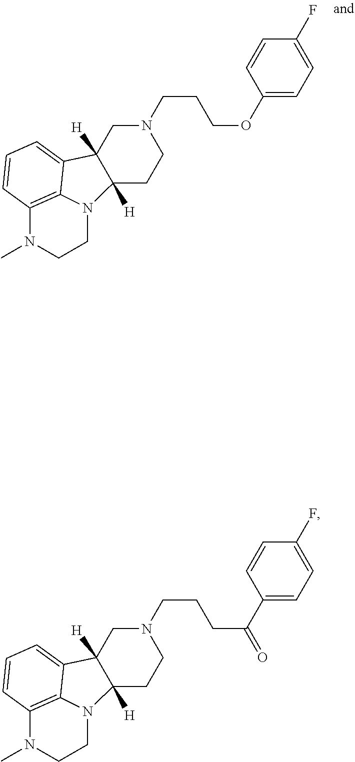 [SMILES: CN1CCN2c3c(cccc31)[C@@H]1CN(CCCOc3ccc(F)cc3)CC[C@@H]12 ; CN1CCN2c3c(cccc31)[C@@H]1CN(CCCC(=O)c3ccc(F)cc3)CC[C@@H]12]

or a deuterated analog thereof;
3.10. Method 3, or any of 3.1-3.7, wherein the Compound of Formula I is:

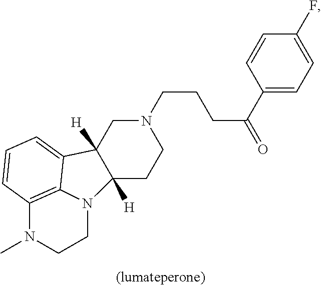 [SMILES: CN1CCN2c3c(cccc31)[C@@H]1CN(CCCC(=O)c3ccc(F)cc3)CC[C@@H]12]

or a deuterated analog thereof;
3.11. Method 3, or any of 3.1-3.10, wherein the Compound of Formula I or deuterated analog thereof is in the form of a free base;
3.12. Method 3, or any of 3.1-3.11, wherein the Compound of Formula I or deuterated analog thereof is in the form of a pharmaceutically acceptable salt;
3.13. Method 3.12, wherein the pharmaceutically acceptable salt is a toluenesulfonic acid addition salt (e.g., a mono-tosylate salt or a bis-tosylate salt);
3.14. Method 3 or any of 3.1-3.13, wherein the Compound of Formula I or deuterated analog thereof is non-deuterated lumateperone, i.e., having the following structure:

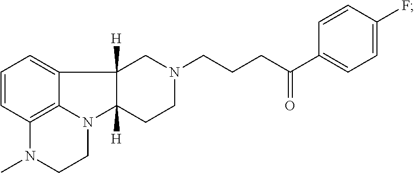 [SMILES: CN1CCN2c3c(cccc31)[C@@H]1CN(CCCC(=O)c3ccc(F)cc3)CC[C@@H]12]

3.15. Method 3 or any of 3.1-3.14, wherein the Compound of Formula I or salt thereof is in deuterated form, e.g., wherein the deuterium: protium ratio for at least one specified carbon-bound hydrogen atom is significantly higher, e.g., at least 2×, for example at least 10× higher, than the natural isotope ratios;
3.16. Method 3.15 wherein the deuterated analog of the Compound of Formula I is selected from

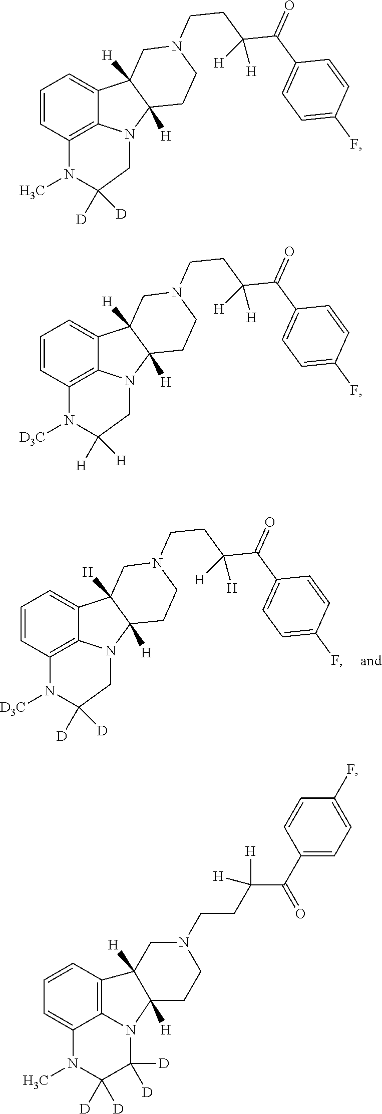 [SMILES: [2H]C1([2H])CN2c3c(cccc3N1C)[C@@H]1CN(CCCC(=O)c3ccc(F)cc3)CC[C@@H]12 ; [2H]C([2H])([2H])N1CCN2c3c(cccc31)[C@@H]1CN(CCCC(=O)c3ccc(F)cc3)CC[C@@H]12 ; [2H]C([2H])([2H])N1c2cccc3c2N(CC1([2H])[2H])[C@H]1CCN(CCCC(=O)c2ccc(F)cc2)C[C@@H]31 ; "and" ; [2H]C1([2H])N(C)c2cccc3c2N([C@H]2CCN(CCCC(=O)c4ccc(F)cc4)C[C@@H]32)C1([2H])[2H]]

in free or pharmaceutically acceptable salt form;
wherein D represents a hydrogen position with substantially greater than natural deuterium incorporation (i.e., substantially greater than 0.0156%), e.g., greater than 60%, or greater than 70%, or greater than 80%, or greater than 90% or greater than 95%, or greater than 96%, or greater than 97%, or greater than 98%, or greater than 99%, in free or pharmaceutically acceptable salt form, e.g., tosylate salt form;
3.17. Any foregoing method, wherein the method provides enhanced neural growth in the brain of the subject (e.g., in the prefrontal cortex region of the brain);
3.18. Any foregoing method, wherein the method provides enhanced neural connectivity, in the brain of the subject (e.g., in the prefrontal cortex region of the brain);
3.19. Any foregoing method, wherein the method provides increased synaptic density, in the brain of the subject (e.g., in the prefrontal cortex region of the brain);
3.20. Any foregoing method, wherein the method provides increased dendritic spine density, in the brain of the subject (e.g., in the prefrontal cortex region of the brain);
3.21. Any foregoing method, wherein the method provides increased dendritic spine size (e.g., increased width of spine heads and/or increased spine protrusion lengths), in the brain of the subject (e.g., in the prefrontal cortex region of the brain);
3.22. Any foregoing method, wherein the method provides increased excitatory neurotransmission (e.g., enhanced glutamatergic transmission or increased rate of mEPSCs), in the brain of the subject (e.g., in the prefrontal cortex region of the brain);
3.23. Method 3, or any of 3.1-3.22, wherein the method provides the enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the medial prefrontal cortex region of the brain) within less than 4 weeks of the initiation of administration of the Compound of Formula I or deuterated analog thereof, e.g., less than 3 weeks, less than 2 weeks, less than 1 week, less than 6 days, less than 5 days, less than 4 days, less than 3 days, or less than 2 days, after the initiation of treatment with the Compound of Formula I or deuterated analog thereof;
3.24. Method 3, or any of 3.1-3.23, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the Compound of Formula I or deuterated analog thereof, e.g., for at least 3 weeks, or at least 4 weeks, or at least 2 months, or at least 3 months;
3.25. Method 3, or any of 3.1-3.24, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the Compound of Formula I or deuterated analog thereof, e.g., for at least 3 weeks, or at least 4 weeks, or at least 2 months, or at least 3 months, such as by measured by in vivo imaging (e.g., MRI);
3.26. Method 3, or any of 3.1-3.25, wherein the enhanced neural growth, enhanced neural connectivity, increased synaptic density, increased dendritic spine density, increased dendritic spine size, and/or increased excitatory neurotransmission is caused by the enhanced neuritogenesis and/or enhanced neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
3.27. Method 3, or any of 3.1-3.26, wherein the method enhances neuritogenesis and/or enhances neurite outgrowth within less than 4 weeks of the initiation of administration of the Compound of Formula I or deuterated analog thereof, e.g., less than 3 weeks, less than 2 weeks, less than 1 week, less than 6 days, less than 5 days, less than 4 days, less than 3 days, or less than 2 days, after the initiation of treatment with the Compound of Formula I or deuterated analog thereof;
3.28. Method 3, or any of 3.1-3.27, wherein the enhanced neuritogenesis or enhanced neurite outgrowth is characterized by increases in one or more of: the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization (e.g., measured or estimated using a Sholl Analysis);
3.29. Any foregoing method, wherein the method does not cause hallucinogenic side effects;
3.30. Any foregoing method, wherein the Compound of Formula I or deuterated analog thereof is administered in a daily dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base;
3.31. Any foregoing method, wherein the Compound of Formula I or deuterated analog thereof is administered in a dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base, at a frequency of every other day, or every two days, or every three days, or every four days, or every five days, or every six days, or every seven days;
3.32. Method 3, or any of 3.1-3.31, wherein the Compound of Formula I or deuterated analog thereof is administered as a unit dosage form for oral administration (e.g., enteral), for example, a tablet or capsule;
3.33. Method 3.32, wherein the unit dosage for oral administration (e.g., enteral), for example a tablet or capsule, comprises the Compound of Formula I or deuterated analog thereof in an amount equivalent 1 to 100 mg of free base, e.g., 1 to 75 mg, or 1 to 60 mg, or 1 to 40 mg, or 1 to 20 mg, or 1 to 10 mg, of free base, and a pharmaceutically acceptable diluent or carrier;
3.34. Method 3, or any of 3.1-3.31, wherein the Compound of Formula I or deuterated analog thereof is administered as a unit dosage for subcutaneous or transmucosal administration, e.g., a sublingual or buccal orally disintegrating tablet or film;
3.35. Method 3.34, wherein the unit dosage for subcutaneous or transmucosal administration, e.g., a sublingual or buccal orally disintegrating tablet or film, comprises the Compound of Formula I or deuterated analog thereof in an amount equivalent to 0.5 to 30 mg of free base, e.g., 1-10 mg of free base, and a pharmaceutically acceptable diluent or carrier;
3.36. Method 3, or any of 3.1-3.31, wherein the Compound of Formula I or deuterated analog thereof is administered as a long-acting injectable (LAI) composition, e.g., for intramuscular or subcutaneous injection;
3.37. Method 3.36, wherein the dose of the LAI composition is sufficient to provide the equivalent of a daily dose of 1 to 100 mg of free base of the Compound of Formula I or deuterated analog thereof, e.g., 1 to 75 mg, or 1 to 60 mg, or 1 to 40 mg, or 1 to 20 mg, or 1 to 10 mg, of free base, released over a period of time ranging from about 1 week to about 3 months, e.g., about 1 week to about 8 weeks, or about 1 week to about 6 weeks, or about 1 week to about 4 weeks, or about 1 week to about 3 weeks, or about 1 week to about 2 weeks;
3.38. Method 3.36 or 3.37, wherein the LAI composition comprises the Compound of Formula I or deuterated analog thereof dissolved, dispersed, suspended, or encapsulated in a polymeric matrix;
3.39. Method 3.38, wherein the polymeric matrix comprises one or more biocompatible and biodegradable polymers as defined herein, e.g., poly(hydroxycarboxylic acids), poly(amino acids), cellulose polymers, modified cellulose polymers, polyamides, and polyesters;
3.40. Method 3.39, wherein the one or more polymers comprises polylactic acid, polyglycolic acid, polycitric acid, polymalic acid, poly-beta-hydroxybutyric acid, poly(lactic acid-glycolic acid) copolymer, 2-hydroxybutyric acid-glycolic acid copolymer, polylactic acid-polyethylene glycol copolymer, polyglycolic acid-polyethylene glycol copolymer, poly(alkyl alpha-cyanoacrylate) such as poly(butyl cyanoacrylate) or poly(2-octyl cyanoacrylate), poly(ortho ester), polycarbonate, polyortho-carbonate, a polyamino acid, (for example poly-gamma.-L-alanine, poly-.gamma.-benzyl-L-glutamic acid or poly-y-methyl-L-glutamic acid), and/or hyaluronic acid ester;
3.41. Method 3.39, wherein the one or more polymers comprises polylactic acid, polyglycolic acid, polycitric acid, polymalic acid, or a poly(lactic acid-glycolic acid) copolymer;
3.42. Method 3.39, wherein the one or more polymers comprises a poly(lactic acid-glycolic acid) copolymer, e.g., poly-d,l-lactide-co-glycolide;
3.43. Any foregoing method, wherein the subject is an animal;
3.44. Any foregoing method, wherein the subject is a human (e.g., a patient suffering from a neuropsychiatric disorder);
3.45. Method 3.44, wherein the subject is a patient suffering from anxiety or depression, e.g., bipolar depression, major depressive disorder (MDD), post-traumatic stress disorder, or treatment-resistant depression;
3.46. Method 3.45, wherein the subject is a patient suffering from treatment resistant depression (e.g., depression which has not responded to treatment with an antidepressant agent selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof);
3.47. Method 3.45, wherein the subject is a patient suffering from bipolar depression or major depressive disorder;
3.48. Any foregoing method, wherein the method further comprises the concurrent administration of an anti-depressant agent (e.g., selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof), e.g., administered simultaneously, separately or sequentially;
3.49. Any foregoing method, wherein the method further comprises the concurrent administration of an NMDA receptor antagonist, for example, selected from ketamine (e.g., S-ketamine and/or R-ketamine), hydroxynorketamine, memantine, dextromethorphan, dextroallorphan, dextrorphan, amantadine, and agmatine, or any combination thereof, e.g., administered simultaneously, separately or sequentially;
3.50. Any foregoing method, wherein the method further comprises the concurrent administration of a NMDA receptor allosteric modulator, e.g., a NMDA receptor glycine-site modulator, such as rapastinel, nebostinel, apimostinel, D-cycloserine, or any combination thereof, e.g., administered simultaneously, separately or sequentially;
3.51. Any foregoing method, wherein the subject has previously been treated with but has not responded to, or has not responded adequately to, or who suffers undesirable side effects from, treatment with another antidepressant agent, for example, any one or more of a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor, or a serotonin receptor antagonist;
3.52. Any foregoing method, wherein the Compound of Formula I or deuterated analog thereof is administered as monotherapy, e.g., it is not administered concurrently or in conjunction with an anti-depressant, anti-psychotic, or anti-anxiety agent;
3.53. Any foregoing method, wherein the Compound of Formula I or deuterated analog thereof is administered without the direct supervision of a health care professional (e.g., the compound is self-administered by the subject (e.g., patient));
3.54. Any foregoing method, wherein the method does not comprise supervision or observation of the subject (e.g., patient) by a health care professional during or after (e.g., within 2 hours after) administration of a dose of the Compound of Formula I or deuterated analog thereof;
3.55. Any foregoing method, wherein the method does not put the subject (e.g., patient) at risk for sedation, dissociation, abuse, misuse, or suicidal ideation;
3.56. Any foregoing method, wherein the method does not result in hypertension (e.g., systolic and/or diastolic hypertension) within four hours after administration of a dose of the Compound of Formula I or deuterated analog thereof, e.g., an increase of more than 10 mm Hg, or more than 20 mm Hg, or more than 30 mm Hg, or more than 40 mm Hg, in systolic and/or diastolic blood pressure within 30 minutes to 4 hours after said dose;
3.57. Any foregoing method, wherein the method does not result in cognitive decline in the subject (e.g., patient);
3.58. Any foregoing method, wherein the subject (e.g., patient) has been diagnosed with or is at risk of aneurysmal vascular disease (e.g., thoracic aorta, abdominal aorta, intracranial, or peripheral arterial aneurysms), arteriovenous malformation or intracerebral hemorrhage;
3.59. Any foregoing method, wherein the subject (e.g., patient) is under concurrent treatment with an oral antidepressant selected from duloxetine, escitalopram, sertraline, or venlafaxine;
3.60. Any foregoing method, wherein the subject (e.g., patient) is not under concurrent treatment with an oral antidepressant selected from duloxetine, escitalopram, sertraline, or venlafaxine;
3.61. Any foregoing method, wherein the subject (e.g., patient) is unresponsive to, or cannot be treated with ketamine (e.g., S-ketamine), e.g., because it is contraindicated in said subject (e.g., patient);
3.62. Any foregoing method, wherein the enhanced neuritogenesis and/or enhanced neurite outgrowth is associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density.

In a second embodiment of the second aspect, the present disclosure provides a method (Method 4) for enhancing neuritogenesis and/or neurite outgrowth in the brain of a subject in need thereof, e.g., in the prefrontal cortex region of the brain, the method comprising the step of administering an effective amount of a Compound of Formula II (Compound II):

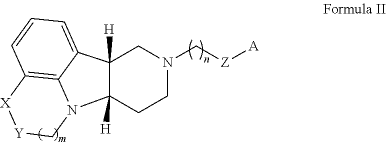

wherein:
X is selected from S, S(O), S(O)₂, O, CH₂, CHR^{b}, C(R^{b})₂, NH, N(R^{a}) (e.g., N(CH₃)), N—C(O)—R^{a}, N—C(O)—O—R^{a}, N—C(O)—O—CH₂—O—R^{a}, N—CH₂—O—C(O)—R^{a}, N⁺(═O⁻), a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Y is CH₂, CHR^{c}, —C(O)—, C(R^{c})₂, a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Z is a bond, —S—, S(O), S(O)₂, —O—, —NH, N(R^{d}), —C(O)—, —C(OH)—, —C(OC₁₋₆alkyl), —C(═N—OH)—, —C(═N—OC₁₋₆alkyl)-, a spiro-joined C₃₋₆cycloalkyl (e.g., cyclopropane), a spiro-joined 3-6-membered heterocycloalkyl (e.g., aziridine or oxetane), or —O(CH₂)ₚO— wherein p is 2, 3, or 4 (e.g., p is 2), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy; A is H, C₃₋₆cycloalkyl (e.g., cyclopropyl or cyclohexyl), aryl (e.g., phenyl), or heteroaryl, wherein said cycloalkyl, aryl, or heteroaryl is substituted by 0-5 groups R;
each R is independently selected from aryl (e.g., phenyl), aryloxy (e.g., phenoxy), heteroaryl (e.g., pyridyl), C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy, ethoxy), C₁₋₆alkylthio (e.g., methylthio), halo (e.g., F), cyano, C₃₋₆cycloalkyl (e.g., cyclopropyl), C₃₋₆cycloalkoxy (e.g., cyclopropoxy), or hydroxy, wherein each of said aryl, heteroaryl, alkyl, haloalkyl, alkylsulfonyl, alkoxy, alkylthio, cycloalkyl, or cycloalkoxy, is optionally further substituted by one or more groups selected from aryl (optionally substituted with halo), halo, C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy), C₁₋₆alkylthio (e.g., methylthio), C₃₋₆cycloalkyl (e.g., cyclopropyl), C₃₋₆cycloalkoxy (e.g., cyclopropoxy), amino, C₁₋₆alkylamino (e.g., methylamino), di(C₁₋₆alkyl)amino (e.g., dimethylamino), (C₁₋₆alkyl) (C₁₋₆alkyl)amino (e.g., methylethylamino), and hydroxy;
R^{a} and R^{d}, are each independently selected from C₁₋₂₀alkyl (e.g., methyl or tert-butyl), and C₁₋₂alkylaryl (e.g., benzyl or phenethyl);
R^{b} and R^{e} are each independently selected from C₁₋₆alkyl (e.g., methyl, ethyl, tert-butyl), C₁₋₆alkoxy, C₃₋₆cycloalkyl (e.g., cyclopropyl), C₃₋₆cycloalkoxy (e.g., cyclopropoxy), and C₁₋₂alkylaryl (e.g., benzyl or phenethyl);
m is 1 or 2;
n is 1, 2, 3, 4, or 5;
in free or salt form (e.g., pharmaceutically acceptable salt form);
   provided that n is not 3 when Z is —C(O)—, X is CH₂ or O, and m is 2; and
   provided that n is not 3 when Z is —C(O)—, X is CH₂, and m is 1; and
   provided that n is not 3 when Z is —C(O)— or —O—, X is NH or N(R^{a}), and m is 1;
   and provided that n is not 3 when Z is O, X is NCH₃, Y is —C(O)—, and mis 1; to the subject.

In further embodiments of the second embodiment of the second aspect, the present disclosure provides:
4.1. Method 4, wherein in the Compound of Formula II X is S, S(O), or S(O)₂;
4.2. Method 4, wherein in the Compound of Formula II, X is O;
4.3. Method 4, wherein in the Compound of Formula II, X CH₂, CHR^{b}, or C(R^{b})₂;
4.4 Method 4.3, wherein R^{D} is independently C₁₋₆alkyl (e.g., methyl);
4.5 Method 4, wherein in the Compound of Formula II, X is CH₂;
4.6. Method 4, wherein in the Compound of Formula II, X is NH;
4.7. Method 4, wherein in the Compound of Formula II, X is N(R^{a});
4.8. Method 4, wherein in the Compound of Formula II, X is N—C(O)—R^{a};
4.9. Method 4, wherein in the Compound of Formula II, X is N—C(O)—O—R^{a};
4.10. Method 4, wherein in the Compound of Formula II, X is N—C(O)—O—CH₂—O—R^{a};
4.11. Method 4, wherein in the Compound of Formula II, X is N—CH₂—O—C(O)—R^{a};
4.12. Method 4, or any of 4.4-4.11, wherein in the Compound of Formula II, R^{a} is C₁₋₂alkylaryl (e.g., benzyl or phenethyl);
4.13. Method 4, or any of 4.4-4.11, wherein in the Compound of Formula II, R^{a} is C₁₋₂₀alkyl (e.g., methyl or tert-butyl);
4.14. Method 4, or any of 4.4-4.11, wherein in the Compound of Formula II, R^{a} is C₁₀₋₂₀alkyl (e.g., decyl or dodecyl);
4.15. Method 4, or any of 4.4-4.11, wherein in the Compound of Formula II, R^{a} is C₁₋₁₅alkyl (e.g., hexyl or octyl);
4.16. Method 4, or any of 4.4-4.11, wherein in the Compound of Formula II, R^{a} is C₇₋₁₅alkyl (e.g., heptyl or nonyl);
4.17. Method 4, or any of 4.4-4.11, wherein in the Compound of Formula II, R^{a} is C₁₋₆alkyl (e.g., butyl or hexyl);
4.18. Method 4, or any of 4.4-4.11, wherein in the Compound of Formula II, R^{a} is C₁₋₄alkyl (e.g., n-butyl or tert-butyl);
4.19. Method 4, or any of 4.4-4.11, wherein in the Compound of Formula II, R^{a} is C₁₋₃alkyl (e.g., propyl or isopropyl);
4.20. Method 4, or any of 4.4-4.11, wherein in the Compound of Formula II, R^{a} is C₁₋₂alkyl (e.g., methyl or ethyl);
4.21. Method 4, or any of 4.4-4.11, wherein in the Compound of Formula II, X is N(CH₃);
4.22. Method 4, wherein in the Compound of Formula II, X is a spiro-joined C₃₋₆cycloalkyl (e.g., cyclopropane);
4.23. Method 4.22, wherein in the Compound of Formula II, the spiro-joined C₃₋₆cycloalkyl is selected from cyclopropane, cyclobutane, cyclopentane, and cyclohexane;
4.24. Method 4.22, wherein in the Compound of Formula II, the spiro-joined C₃₋₆cycloalkyl is cyclopropane;
4.25. Method 4, wherein in the Compound of Formula II, X is spiro-joined 3-6-membered heterocycloalkyl (e.g., aziridine or oxctanc);
4.26. Method 4.25, wherein in the Compound of Formula II, the spiro-joined 3-6-membered heterocycloalkyl is selected from aziridine, azetidine, oxetane, pyrrolidine, tetrahydrofuran, piperidine, tetrahydropyran, piperazine, and morpholine;
4.27. Method 4.25, wherein in the Compound of Formula II, the spiro-joined 3-6-membered heterocycloalkyl is selected from aziridine;
4.28. Method 4, or any of 4.22-4.27, wherein said spiro-joined C₃₋₆cycloalkyl or 3-6-membered heterocycloalkyl is unsubstituted;
4.29. Method 4, or any of 4.22-4.27, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
4.30. Method 4, or any of 4.1-4.29, wherein in the Compound of Formula II, Y is CH₂;
4.31. Method 4, or any of 4.1-4.29, wherein in the Compound of Formula II, Y is —C(O)—;
4.32. Method 4, or any of 4.1-4.29, wherein in the Compound of Formula II, Y is CHR^{c} or C(R^{c})₂;
4.33. Compound 4.32, wherein in the Compound of Formula II, each Reis independently C₁₋₆alkyl;
4.34. Compound 4.32, wherein in the Compound of Formula II, each Reis independently selected from methyl, ethyl and propyl;
4.35. Method 4, or any of 4.1-4.29, wherein in the Compound of Formula II, Y is a spiro-joined C₃₋₆cycloalkyl (e.g., cyclopropane);
4.36. Method 4.35, wherein in the Compound of Formula II, the spiro-joined C₃₋₆cycloalkyl is selected from cyclopropane, cyclobutane, cyclopentane, and cyclohexane;
4.37. Method 4.35, wherein in the Compound of Formula II, the spiro-joined C₃₋₆cycloalkyl is cyclopropane;
4.38. Method 4, or any of 4.1-4.29, wherein in the Compound of Formula II, Y is spiro-joined 3-6-membered heterocycloalkyl (e.g., aziridine or oxctanc);
4.39. Method 4.38, wherein in the Compound of Formula II, the spiro-joined 3-6-membered heterocycloalkyl is selected from aziridine, azetidine, oxetane, pyrrolidine, tetrahydrofuran, piperidine, tetrahydropyran, piperazine, and morpholine;
4.40. Method 4.39, wherein in the Compound of Formula II, the spiro-joined 3-6-membered heterocycloalkyl is aziridine;
4.41. Method 4, or any of 4.35-4.40, wherein said spiro-joined C₃₋₆cycloalkyl or 3-6-membered heterocycloalkyl is unsubstituted;
4.42. Method 4, or any of 4.35-4.40, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
4.43. Method 4, or any of 4.1-4.42, wherein in the Compound of Formula II, Z is a bond;
4.44. Method 4, or any of 4.1-4.42, wherein in the Compound of Formula II, Z is S, S(O), or S(O)₂;
4.45. Method 4, or any of 4.1-4.42, wherein in the Compound of Formula II, Z is O;
4.46. Method 4, or any of 4.1-4.42, wherein in the Compound of Formula II, Z is NH;
4.47. Method 4, or any of 4.1-4.42, wherein in the Compound of Formula II, Z is N(R^{a}), e.g., N(CH₃);
4.48. Method 4, or any of 4.1-4.42, wherein in the Compound of Formula II, Z is-C(O)—;
4.49. Method 4, or any of 4.1-4.42, wherein in the Compound of Formula II, Z is-C(OH)—, —C(OC₁₋₆alkyl), —C(═N—OH)—, —C(═N—OC₁₋₆alkyl)-, optionally wherein said C₁₋₆alkyl is methyl;
4.50. Method 4, or any of 4.1-4.42, wherein in the Compound of Formula II, Z is a spiro-joined C₃₋₆cycloalkyl (e.g., cyclopropane);
4.51. Method 4.50, wherein in the Compound of Formula II, the spiro-joined C₃₋₆cycloalkyl is selected from cyclopropane, cyclobutane, cyclopentane, and cyclohexane;
4.52. Method 4.50, wherein in the Compound of Formula II, the spiro-joined C₃₋₆cycloalkyl is cyclopropane;
4.53. Method 4, or any of 4.1-4.42, wherein in the Compound of Formula II, Z is spiro-joined 3-6-membered heterocycloalkyl (e.g., aziridine or oxetane);
4.54. Method 4.53, wherein in the Compound of Formula II, the spiro-joined 3-6-membered heterocycloalkyl is selected from aziridine, azetidine, oxetane, pyrrolidine, tetrahydrofuran, piperidine, tetrahydropyran, piperazine, and morpholine;
4.55. Method 4.53, wherein in the Compound of Formula II, the spiro-joined 3-6-membered heterocycloalkyl is aziridine;
4.56. Method 4, or any of 4.49-4.55, wherein said spiro-joined C₃₋₆cycloalkyl or 3-6-membered heterocycloalkyl is unsubstituted;
4.57. Method 4, or any of 4.49-4.55, wherein said spiro-joined C₃₋₆cycloalkyl or 3-6-membered heterocycloalkyl is substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
4.58. Method 4, or any of 4.1-4.57, wherein in the Compound of Formula II, A is a 6-10 membered aryl ring, e.g., selected from phenyl and naphthyl, substituted by 0-5 groups R;
4.59. Method 4, or any of 4.1-4.57, wherein in the Compound of Formula II, A is a 5-10 membered heteroaryl ring, substituted by 0-5 groups R;
4.60. Method 4.59, wherein in the Compound of Formula II, A is selected from furan, thiophene (e.g., thiophen-2-yl), pyrrole, oxazole, thiazole, imidazole, isoxazole, isothiazole, pyrazole, pyridine (e.g., pyrid-4-yl), 2-oxopyridine (e.g., 2-oxopyridin-1 (2H)-yl), pyrimidine, pyridazine, pyrazine, benzofuran (e.g., benzofuran-4-yl, or benzofuran-7-yl, or 2-methylbenzofuran-4-yl), dihydrobenzofuran (e.g., 2,3-dihydrobenzofuran-7-yl), benzothiophene, indole (e.g., indol-1-yl, indol-3-yl, or indol-5-yl), benzoxazole, benzothiazole, benzimidazole (e.g., benzo[d]imidazol-1-yl), benzisoxazole (e.g., benzo[d]isoxazol-3-yl, or benzo[d]isoxazol-4-yl), benzisothiazole (e.g., benzo[d]isothiazol-3-yl), benzotriazole (e.g., benzo[d][1,2,3-triazol-1-yl), indazole (e.g., indazol-1-yl, indazol-3-yl, or indazol-7-yl), quinoline (e.g., quinolin-8-yl), isoquinoline (e.g., isoquinolin-7-yl), quinazoline (e.g., quinazolin-7-yl), and quinoxaline (e.g., quinoxalin-5-yl);
4.61. Method 4.60, wherein in the Compound of Formula II, A is substituted by 0 groups R;
4.62. Method 4.60, wherein in the Compound of Formula II, A is substituted by 1 group R;
4.63. Method 4.60, wherein in the Compound of Formula II, A is substituted by 2 groups R;
4.64. Method 4.58, wherein in the Compound of Formula II, A is a phenyl ring, substituted by 0-5 groups R;
4.65. Method 4.64, wherein in the Compound of Formula II, there is one group R;
4.66. Method 4.64, wherein in the Compound of Formula II, the group R is positioned at the para position of the phenyl ring;
4.67. Method 4.64, wherein in the Compound of Formula II, the group R is positioned at the meta position of the phenyl ring;
4.68. Method 4.64, wherein in the Compound of Formula II, the group R is positioned at the ortho position of the phenyl ring;
4.69. Method 4.64, wherein in the Compound of Formula II, there are two groups R;
4.70. Method 4.69, wherein in the Compound of Formula II, the groups R are positioned at the ortho and para positions of the phenyl ring;
4.71. Method 4.69, wherein in the Compound of Formula II, the groups R are positioned at the meta and para positions of the phenyl ring;
4.72. Method 4.69, wherein in the Compound of Formula II, the groups R are positioned at the ortho and meta positions on the same side of the phenyl ring;
4.73. Method 4.69, wherein in the Compound of Formula II, the groups R are positioned at the ortho and meta positions on opposite sides of the phenyl ring;
4.74. Method 4.69, wherein in the Compound of Formula II, the groups R are positioned at the two ortho positions of the phenyl ring;
4.75. Method 4.69, wherein in the Compound of Formula II, the groups R are positioned at the two meta positions of the phenyl ring;
4.76. Method 4.64, wherein in the Compound of Formula II, there are three groups R;
4.77. Method 4.76, wherein in the Compound of Formula II, the groups R are positioned at the two ortho positions and the para position of the phenyl ring;
4.78. Method 4.64, wherein in the Compound of Formula II, there are four groups R;
4.79. Method 4.64, wherein in the Compound of Formula II, there are five groups R;
4.80. Method 4, or any of 4.1-4.79, wherein in the Compound of Formula II, each group R is independently selected from methyl, ethyl, trifluoromethyl, methoxy, ethoxy, F, Cl, cyano, hydroxy, 2-methoxyethoxy, methylsulfonyl, methylthio, cyclopropoxy, cyclopropylmethoxy, methylamino, 4-fluorophenoxy, and (4-fluorobenzyl)oxy;
4.81. Method 4, or any of 4.1-4.80, wherein in the Compound of Formula II, A is selected from the group consisting of: phenyl, 2-cyanophenyl, 3-cyanophenyl, 4-cyanophenyl, 2-methylphenyl, 2-ethylphenyl, 3-ethylphenyl, 4-ethylphenyl, 2-chlorophenyl, 3-chlorophenyl, 4-fluorophenyl, 3-chloro-4-fluorophenyl, 2-cyano-4-fluorophenyl, 3-cyano-4-fluorophenyl, 2-methyl-4-fluorophenyl, 3-methyl-4-fluorophenyl, 2-methoxy-4-fluorophenyl, 2-methoxy-5-fluorophenyl, 2-fluoro-4-methylphenyl, 2-methoxyphenyl, 3-methoxyphenyl, 4-methoxyphenyl, 2-ethoxyphenyl, 3-ethoxyphenyl, 2-hydroxyphenyl, 2,5-dimethoxyphenyl, 2-trifluoromethyoxyphenyl, 3-trifluoromethylphenyl, 2-(methylsulfonyl)phenyl, 3-(methylthio)phenyl, 4-(methoxyethoxy)phenyl, 4-(4-fluorobenzyloxy)phenyl, 4-(4-fluorophenoxy)phenyl, 3-cyclopropoxyphenyl, 3-(cyclopropylmethoxy)phenyl, and 2-(methylamino)phenyl;
4.82. Method 4, or any of 4.1-4.80, wherein in the Compound of Formula II, A is selected from the group consisting of: pyrid-4-yl, thiophen-2-yl, indol-1-yl, indol-3-yl, 5-fluoroindol-3-yl, indazol-1-yl, indazol-3-yl, indazol-7-yl, benzofuran-4-yl, benzofuran-7-yl, 2,3-dihydrobenzofuran-7-yl, 2-methylbenzofuran-7-yl, benzo[d]isoxazol-3-yl, benzo[d]isoxazol-4-yl, benzo[d]isoxazol-7-yl, 6-fluorobenzo[d]isoxazol-3-yl, benzo[d]isothiazol-3-yl, benzo[d]imidazole-1-yl, benzo[d][1,2,3]triazol-1-yl, isoquinolin-7-yl, quinolin-8-yl, quinoxaline-5-yl, quinazolin-7-yl, and 2-oxopyridin-1 (2H)-yl;
4.83. Method 4, or any of 4.1-4.80, wherein in the Compound of Formula II, A is selected from the group consisting of: phenyl, 2-ethylphenyl, 4-fluorophenyl, 2-methoxyphenyl, 3-methoxyphenyl, benzofuran-7-yl, benzo[d]isoxazol-3-yl, and benzo[d]isothiazol-3-yl;
4.84. Method 4, or any of 4.1-4.83, wherein in the Compound of Formula II, m is 1; 4.85. Method 4, or any of 4.1-4.83, wherein in the Compound of Formula II, m is 2;
4.86. Method 4, or any of 4.1-4.85, wherein in the Compound of Formula II, n is 2;
4.87. Method 4, or any of 4.1-4.85, wherein in the Compound of Formula II, n is 3;
4.88. Method 4, or any of 4.1-4.85, wherein in the Compound of Formula II, n is 4;
4.89. Method 4, or any of 4.1-4.85, wherein in the Compound of Formula II, n is 5;
4.90. Method 4, or any of 4.1-4.89, wherein in the Compound of Formula II, X is S, O, CH₂, NH, N(CH₃), or spiro-joined cyclopropyl; Y is CH₂, C(O), or spiro-joined cyclopropyl, and Z is a bond, —O—, —C(O)—, —O(CH₂)₂O—, or —C(═NOCH₃)—;
4.91. Method 4, or any of 4.1-4.89, wherein in the Compound of Formula II, X is N(CH₃) or spiro-joined cyclopropyl; Y is CH₂, C(O), or spiro-joined cyclopropyl, and Z is a bond, —O—, or —C(O)—;
4.92. Method 4, or any of 4.1-4.91, wherein the Compound of Formula II is a compound of Formula Ia:

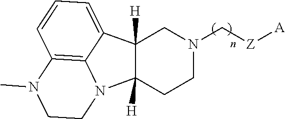

wherein n, Z, and A are defined as in any preceding embodiment;
4.93. Method 4, or any of 4.1-4.91, wherein the Compound of Formula II is a compound of Formula Ib:

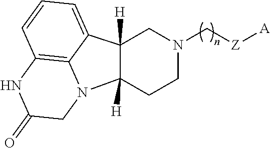

wherein n, Z, and A are defined as in any preceding embodiment;
4.94. Method 4, or any of 4.1-4.91, wherein the Compound of Formula II is a compound of Formula Ic:

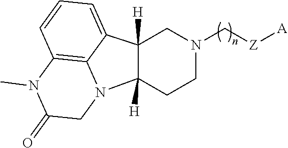

wherein n, Z, and A are defined as in any preceding embodiment;
4.95. Method 4, or any of 4.1-4.91, wherein the Compound of Formula II is a compound of Formula Id:

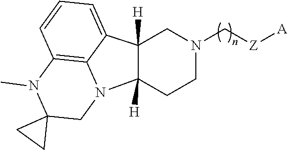

wherein n, Z, and A are defined as in any preceding embodiment;
4.96. Method 4, or any of 4.1-4.91, wherein the Compound of Formula II is a compound of Formula Ie:

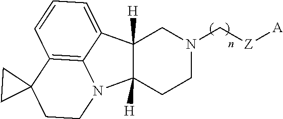

wherein n, Z, and A are defined as in any preceding embodiment;
4.97. Method 4, or any of 4.1-4.96, wherein in the Compound of Formula II, n is 4 and Z is a bond;
4.98. Method 4, or any of 4.1-4.96, wherein in the Compound of Formula II, n is 3 and Z is —O— or —C(O)—;
4.99. Method 4, or any of 4.1-4.96, wherein in the Compound of Formula II, n is 3 and Z is a bond;
4.100. Method 4, or any of 4.1-4.96, wherein in the Compound of Formula II, n is 2 and Z is —O— or —C(O)—;
4.101. Method 4, or any of 4.1-4.96, wherein in the Compound of Formula II, n is 2 and Z is a bond;
4.102. Method 4, or any of 4.1-4.96, wherein in the Compound of Formula II, n is 1 and Z is —O— or —C(O)—;
4.103. Method 4, or any of 4.1-4.96, wherein in the Compound of Formula II, n is 1 and Z is a bond;
4.104. Method 4, or any of 4.1-4.103, wherein in the Compound of Formula II, A is H or C₃₋₆cycloalkyl (e.g., cyclopropyl or cyclohexyl), Z is a bond or —C(O)—, and n is 1, 2, or 3;
4.105. Method 4, or any of 4.1-4.104, wherein the Compound of Formula II is selected from the group consisting of:

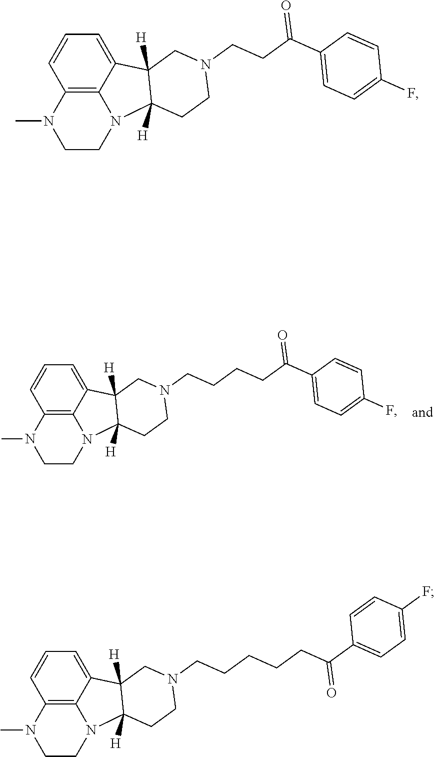 [SMILES: CN1CCN2c3c(cccc31)[C@@H]1CN(CCC(=O)c3ccc(F)cc3)CC[C@@H]12 ; CN1CCN2c3c(cccc31)[C@@H]1CN(CCCCC(=O)c3ccc(F)cc3)CC[C@@H]12 ; CN1CCN2c3c(cccc31)[C@@H]1CN(CCCCCC(=O)c3ccc(F)cc3)CC[C@@H]12]

each independently in free or pharmaceutically acceptable salt or form;
4.106. Method 4, or any of 4.1-4.105, wherein the Compound of Formula II is:

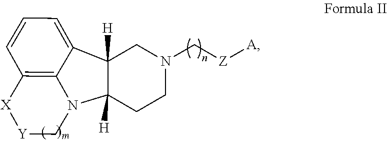

wherein the variables are defined as provided in any of the following embodiments:

| X | Y | m | n | Z | A |
|---|---|---|---|---|---|
| —N(CH₃)— | —CH₂— | 1 | 3 | —O— | 2-CN-4-F-phenyl |
| —N(CH₃)— | —CH₂— | 1 | 4 | —C(O)— | 4-F-phenyl |
| —N(CH₃)— | —CH₂— | 1 | 2 | —C(O)— | 4-F-phenyl |
| —N(CH₃)— | Cyp | 1 | 3 | —C(O)— | 4-F-phenyl |
| —N(CH₃)— | —CH₂— | 1 | 5 | —C(O)— | 4-F-phenyl |
| —N(CH₃)— | —CH₂— | 1 | 3 | —C(O)— | 4-(CH₃OCH₂CH₂O)-phenyl |
| Cyp | —CH₂— | 1 | 3 | —C(O)— | 4-F-phenyl |
| Cyp | —CH₂— | 1 | 3 | —O— | 4-F-phenyl |
| —NH— | —C(O)— | 1 | 3 | —O— | 3-Me-4-F-phenyl |
| —NH— | —C(O)— | 1 | 3 | —O— | 3-Cl-4-F-phenyl |
| —NH— | —C(O)— | 1 | 3 | —O— | 3-CN-4-F-phenyl |
| —N(CH₃)— | —CH₂— | 1 | 2 | —O(CH₂)₂O— | 4-F-phenyl |
| —N(CH₃)— | —CH₂— | 1 | 3 | —C(O)— | 4-((4-F-benzyl)oxy) |
| —N(CH₃)— | Cyp | 1 | 2 | —C(O)— | 4-F-phenyl |
| —N(CH₃)— | Cyp | 1 | 2 | —O— | 4-F-phenyl |
| —N(CH₃)— | Cyp | 1 | 2 | —C(O)— | 2-Me-4-F-phenyl |
| —N(CH₃)— | Cyp | 1 | 2 | —O— | 2-Me-4-F-phenyl |
| —NH— | —C(O)— | 1 | 2 | —O— | 4-F-phenyl |
| —N(CH₃)— | Cyp | 1 | 3 | bond | 3-Cl-phenyl |
| —N(CH₃)— | Cyp | 1 | 3 | bond | 3-MeO-phenyl |
| —N(CH₃)— | Cyp | 1 | 3 | bond | 3-CF₃-phenyl |
| —N(CH₃)— | Cyp | 1 | 3 | bond | 2-Cl-phenyl |
| —N(CH₃)— | Cyp | 1 | 3 | bond | 2-Me-phenyl |
| —N(CH₃)— | Cyp | 1 | 2 | bond | 2-Cl-phenyl |
| —N(CH₃)— | Cyp | 1 | 2 | bond | 2-MeO-phenyl |
| —N(CH₃)— | —CH₂— | 1 | 2 | —O— | 4-F-phenyl |
| —N(CH₃)— | —CH₂— | 1 | 2 | —O— | 2-MeO-4-F-phenyl |
| —N(CH₃)— | —CH₂— | 1 | 3 | bond | 2-MeO-phenyl |
| —N(CH₃)— | —CH₂— | 1 | 3 | bond | 3-MeO-phenyl |
| —N(CH₃)— | —CH₂— | 1 | 3 | bond | 3-Cl-phenyl |
| —N(CH₃)— | —CH₂— | 1 | 3 | bond | 2-Cl-phenyl |
| —N(CH₃)— | —CH₂— | 1 | 3 | bond | 3-CF₃-phenyl |
| —N(CH₃)— | —CH₂— | 1 | 2 | bond | 2-Cl-phenyl |
| —N(CH₃)— | —CH₂— | 1 | 2 | bond | 2-MeO-phenyl |
| —N(CH₃)— | —CH₂— | 1 | 3 | bond | 2-Me-phenyl |
| —CH₂— | —CH₂— | 2 | 2 | —C(O)— | 4-F-phenyl |
| —CH₂— | —CH₂— | 2 | 2 | —O— | 4-F-phenyl |
| —CH₂— | —CH₂— | 2 | 2 | bond | 4-F-phenyl |
| —N(CH₃)— | Cyp | 1 | 3 | bond | 2-MeO-phenyl |
| —N(CH₃)— | Cyp | 1 | 2 | bond | benzo[d]isoxazol-3-yl |
| —N(CH₃)— | —CH₂— | 1 | 2 | —C(O)— | 2-Me-4-F-phenyl |
| —O— | —CH₂— | 2 | 2 | —O— | 4-F-phenyl |
| —O— | —CH₂— | 2 | 2 | —C(O)— | 4-F-phenyl |
| —S— | —CH₂— | 2 | 2 | —C(O)— | 4-F-phenyl |
| —CH₂— | —CH₂— | 1 | 2 | —C(O)— | 4-F-phenyl |
| —S— | —CH₂— | 1 | 2 | —C(O)— | 4-F-phenyl |
| —NH— | Cyp | 1 | 2 | —O— | 4-F-phenyl |
| —NH— | Cyp | 1 | 2 | —O— | 2-Me-4-F-phenyl |
| —NH— | Cyp | 1 | 2 | —C(O)— | 4-F-phenyl |
| —NH— | Cyp | 1 | 2 | —C(O)— | 2-Me-4-F-phenyl |
| Cyp | —CH₂— | 1 | 2 | —O— | 4-F-phenyl |
| Cyp | —CH₂— | 1 | 2 | —O— | 2-Me-4-F-phenyl |
| Cyp | —CH₂— | 1 | 2 | —C(O)— | 4-F-phenyl |
| Cyp | —CH₂— | 1 | 2 | —C(O)— | 2-Me-4-F-phenyl |
| —NH— | —C(O)— | 1 | 2 | —C(O)— | 4-F-phenyl |
| —NH— | —C(O)— | 1 | 2 | —C(O)— | 2-Me-4-F-phenyl |
| —NH— | —C(O)— | 1 | 2 | —C(O)— | 2-F-4-Me-phenyl |
| —CH₂— | —CH₂— | 2 | 3 | bond | 2-MeO-phenyl |
| —CH₂— | —CH₂— | 2 | 3 | bond | 2-HO-phenyl |
| —CH₂— | —CH₂— | 2 | 3 | bond | 2-Cl-phenyl |
| —CH₂— | —CH₂— | 2 | 2 | bond | 2-Cl-phenyl |
| —CH₂— | —CH₂— | 2 | 3 | bond | 3-MeO-phenyl |
| —CH₂— | —CH₂— | 2 | 3 | bond | 3-CF₃-phenyl |
| —CH₂— | —CH₂— | 2 | 3 | bond | 3-Cl-phenyl |
| —N(CH₃)— | —CH₂— | 1 | 2 | bond | benzo[d]isoxazol-3-yl |
| —N(CH₃)— | Cyp | 1 | 2 | bond | 6-F-benzo[d]isoxazol-3-yl |
| —N(CH₃)— | —CH₂— | 1 | 2 | bond | 6-F-benzo[d]isoxazol-3-yl |
| Cyp | —CH₂— | 1 | 3 | —O— | 2-Me-4-F-phenyl |
| —N(CH₃)— | —CH₂— | 1 | 3 | —C(O)— | 4-(4-F-PhO)-phenyl |
| Cyp | —CH₂— | 1 | 2 | bond | 2-MeO-phenyl |
| —N(CH₃)— | —CH₂— | 1 | 2 | bond | 3-MeO-phenyl |
| Cyp | —CH₂— | 1 | 2 | bond | benzo[d]isoxazol-3-yl |
| —N(CH₃)— | Cyp | 1 | 4 | bond | 2-CN-phenyl |
| —N(CH₃)— | Cyp | 1 | 2 | bond | 2-CN-phenyl |
| —N(CH₃)— | —CH₂— | 1 | 2 | bond | 2-CN-phenyl |
| —N(CH₃)— | —CH₂— | 1 | 3 | bond | 2-CN-phenyl |
| —N(CH₃)— | —CH₂— | 1 | 2 | bond | 2-oxopyridin-1(2H)-yl |
| —N(CH₃)— | —CH₂— | 1 | 2 | bond | 2-MeO-4-F-phenyl |
| —N(CH₃)— | Cyp | 1 | 2 | bond | 2-MeO-4-F-phenyl |
| —N(CH₃)— | —CH₂— | 1 | 2 | bond | 2-CF₃O-phenyl |
| —N(CH₃)— | —CH₂— | 1 | 2 | bond | 2-Et-phenyl |
| —N(CH₃)— | —CH₂— | 1 | 2 | bond | 4-MeO-phenyl |
| —N(CH₃)— | —CH₂— | 1 | 2 | bond | 3-CN-phenyl |
| —N(CH₃)— | —CH₂— | 1 | 2 | bond | 4-CN-phenyl |
| —N(CH₃)— | —CH₂— | 1 | 2 | bond | 5-F-indol-3-yl |
| —N(CH₃)— | —CH₂— | 1 | 3 | —C(O)— | 2-MeO-phenyl |
| —N(CH₃)— | —C(O)— | 1 | 2 | —O— | 4-F-phenyl |
| —N(CH₃)— | —C(O)— | 1 | 2 | bond | 2-MeO-phenyl |
| —N(CH₃)— | —CH₂— | 1 | 2 | bond | 2-HO-phenyl |
| —N(CH₃)— | —CH₂— | 1 | 1 | bond | 2-MeO-phenyl |
| —N(CH₃)— | —CH₂— | 1 | 2 | bond | 2-EtO-phenyl |
| —N(CH₃)— | —CH₂— | 1 | 2 | bond | benzofuran-7-yl |
| —N(CH₃)— | —CH₂— | 1 | 3 | —O— | 2-MeO-phenyl |
| —N(CH₃)— | —CH₂— | 1 | 2 | bond | benzo[d]isothiazol-3-yl |
| —N(CH₃)— | —CH₂— | 1 | 2 | bond | 2,5-di-MeO-phenyl |
| —N(CH₃)— | —CH₂— | 1 | 1 | bond | 4-Et-phenyl |
| —N(CH₃)— | —CH₂— | 1 | 2 | bond | indol-3-yl |
| —N(CH₃)— | Cyp | 1 | 2 | bond | 2-Et-phenyl |
| —N(CH₃)— | Cyp | 1 | 2 | bond | 3-MeO-phenyl |
| —N(CH₃)— | —CH₂— | 1 | 1 | bond | phenyl |
| —N(CH₃)— | —CH₂— | 1 | 1 | bond | 3-MeO-phenyl |
| —N(CH₃)— | —CH₂— | 1 | 1 | bond | 3-Et-phenyl |
| —N(CH₃)— | —CH₂— | 1 | 1 | bond | 4-MeO-phenyl |
| —N(CH₃)— | —CH₂— | 1 | 1 | bond | 2-Et-phenyl |
| —N⁺(═O)⁻— | Cyp | 1 | 2 | —O— | 4-F-phenyl |
| —N(CH₃)— | —CH₂— | 1 | 2 | bond | 2-MeSO₂-phenyl |
| —N(CH₃)— | Cyp | 1 | 2 | bond | benzo[d]isothiazol-3-yl |
| —N(CH₃)— | —CH₂— | 1 | 1 | bond | cyclohexyl |
| —N(CH₃)— | —CH₂— | 1 | 2 | bond | 2-MeO-5-F-phenyl |
| —N(CH₃)— | —CH₂— | 1 | 2 | bond | 3-Et-Phenyl |
| —N(CH₃)— | —CH₂— | 1 | 2 | bond | 3-EtO-phenyl |
| —N(CH₃)— | Cyp | 1 | 2 | bond | benzofuran-7-yl |
| —N(CH₃)— | —CH₂— | 1 | 2 | bond | benzo[d]imidazole-1-yl |
| —N(CH₃)— | —CH₂— | 1 | 2 | bond | benzo[d][1,2,3]triazol-1-yl |
| —N(CH₃)— | —CH₂— | 1 | 2 | bond | indazol-3-yl |
| —NH— | —C(O)— | 1 | 2 | bond | benzo[d]isothiazol-3-yl |
| —N(CH₃)— | —C(O)— | 1 | 2 | bond | benzo[d]isothiazol-3-yl |
| —N(CH₃)— | Cyp | 1 | 1 | bond | 2-MeO-phenyl |
| —N(CH₃)— | —CH₂— | 1 | 1 | bond | benzo[d]isoxazol-3-yl |
| —N(CH₃)— | —CH₂— | 1 | 1 | bond | benzofuran-7-yl |
| —N(CH₃)— | —CH₂— | 1 | 1 | bond | benzo[d]isothiazol-3-yl |
| —N(CH₃)— | —CH₂— | 1 | 1 | bond | quinolin-8-yl |
| —N(CH₃)— | —CH₂— | 1 | 1 | bond | benzo[d]oxazol-7-yl |
| —N(CH₃)— | —CH₂— | 1 | 1 | bond | 2,3-dihydrobenzofuran-7-yl |
| —N(CH₃)— | —CH₂— | 1 | 2 | —O— | 2-CN-4-F-phenyl |
| —N(CH₃)— | —CH₂— | 1 | 2 | bond | indazol-1-yl |
| —N(CH₃)— | —C(O)— | 1 | 2 | —C(O)— | 4-F-phenyl |
| —N(CH₃)— | —CH₂— | 1 | 1 | bond | quinoxalin-5-yl |
| —N(CH₃)— | —CH₂— | 1 | 1 | —C(O)— | 4-F-Phenyl |
| —N(CH₃)— | —CH₂— | 1 | 2 | —C(═NOMe) | 4-F-Ph |
| —N(CH₃)— | —CH₂— | 1 | 3 | —C(O)— | phenyl |
| —N(CH₃)— | —CH₂— | 1 | 2 | —C(═NOMe) | phenyl |
| —N(CH₃)— | —C(O)— | 1 | 2 | —C(O)— | phenyl |
| —N(CH₃)— | —CH₂— | 1 | 2 | —C(O)— | thiophen-2-yl |
| —N(CH₃)— | —CH₂— | 1 | 1 | bond | 3-(O-cyclopropyl)phenyl |
| —N(CH₃)— | —CH₂— | 1 | 1 | bond | 3-(OCH₂-cyclopropyl)phenyl |
| —N(CH₃)— | —C(O)— | 1 | 2 | bond | 2-MeO-phenyl |
| —N(CH₃)— | —C(O)— | 1 | 2 | bond | 3-MeO-phenyl |
| —N(CH₃)— | —C(O)— | 1 | 2 | bond | benzo[d]isoxazol-3-yl |
| —N(CH₃)— | —CH₂— | 1 | 1 | bond | 3-MeS-phenyl |
| —N(CH₃)— | Cyp | 1 | 2 | —C(O)— | phenyl |
| —N(CH₃)— | Cyp | 1 | 3 | —C(OH)— | 4-F-phenyl |
| —N(CH₃)— | Cyp | 1 | 3 | —C(OH)— | phenyl |
| —N(CH₃)— | Cyp | 1 | 3 | —C(OMe)— | 4-F-phenyl |
| —N(CH₃)— | Cyp | 1 | 3 | —C(OMe)— | phenyl |
| —N(CH₃)— | —CH₂— | 1 | 2 | bond | 2-(MeNH)-phenyl |
| —N(CH₃)— | Cyp | 1 | 2 | bond | 2-HO-phenyl |
| —N(CH₃)— | Cyp | 1 | 2 | bond | 2-MeO-phenyl |
| —N(CH₃)— | Cyp | 1 | 2 | bond | 2-EtO-phenyl |
| —N(CH₃)— | Cyp | 1 | 2 | bond | 2-CF₃O-phenyl |
| —N(CH₃)— | Cyp | 1 | 2 | bond | 2-MeSO₂-phenyl |
| —N(CH₃)— | Cyp | 1 | 2 | bond | 2-(MeNH)-phenyl |
| —N(CH₃)— | Cyp | 1 | 2 | bond | 3-CN-phenyl |
| —N(CH₃)— | Cyp | 1 | 2 | bond | 4-MeO-phenyl |
| —N(CH₃)— | Cyp | 1 | 2 | bond | 4-CN-phenyl |
| —N(CH₃)— | —CH₂— | 1 | 1 | bond | quinazolin-7-yl |
| —N(CH₃)— | —CH₂— | 1 | 1 | bond | isoquinolin-7-yl |
| —N(CH₃)— | —CH₂— | 1 | 1 | bond | benzo[d]isoxazol-4-yl |
| —N(CH₃)— | —CH₂— | 1 | 1 | bond | benzofuran-4-yl |
| —N(CH₃)— | —CH₂— | 1 | 1 | bond | 2-Me-benzofuran-7-yl |
| —N(CH₃)— | —CH₂— | 1 | 2 | bond | 3-MeS-phenyl |
| —N(CH₃)— | —CH₂— | 1 | 2 | bond | 3-(O-Cyclopropyl)phenyl |
| —N(CH₃)— | —CH₂— | 1 | 2 | bond | 3-(OCH₂-Cyclopropyl)phenyl |
| —N(CH₃)— | —CH₂— | 1 | 2 | bond | benzo[d]isoxazol-4-yl |
| —N(CH₃)— | —CH₂— | 1 | 2 | bond | benzofuran-4-yl |
| —N(CH₃)— | —CH₂— | 1 | 2 | bond | 2-Me-benzofuran-7-yl |
| —N(CH₃)— | Cyp | 1 | 2 | bond | indazol-7-yl |
| —N(CH₃)— | Cyp | 1 | 2 | bond | quinolin-8-yl |
| —N(CH₃)— | Cyp | 1 | 2 | bond | pyrid-4-yl |
| —N(CH₃)— | —CH₂— | 1 | 2 | bond | quinolin-8-yl |
| —N(CH₃)— | —CH₂— | 1 | 2 | bond | indol-1-yl |
| —N(CH₃)— | —CH₂— | 1 | 2 | bond | pyrid-4-yl |
| —N(CH₃)— | Cyp | 1 | 1 | bond | 3-Et-phenyl |
| —N(CH₃)— | Cyp | 1 | 2 | —O— | 3-CN-4-F-phenyl |
| —N(CH₃)— | —CH₂— | 1 | 1 | bond | H |
| —N(CH₃)— | —CH₂— | 1 | 2 | bond | H |
| —N(CH₃)— | —CH₂— | 1 | 3 | bond | H |
| —N(CH₃)— | —CH₂— | 1 | 1 | bond | Cyclopentyl |
| —N(CH₃)— | —CH₂— | 1 | 1 | bond | Cyclobutyl |
| —N(CH₃)— | —CH₂— | 1 | 1 | bond | Cyclopropyl |

each independently in free or pharmaceutically acceptable salt or form, wherein Cyp refers to a spiro-joined cyclopropyl ring;
4.107. Method 2, or any of 2.1-2.105, wherein the Compound of Formula II is:

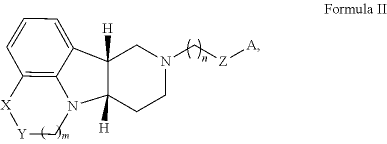

wherein the variables are defined as provided in any of the following embodiments:

| X | Y | m | n | Z | A |
|---|---|---|---|---|---|
| —N(CH₃)— | —CH₂— | 1 | 2 | —C(O)— | 4-F-phenyl |
| —N(CH₃)— | Cyp | 1 | 3 | —C(O)— | 4-F-phenyl |
| —N(CH₃)— | Cyp | 1 | 2 | —C(O)— | 4-F-phenyl |
| —N(CH₃)— | Cyp | 1 | 2 | —O— | 4-F-phenyl |
| —N(CH₃)— | Cyp | 1 | 2 | bond | 2-MeO-phenyl |
| —N(CH₃)— | Cyp | 1 | 2 | bond | benzo[d]isoxazol-3-yl |
| —N(CH₃)— | —CH₂— | 1 | 2 | bond | 3-MeO-phenyl |
| Cyp | —CH₂— | 1 | 2 | bond | benzo[d]isoxazol-3-yl |
| —N(CH₃)— | —CH₂— | 1 | 2 | bond | benzofuran-7-yl |
| —N(CH₃)— | —CH₂— | 1 | 2 | bond | benzo[d]isothiazol-3-yl |
| —N(CH₃)— | Cyp | 1 | 2 | bond | 2-Et-phenyl |
| —N(CH₃)— | Cyp | 1 | 2 | bond | 3-MeO-phenyl |
| —N(CH₃)— | Cyp | 1 | 2 | bond | benzo[d]isothiazol-3-yl |
| —N(CH₃)— | Cyp | 1 | 2 | bond | benzofuran-7-yl |
| —N(CH₃)— | —C(O)— | 1 | 2 | bond | benzo[d]isothiazol-3-yl |
| —N(CH₃)— | Cyp | 1 | 1 | bond | 2-MeO-phenyl |
| —N(CH₃)— | —C(O)— | 1 | 2 | —C(O)— | 4-F-phenyl |
| —N(CH₃)— | —C(O)— | 1 | 2 | bond | benzo[d]isoxazol-3-yl |
| —N(CH₃)— | Cyp | 1 | 2 | —C(O)— | Phenyl |
| —N(CH₃)— | Cyp | 1 | 1 | Bond | 3-Et-phenyl |

;

4.108. Method 4, or any of 4.1-4.107, wherein the Compound of Formula II is in free form;
4.109. Method 4, or any of 4.1-4.107, wherein the Compound of Formula II is in salt form, e.g., pharmaceutically acceptable salt form;
4.110. Method 4, or any of 4.1-4.107, wherein the Compound of Formula II is in acid addition salt form, for example, hydrochloric or toluenesulfonic acid salt form;
4.111. Method 4, or any of 4.1-4.110, wherein the Compound of Formula II is in substantially pure diastereomeric form (i.e., substantially free from other diastereomers);
4.112. Method 4 or any of 4.1-4.110, wherein the Compound of Formula II has a diastereomeric excess of greater than 70%, preferably greater than 80%, more preferably greater than 90% and most preferably greater than 95%;
4.113. Method 4 or any of 4.1-4.112, wherein the Compound of Formula II is in solid form, e.g., in crystal form;
4.114. Method 4 or any of 4.1-4.113, wherein the Compound of Formula II is in isolated or purified form (e.g., in at least 90% pure form, or at least 95% or at least 98% or at least 99%).
4.115. Method 4 or any of 4.1-4.114, wherein the Compound of Formula II has 5-HT_{2A} receptor binding affinity of at least 60% at 100 nM concentration, e.g., at least 70%, or at least 75%, or at least 80%, or at least 85%, or at least 90%, or at least 95%, or at least 98%, at 100 nM concentration;
4.116. Method 4 or any of 4.1-4.115, wherein the Compound of Formula II has a 5-HT_{2A} receptor dissociation constant (K_{d}) of less than 250 nM, or less than 100 nM, or less than 70 nM, or less than 60 nM, or less than 50 nM, or less than 40 nM, or less than 30 nM, or less than 20 nM, or less than 10 nM;
4.117. Method 4 or any of 4.1-4.116, wherein the Compound of Formula II is an agonist of beta-arrestin signaling via the 5-HT_{2A} receptor, e.g., a partial agonist or a full agonist;
4.118. Method 4.117, wherein the Compound of Formula II is a partial agonist of beta-arrestin signaling having an Eₘₐₓ of less than 90%, or less than 80%, or less than 70%, or less than 60%, or less than 50%, or less than 40%, or less than 30%, or less than 20%, or less than 10%, relative to a full agonist (e.g., alpha-methylserotonin);
4.119. Method 4.117 or 4.118, wherein the Compound of Formula II has an EC₅₀ for 5-HT_{2A} receptor beta-arrestin agonism of less than 500 nM, or less than 200 nM, or less than 150 nM, or less than 100 nM, or less than 70 nM, or less than 60 nM, or less than 50 nM, or less than 40 nM, or less than 30 nM, or less than 20 nM, or less than 10 nM;
4.120. Method 4.117, 4.118 or 4.119, wherein the Compound of Formula II has a beta-arrestin signaling relative intrinsic activity (RAᵢ) of less than 1.0 compared to the reference compound alpha-methylserotonin, e.g., a relative intrinsic activity of less than 0.8, or less than 0.6, or less than 0.5, or less than 0.4, or less than 0.3, or less than 0.2, or less than 0.1, or 0.1 to 0.8, or 0.2 to 0.8, or 0.4 to 0.8, or 0.5 to 0.8, or 0.2 to 0.6, or 0.2 to 0.5, or 0.2 to 0.4, or 0.5 to 1.0, or 0.5 to 0.9, or 0.5 to 0.8, or 0.6 to 0.9, or 0.6 to 0.8;
4.121. Method 4.117, 4.118, or 4.119, wherein the Compound of Formula II has a beta-arrestin signaling relative intrinsic activity (RAᵢ) of greater than 1.0 compared to the reference compound alpha-methylserotonin, e.g., a relative intrinsic activity of 1.0 to 1.2, or 1.0 to 1.4, or 1.0 to 1.6;
4.122. Method 4 or any of 4.1-4.116, wherein the Compound of Formula II is an antagonist of beta-arrestin signaling via the 5-HT_{2A} receptor;
4.123. Method 4.122, wherein the Compound of Formula II has an IC₅₀ for 5-HT_{2A} receptor beta-arrestin antagonism of less than 300 nM, or less than 200 nM, or less than 100 nM, or less than 70 nM, or less than 60 nM, or less than 50 nM, or less than 40 nM, or less than 30 nM, or less than 20 nM, or less than 10 nM;
4.124. Method 4 or any of 4.1-4.116, wherein the Compound of Formula II is not an antagonist of beta-arrestin signaling via the 5-HT_{2A} receptor;
4.125. Method 4.124, wherein the Compound of Formula II has an IC₅₀ for 5-HT_{2A} receptor beta-arrestin antagonism of greater than 10 nM, or greater than 50 nM, or greater than 100 nM, or greater than 250 nM, or greater than 500 nM, or greater than 1000 nM, or greater than 5000 nM, or greater than 10,000 nM;
4.126. Method 4 or any of 4.1-4.125, wherein the Compound of Formula II is not an agonist of G-q signaling via the 5-HT_{2A} receptor, or is a weak agonist thereof;
4.127. Method 4.126, wherein the Compound of Formula II is a partial agonist of G-q signaling having an Eₘₐₓ of less than 90%, or less than 80%, or less than 70%, or less than 60%, or less than 50%, or less than 40%, or less than 30%, or less than 20%, or less than 10%, relative to a full agonist (e.g., alpha-methylserotonin), preferably an Eₘₐₓ of less than 50% or less than 30% or less than 10%;
4.128. Method 4.126 or 4.127, wherein the Compound of Formula II has an EC₅₀ for 5-HT_{2A} receptor G-q agonism of greater than 10 nM, or greater than 25 nM, or greater than 50 nM, or greater than 100 nM, or greater than 150 nM, or greater than 200 nM, or greater than 500 nM, or greater than 1000 nM, or greater than 2000 nM, or greater than 5000 nM, or greater than 10,000 nM;
4.129. Method 4.126, 4.127 or 4.128, wherein the Compound of Formula II has a G-q signaling relative intrinsic activity (RAᵢ) of less than 1.0 compared to the reference compound alpha-methylserotonin, e.g., a relative intrinsic activity of less than 0.8, or less than 0.6, or less than 0.5, or less than 0.4, or less than 0.3, or less than 0.2, or less than 0.1, or 0.1 to 0.8, or 0.2 to 0.8, or 0.4 to 0.8, or 0.5 to 0.8, or 0.2 to 0.6, or 0.2 to 0.5, or 0.2 to 0.4, or 0.5 to 1.0, or 0.5 to 0.9, or 0.5 to 0.8, or 0.6 to 0.9, or 0.6 to 0.8;
4.130. Method 4 or any of 4.1-4.129, wherein the Compound of Formula II is an antagonist of G-q signaling via the 5-HT_{2A} receptor;
4.131. Method 4.130, wherein the Compound of Formula II has an IC₅₀ for 5-HT_{2A} receptor G-q antagonism of less than 10 nM, or less than 25 nM, or less than 50 nM, or less than 100 nM, or less than 150 nM, or less than 200 nM, or less than 500 nM;
4.132. Method 4 or any of 4.1-4.131, wherein the Compound of Formula II has a bias ratio (beta-arrestin/G-q) for agonism 5-HT_{2A} receptor of at least 2, or at least 5, or at least 10, or at least 25, or at least 50, or at least 100, or at least 150, or at least 200 or at least 500, or at least 1000, or at least 10,000, or undefined (i.e., where the compound has any degree of beta-arrestin agonism and zero G-q agonism);
4.133. Method 4 or any of 4.1-4.132, wherein the Compound of Formula II is an antagonist or agonist of the D1 and/or D2 dopamine receptor (e.g., having at least 70% receptor affinity at 100 nM concentration or an IC₅₀ of less than 100 nM);
4.134. Method 4 or any of 4.1-4.132, wherein the Compound of Formula II is not active at the D1 and/or D2 dopamine receptor (e.g., having less than 50% receptor affinity at 100 nM concentration and/or an EC50 or IC₅₀ of more than 500 nM);
4.135. Method 4 or any of 4.1-4.134, wherein the Compound of Formula II is an antagonist of the serotonin transporter (e.g., having at least 70% receptor binding affinity at 100 nM concentration or an IC₅₀ of less than 100 nM);
4.136. Method 4 or any of 4.1-4.134, wherein the Compound of Formula II is not active at the serotonin transporter (e.g., having less than 50% receptor binding affinity at 100 nM concentration and/or an EC₅₀ or IC₅₀ of more than 500 nM);
4.137. Method 4 or any of 4.1-4.136, wherein the Compound of Formula II is an agonist, antagonist, or partial agonist of the mu-opioid receptor (e.g., having at least 70% receptor binding affinity at 100 nM concentration or an EC₅₀ or IC₅₀ of less than 100 nM);
4.138. Method 4 or any of 4.1-4.136, wherein the Compound of Formula II is not active at the mu-opioid receptor (e.g., having at less than 50% receptor binding affinity at 100 nM concentration and/or an EC₅₀ or IC₅₀ of more than 500 nM);
4.139. Method 4, or any of 4.1-4.138, wherein the Compound of Formula II is non-hallucinogenic, e.g., at therapeutic doses for the treatment of a neuropsychiatric disorder described herein (e.g., depression, anxiety, etc.) the compound does not cause visual or auditory hallucinations, visual distortions (such as drifting, morphing, breathing or melting of objects and surfaces in the field of view), detachment from reality, dissociation, delirium, undesired altered states of consciousness;
4.140. Method 4, or any of 4.1-4.139, wherein the Compound of Formula II does not stimulate head twitch response in an animal test model, or is antagonist of DOI-induced head twitch response;
4.141. Method 4, or any of 4.1-4.140, wherein the Compound of Formula II is effective in a murine model of depression (tail suspension or forced swim test);
4.142. Method 4, or any of 4.1-4.141, wherein the Compound of Formula II is effective in an animal model of social anxiety disorder or anhedonia;
4.143. Method 4, or any of 4.1-4.142, wherein the Compound of Formula II does not have 5-HT_{2B} agonist activity (e.g., an EC50 of greater than 100 nM, or greater than 500 nM, or greater than 1000 nM, or greater than 10,000 nM);
4.144. Method 4, or any of 4.1-4.143, wherein the Compound of Formula II has 5-HT_{2B} antagonist activity (e.g., an IC₅₀ of less than 1000 nM, or less than 500 nM, or less than 250 nM, or less than 100 nM, or less than 50 nM, or less than 25 nM, or less than 15 nM);
4.145. Method 4, or any of 4.1-4.144, wherein the Compound of Formula II has 5-HT_{2C} agonist activity (e.g., an EC50 of less than 1000 nM, or less than 500 nM, or less than 250 nM, or less than 100 nM, or less than 50 nM, or less than 25 nM, or less than 15 nM);
4.146. Method 4, or any of 4.1-4.144, wherein the Compound of Formula II does not have 5-HT_{2C} antagonist activity (e.g., an IC₅₀ of greater than 100 nM, or greater than 500 nM, or greater than 1000 nM, or greater than 10,000 nM);
4.147. Method 4, or any of 4.1-4.146, wherein the Compound of Formula II binds to the alpha-1A adrenergic receptor (e.g., with a binding affinity Ki of less than 1000 nM, or less than 500 nM, or less than 250 nM, or less than 200 nM, or less than 150 nM, or less than 100 nM, or less than 50 nM, or less than 25 nM);
4.148. Method 4, or any of 4.1-4.147, wherein the Compound of Formula II does not cause psychoses (e.g., prolonged or intermittent psychoses);
4.149. Method 4, or any of 4.1-4.148, wherein the Compound of Formula II does not promote self-harm or harm to others in the patient;
4.150. Method 4, or any of 4.1-4.149, wherein the Compound of Formula II does not cause heart valvulopathy or pulmonary arterial hypertension, e.g., wherein the compound is safe to administer to a patient having cardiac comorbidities;
4.151. Method 4, or any of 4.1-4.150, wherein the Compound of Formula II does not cause abuse or dependence (e.g., physical or psychological dependence);
4.152. Method 4, or any of 4.1-4.151, wherein the Compound of Formula II is functionally inactive at one or more of the following receptors and ion channels: adenosine A2A, alpha-1A adrenergic, alpha-2A adrenergic, beta-1 adrenergic, beta-2 adrenergic, GABA-A benzodiazepine site (BZD, central), CB1 cannabinoid, CB2 cannabinoid, cholecystokinin CCK1, endothelin-A (ETA), NMDA, histamine H1, histamine H2, MAO-A, muscarinic M1, muscarinic M2, muscarinic M3, nicotinic acetylcholine (neuronal alpha-4-beta-2), delta opioid, kappa opioid, mu opioid, serotonin-1A, serotonin-1B, serotonin-3, glucocorticoid (GR), androgen (AR), vasopressin VIA, cardiac calcium channel (dihydropyridine site), hERG potassium channel, voltage-gated potassium channel Kv, sodium channel (site 2), norepinephrine transporter, dopamine transporter, and/or serotonin transporter;
4.153. Method 4.152, wherein the Compound of Formula II has an in vitro receptor activity (for agonism or antagonism) of less than 60% inhibition of radioligand binding (e.g., at 100 nM test concentration) for any one or more of said receptor or ion channels, e.g., less than 50%, less than 40%, less than 30%, less than 20%, or less than 10%;
4.154. Method 4, or any of 4.1-4.153, wherein the Compound of Formula II is orally bioavailable (e.g., oral bioavailability of at least 10%, or at least 15%, or at least 20%, or at least 30%, or at least 40%);
4.155. Any foregoing method, wherein the method provides enhanced neural growth in the brain of the subject (e.g., in the prefrontal cortex region of the brain);
4.156. Any foregoing method, wherein the method provides enhanced neural connectivity, in the brain of the subject (e.g., in the prefrontal cortex region of the brain);
4.157. Any foregoing method, wherein the method provides increased synaptic density, in the brain of the subject (e.g., in the prefrontal cortex region of the brain);
4.158. Any foregoing method, wherein the method provides increased dendritic spine density, in the brain of the subject (e.g., in the prefrontal cortex region of the brain);
4.159. Any foregoing method, wherein the method provides increased dendritic spine lengths), in the brain of the subject (e.g., in the prefrontal cortex region of the brain);
4.160. Any foregoing method, wherein the method provides increased excitatory neurotransmission (e.g., enhanced glutamatergic transmission or increased rate of mEPSCs), in the brain of the subject (e.g., in the prefrontal cortex region of the brain);
4.161. Method 4, or any of 4.1-4.160, wherein the method provides the enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the medial prefrontal cortex region of the brain) within less than 4 weeks of the initiation of administration of the Compound of Formula II, e.g., less than 3 weeks, less than 2 weeks, less than 1 week, less than 6 days, less than 5 days, less than 4 days, less than 3 days, or less than 2 days, after the initiation of treatment with the Compound of Formula II;
4.162. Method 4, or any of 4.1-4.161, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the Compound of Formula II, e.g., for at least 3 weeks, or at least 4 weeks, or at least 2 months, or at least 3 months;
4.163. Method 4, or any of 4.1-4.162, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the Compound of Formula II, e.g., for at least 3 weeks, or at least 4 weeks, or at least 2 months, or at least 3 months, such as by measured by in vivo imaging (e.g., MRI);
4.164. Method 4, or any of 4.1-4.163, wherein the enhanced neural growth, enhanced neural connectivity, increased synaptic density, increased dendritic spine density, increased dendritic spine size, and/or increased excitatory neurotransmission is caused by the enhanced neuritogenesis and/or enhanced neurite outgrowth, and/or is characterized by or associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
4.165. Method 4, or any of 4.1-4.164, wherein the method enhances neuritogenesis and/or enhances neurite outgrowth within less than 4 weeks of the initiation of administration of the Compound of Formula II, e.g., less than 3 weeks, less than 2 weeks, less than 1 week, less than 6 days, less than 5 days, less than 4 days, less than 3 days, or less than 2 days, after the initiation of treatment with the Compound of Formula II;
4.166. Method 4, or any of 4.1-4.165, wherein the enhanced neuritogenesis or enhanced neurite outgrowth is characterized by increases in one or more of: the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization (e.g., measured or estimated using a Sholl Analysis);
4.167. Any foregoing method, wherein the method does not cause hallucinogenic side effects;
4.168. Any foregoing method, wherein the Compound of Formula II is administered in a daily dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base;
4.169. Any foregoing method, wherein the Compound of Formula II is administered in a dose equivalent to 1 to 100 mg of free base, e.g., 1 to 75 mg, or 1 to 60 mg, or 1 to 50 mg, or 1 to 40 mg, or 1 to 30 mg, or 1 to 20 mg, or 1 to 10 mg, of free base, at a frequency of every other day, or every two days, or every three days, or every four days, or every five days, or every six days, or every seven days;
4.170. Method 4, or any of 4.1-4.169, wherein the Compound of Formula II is administered as a unit dosage form for oral administration (e.g., enteral), for example, a tablet or capsule;
4.171. Method 4.170, wherein the unit dosage for oral administration (e.g., enteral), for example a tablet or capsule, comprises the Compound of Formula II in an amount equivalent 1 to 100 mg of free base, e.g., 1 to 75 mg, or 1 to 60 mg, or 1 to 40 mg, or 1 to 20 mg, or 1 to 10 mg, of free base, and a pharmaceutically acceptable diluent or carrier;
4.172. Method 4, or any of 4.1-4.171, wherein the Compound of Formula II is administered as a unit dosage for subcutaneous or transmucosal administration, e.g., a sublingual or buccal orally disintegrating tablet or film;
4.173. Method 4.172, wherein the unit dosage for subcutaneous or transmucosal administration, e.g., a sublingual or buccal orally disintegrating tablet or film, comprises the Compound of Formula II in an amount equivalent to 0.5 to 30 mg of free base, e.g., 1-10 mg of free base, and a pharmaceutically acceptable diluent or carrier;
4.174. Method 4, or any of 4.1-4.173, wherein the Compound of Formula II is administered as a long-acting injectable (LAI) composition, e.g., for intramuscular or subcutaneous injection;
4.175. Method 4.174, wherein the dose of the LAI composition is sufficient to provide the equivalent of a daily dose of 1 to 100 mg of free base of the Compound of Formula II, e.g., 1 to 75 mg, or 1 to 60 mg, or 1 to 40 mg, or 1 to 20 mg, or 1 to 10 mg, of free base, released over a period of time ranging from about 1 week to about 3 months, e.g., about 1 week to about 8 weeks, or about 1 week to about 6 weeks, or about 1 week to about 4 weeks, or about 1 week to about 3 weeks, or about 1 week to about 2 weeks;
4.176. Method 4.174 or 4.175, wherein the LAI composition comprises the Compound of Formula II dissolved, dispersed, suspended, or encapsulated in a polymeric matrix;
4.177. Method 4.176, wherein the polymeric matrix comprises one or more biocompatible and biodegradable polymers as defined herein, e.g., poly(hydroxycarboxylic acids), poly(amino acids), cellulose polymers, modified cellulose polymers, polyamides, and polyesters;
4.178. Method 4.177, wherein the one or more polymers comprises polylactic acid, polyglycolic acid, polycitric acid, polymalic acid, poly-beta-hydroxybutyric acid, poly(lactic acid-glycolic acid) copolymer, 2-hydroxybutyric acid-glycolic acid copolymer, polylactic acid-polyethylene glycol copolymer, polyglycolic acid-polyethylene glycol copolymer, poly(alkyl alpha-cyanoacrylate) such as poly(butyl cyanoacrylate) or poly(2-octyl cyanoacrylate), poly(ortho ester), polycarbonate, polyortho-carbonate, a polyamino acid, (for example poly-gamma.-L-alanine, poly-.gamma.-benzyl-L-glutamic acid or poly-y-methyl-L-glutamic acid), and/or hyaluronic acid ester;
4.179. Method 4.177, wherein the one or more polymers comprises polylactic acid, polyglycolic acid, polycitric acid, polymalic acid, or a poly(lactic acid-glycolic acid) copolymer;
4.180. Method 4.177, wherein the one or more polymers comprises a poly(lactic acid-glycolic acid) copolymer, e.g., poly-d,l-lactide-co-glycolide;
4.181. Any foregoing method, wherein the subject is an animal;
4.182. Any foregoing method, wherein the subject is a human (e.g., a patient suffering from a neuropsychiatric disorder);
4.183. Method 4.182, wherein the subject is a patient suffering from anxiety or depression, e.g., bipolar depression, major depressive disorder (MDD), post-traumatic stress disorder, or treatment-resistant depression;
4.184. Method 4.183, wherein the subject is a patient suffering from treatment resistant depression (e.g., depression which has not responded to treatment with an antidepressant agent selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof); 4.185. Method 4.184, wherein the subject is a patient suffering from bipolar depression or major depressive disorder;
4.186. Any foregoing method, wherein the method further comprises the concurrent administration of an anti-depressant agent (e.g., selected from a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor), a serotonin receptor antagonist, or any combination thereof), e.g., administered simultaneously, separately or sequentially;
4.187. Any foregoing method, wherein the method further comprises the concurrent administration of an NMDA receptor antagonist, for example, selected from ketamine (e.g., S-ketamine and/or R-ketamine), hydroxynorketamine, memantine, dextromethorphan, dextroallorphan, dextrorphan, amantadine, and agmatine, or any combination thereof, e.g., administered simultaneously, separately or sequentially;
4.188. Any foregoing method, wherein the method further comprises the concurrent administration of a NMDA receptor allosteric modulator, e.g., a NMDA receptor glycine-site modulator, such as rapastinel, nebostinel, apimostinel, D-cycloserine, or any combination thereof, e.g., administered simultaneously, separately or sequentially;
4.189. Any foregoing method, wherein the subject has previously been treated with but has not responded to, or has not responded adequately to, or who suffers undesirable side effects from, treatment with another antidepressant agent, for example, any one or more of a selective serotonin reuptake inhibitor (SSRI), a serotonin reuptake inhibitor (SRI), a tricyclic antidepressant, a monoamine oxidase inhibitor, a norepinephrine reuptake inhibitor (NRI), a dopamine reuptake inhibitor (DRI), an SRI/NRI, an SRI/DRI, an NRI/DRI, an SRI/NRI/DRI (triple reuptake inhibitor, or a serotonin receptor antagonist;
4.190. Any foregoing method, wherein the Compound of Formula II is administered as monotherapy, e.g., it is not administered concurrently or in conjunction with an anti-depressant, anti-psychotic, or anti-anxiety agent;
4.191. Any foregoing method, wherein the Compound of Formula II is administered without the direct supervision of a health care professional (e.g., the compound is self-administered by the subject (e.g., patient));
4.192. Any foregoing method, wherein the method does not comprise supervision or observation of the subject (e.g., patient) by a health care professional during or after (e.g., within 2 hours after) administration of a dose of the Compound of Formula II;
4.193. Any foregoing method, wherein the method does not put the subject (e.g., patient) at risk for sedation, dissociation, abuse, misuse, or suicidal ideation;
4.194. Any foregoing method, wherein the method does not result in hypertension (e.g., systolic and/or diastolic hypertension) within four hours after administration of a dose of the Compound of Formula II, e.g., an increase of more than 10 mm Hg, or more than 20 mm Hg, or more than 30 mm Hg, or more than 40 mm Hg, in systolic and/or diastolic blood pressure within 30 minutes to 4 hours after said dose;
4.195. Any foregoing method, wherein the method does not result in cognitive decline in the subject (e.g., patient);
4.196. Any foregoing method, wherein the subject (e.g., patient) has been diagnosed with or is at risk of aneurysmal vascular disease (e.g., thoracic aorta, abdominal aorta, intracranial, or peripheral arterial aneurysms), arteriovenous malformation or intracerebral hemorrhage;
4.197. Any foregoing method, wherein the subject (e.g., patient) is under concurrent treatment with an oral antidepressant selected from duloxetine, escitalopram, sertraline, or venlafaxine;
4.198. Any foregoing method, wherein the subject (e.g., patient) is not under concurrent treatment with an oral antidepressant selected from duloxetine, escitalopram, sertraline, or venlafaxine;
4.199. Any foregoing method, wherein the subject (e.g., patient) is unresponsive to, or cannot be treated with ketamine (e.g., S-ketamine), e.g., because it is contraindicated in said subject (e.g., patient);
4.200. Any foregoing method, wherein the enhanced neuritogenesis and/or enhanced neurite outgrowth is associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density.

In another aspect, the disclosure provides a Compound of Formula I or a deuterated analog thereof, as hereinbefore described, or a Compound of Formula II, as hereinbefore described, each in free or pharmaceutically acceptable salt form, for use in enhancing neuritogenesis and/or neurite outgrowth, in the brain of a subject in need thereof, e.g., in the prefrontal cortex region of the brain, e.g., for use in any of Methods 3 et seq, or 4, et seq.

In another aspect, the disclosure provides the use of a Compound of Formula I or a deuterated analog thereof, as hereinbefore described, or a Compound of Formula II, as hereinbefore described, each in free or pharmaceutically acceptable salt form, in the manufacture of a medicament for enhancing neuritogenesis and/or neurite outgrowth, in the brain of a subject in need thereof, e.g., in the medial prefrontal cortex region of the brain, e.g., for any of Methods 3, et seq, or 4 et seq.

In a third aspect, the present disclosure provides a method (Method 5) for enhancing neural growth, enhancing neuritogenesis, enhancing neurite outgrowth, enhancing neural connectivity, increasing synaptic density, increasing dendritic spine density, increasing dendritic spine size, and/or increasing excitatory neurotransmission, in a neural cell or neural tissue (e.g., a cell culture), in vitro or in vivo, the method comprising the step of contacting the neural cell or neural tissue with an effective amount of a Compound of Formula I or a deuterated analog thereof, or a Compound of Formula II, each in free or pharmaceutically acceptable salt form.

In further embodiments of the third aspect, the present disclosure provides:
5.1. Method 5, wherein the neural cell or neural tissue is contacted by an effective amount of a Compound of Formula I, or deuterated analog thereof, as described in any of Method 1 or 1.1-1.16 hereinabove;
5.2. Method 5, wherein the neural cell or neural tissue is contacted by an effective amount of a Compound of Formula II, as described in any of Method 2 or 2.1-2.154 hereinabove;
5.3. Any foregoing method, wherein the method provides enhanced neural growth in the neural cell or neural tissue;
5.4. Any foregoing method, wherein the method provides enhanced neural connectivity, in the neural cell or neural tissue;
5.5. Any foregoing method, wherein the method provides increased synaptic density, in the neural cell or neural tissue;
5.6. Any foregoing method, wherein the method provides increased dendritic spine density, in neural cell or neural tissue;
5.7. Any foregoing method, wherein the method provides increased dendritic spine size (e.g., increased width of spine heads and/or increased spine protrusion lengths), neural cell or neural tissue;
5.8. Any foregoing method, wherein the method provides increased excitatory neurotransmission (e.g., enhanced glutamatergic transmission or increased rate of mEPSCs), in the neural cell or neural tissue;
5.9. Method 5, or any of 5.1-5.8, wherein the method provides the enhanced neural growth, enhanced neural connectivity, increased synaptic density, increased dendritic spine density, increased dendritic spine size, and/or increased excitatory neurotransmission, in the neural cell or neural tissue within less than 4 weeks of the initiation of administration of the Compound of Formula I, or deuterated analog thereof, or Compound of Formula II, e.g., less than 3 weeks, less than 2 weeks, less than 1 week, less than 6 days, less than 5 days, less than 4 days, less than 3 days, or less than 2 days, after the initiation of treatment with the Compound of Formula I, or deuterated analog thereof, or Compound of Formula II;
5.10. Method 5, or any of 5.1-5.9, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the Compound of Formula I or deuterated analog thereof, or Compound of Formula II, e.g., for at least 3 weeks, or at least 4 weeks, or at least 2 months, or at least 3 months;
5.11. Method 5, or any of 5.1-5.10, wherein the method maintains at least 50% of the peak enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the Compound of Formula I or deuterated analog thereof, e.g., for at least 3 weeks, or at least 4 weeks, or at least 2 months, or at least 3 months, such as by measured by in vivo imaging (e.g., MRI);
5.12. Method 5, or any of 5.1-5.11, wherein the enhanced neural growth, enhanced neural connectivity, increased synaptic density, increased dendritic spine density, increased dendritic spine size, and/or increased excitatory neurotransmission is characterized by or caused by enhanced neuritogenesis and/or enhanced neurite outgrowth, and/or is characterized by or is associated with changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density;
5.13. Method 5, or any of 5.1-5.12, wherein the method provides enhanced neuritogenesis or enhanced neurite outgrowth in the neural cell or neural tissue;
5.14. Method 5, or any of 5.1-5.13, wherein the method enhances neuritogenesis and/or enhances neurite outgrowth within less than 4 weeks of the initiation of administration of the Compound of Formula I or deuterated analog thereof, or the Compound of Formula II, e.g., less than 3 weeks, less than 2 weeks, less than 1 week, less than 6 days, less than 5 days, less than 4 days, less than 3 days, or less than 2 days, after the initiation of treatment with the Compound of Formula I or deuterated analog thereof, or Compound of Formula II;
5.15. Method 5, or any of 5.1-5.14, wherein the enhanced neuritogenesis or enhanced neurite outgrowth is characterized by increases in one or more of: the total number of neurites per neuron, the length of neurites (individually and/or in total for a neuron), the number of branch points on neurites, the number of neurite roots, the number of neurite nodes, the total number of neurite extremities, the total length of neurons (with or without branches), and total neurite arborization (e.g., measured or estimated using a Sholl Analysis);
5.16. Method 5, or any of 5.1-5.15, wherein the method provides or causes changes in synaptic neurotransmitter receptor or neurotransmitter transporter density (e.g., increases or decreases in such receptor or transporter density), such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density
5.17. Method 5, or any of 5.1-5.16, wherein the neural cell or neural tissue is a cell culture (e.g., of pluripotent stem cells, neural progenitor cells, immature neurons, or mature neurons);
5.18. Method 5.17, wherein the neural cell or neural tissue is of human origin (e.g., an immortalized human cell line);
5.19. Method 5.17, wherein the neural cell or neural tissue is of animal origin (e.g., murine cell line);
5.20. Method 5, or any of 5.1-5.16, wherein the neural cell or neural tissue is isolated from an animal after expiration of the animal (e.g., wherein the compound is administered to the animal live and the animal is expired and dissected to obtain the neural cell or neural tissue for post-mortem analysis);
5.21. Method 5.20, wherein the compound does not cause observations consistent with hallucinogenic side effects in the animal during administration of the compound;
5.22. Method 5, or any of 5.1-5.21, wherein the method is carried out to determine whether the compound is effective in enhancing neural growth, enhancing neuritogenesis, enhancing neurite outgrowth, enhancing neural connectivity, increasing synaptic density, increasing dendritic spine density, increasing dendritic spine size, and/or increasing excitatory neurotransmission in vivo in an animal model, e.g. an animal model of a neuropsychiatric disorder;
5.23. Any of Methods 5.19-5.22, wherein the animal is an animal model of a neuropsychiatric disorder, e.g., of anxiety, depression, psychosis, or a neurodegenerative disorder.

In another aspect, the disclosure provides a Compound of Formula I or a deuterated analog thereof, as hereinbefore described, or a Compound of Formula II, as hereinbefore described, each in free or pharmaceutically acceptable salt form, for use in a method for enhancing neural growth, enhancing neuritogenesis, enhancing neurite outgrowth, enhancing neural connectivity, increasing synaptic density, increasing dendritic spine density, increasing dendritic spine size, and/or increasing excitatory neurotransmission, in a neural cell or neural tissue (e.g., a cell culture), in vitro or in vivo, e.g., any of Method 5 et seq.

As used hereinbelow, the “Compounds of the Disclosure” refers to any compound described in any of Methods I or 1.1-1.16 or Methods 2 or 2.1-2.154, such as lumateperone, related analogs thereof, deuterated analogs thereof, and other octahydro-1H-pyrido[3′,4′: 4,5]pyrrolo[1,2,3-de]quinoxalines, each in free or pharmaceutically acceptable salt form.

As used herein, the term “spiro-joined” is meant to clarify that the stated C₃₋₆cycloalkyl group or 3-6-membered heterocycloalkyl is present in a spiro-junction, meaning that one atom of said cyclic group is an atom of the ring to which the group is attached. For example, the follow are examples of compounds of Formula II having spiro-joined cyclic groups within the scope of the present disclosure:

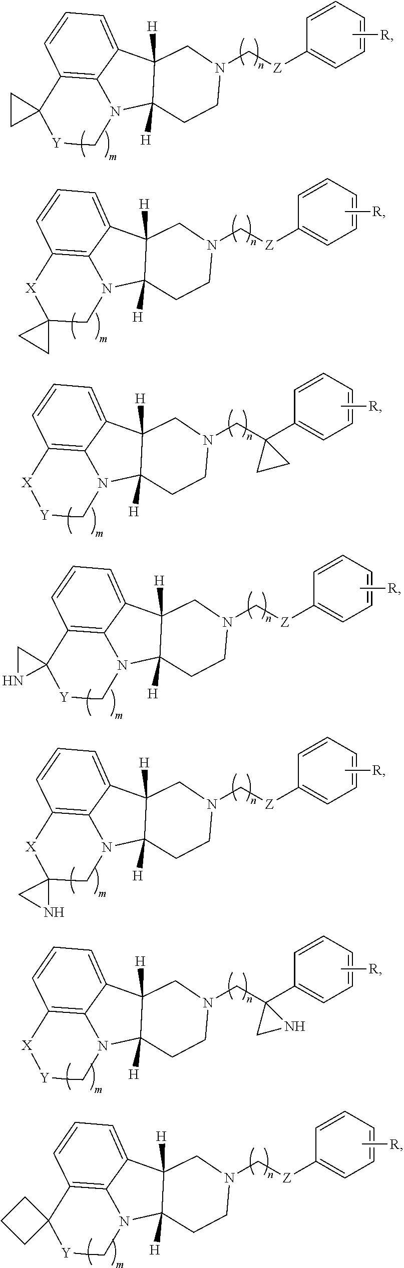

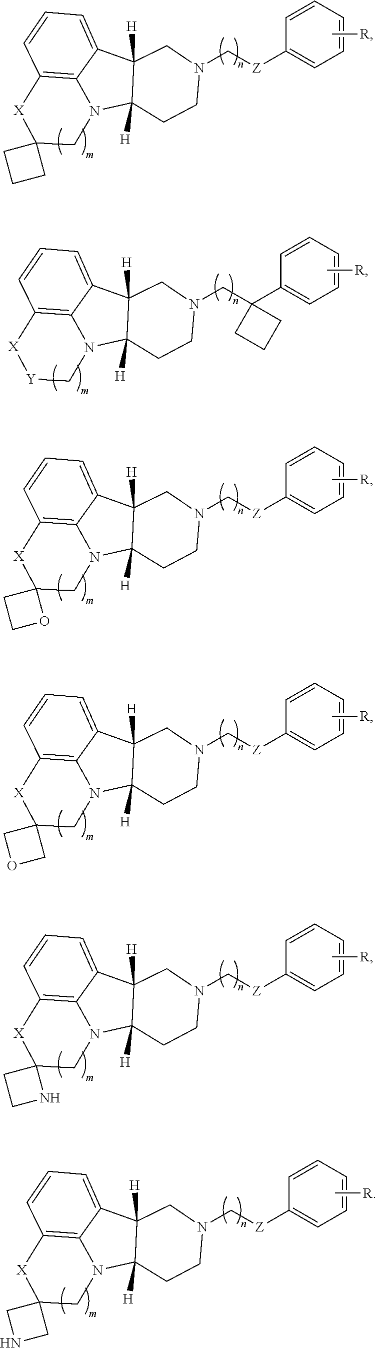

In each of the above examples, the cyclopropane, cyclobutane, aziridine, azetidine, or oxetane, may be replaced by any other C₃₋₆cycloalkyl or 3-6-membered heterocycloalkyl, including, but not limited to, cyclopentane, cyclohexane, tetrahydrofuran, tetrahydropyran, pyrrolidine, piperidine, piperazine, or morpholine.

The Compounds of Formula II are biased agonists of the serotonin 5-HT_{2A} receptor. The term “biased agonist” as used herein, is used in reference to a compound having activity at the serotonin 5-HT_{2A} receptor with either partial or full agonism for beta-arrestin signaling via the receptor, but with either antagonism or weak partial agonism for G-q mediated signaling. A useful measure of bias is the “bias ratio”, which is calculated as the ratio of the intrinsic relative activity (RAᵢ) for beta-arrestin signaling over the RAᵢ for G-q signaling. A non-biased agonist has a bias ratio of 1.0. A biased agonist has a non-zero bias ratio. In some embodiments, compounds of the present disclosure are preferably biased towards beta-arrestin signaling, and thus have a bias ratio greater than 1.0. More preferably, the bias ratio towards beta-arrestin signaling is greater than 10, or greater than 100, or greater than 1000, or 10,000 or more.

As used herein, the term “partial agonist” is understood to refer to a compound having agonism to any extent that is lesser than that of a reference standard full agonist. For example, the reference compound for 5-HT_{2A} receptor agonism is alpha-methylserotonin. A compound which has a maximum efficacy (Eₘₐₓ) that is less than 100% of the maximum efficacy for alpha-methylserotonin is a partial agonist.

The term “hallucinogen” refers to a compound which causes hallucinogenic symptoms, which are any one or more symptoms selected from visual hallucinations, auditory hallucinations, visual distortions (such as drifting, morphing, breathing or melting of objects and surfaces in the field of view), detachment from reality, dissociation, delirium, and undesired altered states of consciousness. A compound of the present disclosure is considered “non-hallucinogenic” if at doses which are therapeutically effective for the treatment of neuropsychiatric disorders described herein (e.g., depression, anxiety, etc.) the compound does not cause hallucinogenic symptoms.

“Alkyl” as used herein is a saturated or unsaturated hydrocarbon moiety, e.g., one to twenty-one carbon atoms in length, unless indicated otherwise; any such alkyl may be linear or branched (e.g., n-butyl or tert-butyl), preferably linear, unless otherwise specified. For example, “C₁₋₂₁ alkyl” denotes alkyl having 1 to 21 carbon atoms. In one embodiment, alkyl is optionally substituted with one or more hydroxy or C₁₋₂₂alkoxy (e.g., ethoxy) groups. In another embodiment, alkyl contains 1 to 21 carbon atoms, preferably straight chain and optionally saturated or unsaturated, for example in some embodiments wherein R¹ is an alkyl chain containing 1 to 21 carbon atoms, preferably 6-15 carbon atoms, 16-21 carbon atoms, e.g., so that together with the —C(O)— to which it attaches, e.g., when cleaved from the compound of Formula II, forms the residue of a natural or unnatural, saturated or unsaturated fatty acid.

The words “treatment” and “treating” are to be understood accordingly as embracing prophylaxis and treatment or amelioration of symptoms of disease and/or treatment of the cause of the disease. In particular embodiments, the words “treatment” and “treating” refer to prophylaxis or amelioration of symptoms of the disease.

As used herein, the term “brain” or “brain region” may refer to any structural or functional region of the brain, including, but not limited to, the prefrontal cortex (e.g., medial prefrontal cortex, lateral prefrontal cortex, dorsomedial prefrontal cortex, dorsolateral prefrontal cortex, ventromedial prefrontal cortex, ventral prefrontal cortex), amygdala, hippocampus, frontal cortex, orbitofrontal cortex, insula cortex, fronto-insular cortex, anterior cingulate cortex, subcallosal cingulate cortex, ventral tegmental area, ventral palladium, nucleus accumbens, supramarginal gyrus, inferior temporal gyrus, and the subgenual cingulate area, Each of these brain regions has been associated with one or more of emotion, cognition, depression, psychosis, bipolar disorder, anxiety, and other neuropsychiatric disorders. There is evidence that several neuropsychiatric disorders, including depression and bipolar disorder, may be associated with changes in tissue volume in one or more of these brain regions (indicative of loss of neurons and/or “pruning” of synapses), or changes in the synaptic expression or activity of neurotransmitter receptors or transporters. In some embodiments, the brain regions affected by the methods disclosed herein include subregions of the prefrontal cortex (e.g., the mPFC), the amygdala, and/or the hippocampus.

The prefrontal cortex is the anterior region of the frontal lobe of the cerebral cortex, and it is the primary region of the brain responsible for the orchestration of thoughts and actions. It is critical to executive functions, such as planning, decision-making, short-term memory, personality, social behavior, and some aspects of speech and language. The prefrontal cortex (PFC) has several subregions. In humans, the PFC is generally divided into the ventromedial PFC (vmPFC, including the ventral prefrontal cortex, vPFC, and the medial prefrontal cortex, mPFC) and the lateral prefrontal cortex (LPFC, including the dorsolateral prefrontal cortex, dLPFC, and the ventrolateral prefrontal cortex, vLPFC). The mPFC includes the anterior cingulate cortex, which is important for many higher-level functions. The dlPFC is thought to be particularly important in the pathogenesis of depression. In some lower mammals, including rats and mice, functions carried out by the dlPFC in humans are more closely associated with the mPFC.

In some embodiments of the present disclosure, the methods are directed to providing structural remodeling in any of the regions of the PFC, including for example, the mPFC or the dLPFC.

The term “patient” may include a human or non-human patient.

The Diagnostic and Statistical Manual of Mental Disorders, 5th Edition (“DSM-5”), defines “major depressive disorder” (MDD) as having five or more of a set of symptoms during the same two-week period of time, which symptoms represent a change from the patient's previous functioning. The five symptoms are selected from depressed mood, markedly diminished interest or pleasure in almost all activities, significant weight changes, insomnia or hyposomnia, psychomotor agitation or retardation, fatigue, feelings of worthlessness or excessive guilt, diminished ability to think or indecisiveness, and recurrent thoughts of death or suicidal ideation, wherein each of such symptoms is present nearly every day. At a minimum, MDD diagnosis requires at least depressed mood or loss of interest or pleasure as one of the five symptoms. MDD may consist of one or more “major depressive episodes” which can be spaced many weeks or months apart (more than 2 weeks apart to qualify as separate episodes). The DSM-5 notes that there is a risk of suicidal behavior at all time during a major depressive episode.

By its nature, MDD is an acute disorder in so far as the DSM-5 distinguishes it from “persistent depressive disorder”, in which a patient has many of the same symptoms as for MDD, but which persists for at least a 2-year period. In addition to MDD, the DSM-5 also defines a “short-duration depressive episode” as having a depressed affect and at least four of the other symptoms which define MDD for at least 4 days, but less than 14 days. The DSM further defines “recurrent brief depression” as the concurrent presence of depressed mood and at least four other symptoms of depression for 2 to 13 days at least once per month, and persisting for at least 12 consecutive months. Thus, recurrent brief depression similarly consists of brief episodes of depression which recur regularly.

The DSM-5 also includes major depressive episodes as one of the diagnostic criteria for a patient suffering from bipolar disorder. Thus, a patient presenting a major depressive episode may be suffering from either major depressive disorder or bipolar disorder.

It is apparent that there are is a particular need for effective treatment of depression during the earliest stages of a major depressive episode, since each day of such episode can have profound consequences for a patient, yet typical SSRI anti-depressive agents take up to 2-4 weeks for beneficial effects to appear. The same is true for treatment of short duration depressive episodes as well as individual episodes of recurrent brief depression.

The DSM-5 categorizes what has traditionally been termed “post-partum depression” or “peri-partum depression” as a merely a sub-type of the DSM's recognized depressive disorders, rather than as an independent depressive disorder. Thus, both major depressive disorder and acute depressive disorders can be diagnosed as being “with peripartum onset” (DSM-5 also does not distinguish peri-versus post-partum). Thus, as used herein, any of the depression indications may be considered to include such depression indication with peri-partum or post-partum onset, and thus, these indications embrace post-partum and peri-partum depression as well.

The DSM-5 defines a variety of anxiety disorders, including generalized anxiety disorder, panic disorder, social anxiety disorder, and specific phobias. Like the depressive disorders discussed above, anxiety disorders can be marked by recurrent episodes of short duration, such as panic attacks, which may persist over the course of a chronic disorder. For example, generalized anxiety disorder is defined by the DSM-5 to require excessive anxiety and worry occurring more days that not for at least 6 months, about a number of events or activities. A panic attack is defined as an abrupt surge of intense fear or intense discomfort that reaches a peak within minutes, but it can repeatedly recur in response to either expected stimuli or unexpected stimuli. Thus, as for the depressive disorders described above, there is a need for rapidly-acting anxiolytic agents that can treat the symptoms of anxiety or panic, yet some of the most common treatments for anxiety disorders are the SSRIs and other antidepressant agents which take 2-4 weeks to provide relief.

Social avoidance can be a critical and debilitating symptom in patients suffering from anxiety disorders, especially social anxiety disorder, as well as in patients suffering from traumatic anxiety disorders. Social avoidance is often one of the key determinants of whether a person with a severe anxiety disorder is capable of maintaining familial relationships or employment relationships. It has been unexpectedly found that Compounds of the Disclosure are effective in treating the emotional experience symptoms of psychiatric disorders (e.g., the emotional experience negative symptoms of schizophrenics). Negative symptoms of schizophrenia can be divided into two categories: emotional experience (e.g., emotional withdrawal, passive social withdrawal, active social avoidance) and emotional expression (e.g., blunted effect, poor rapport, lack of spontaneity, and motor retardation). In two clinical studies of patients with acute exacerbated schizophrenia, administration of lumateperone once daily (60 mg P.O.), for up to 28 days, resulted in a significant and unexpected improvement in symptoms of emotional experience compared to placebo. These are the symptoms that are most highly correlated with interpersonal functioning. As such, Compounds of the Disclosure may be highly effective in treating the emotional experience symptoms of other psychiatric disorders, such as social anxiety disorders, or any other psychiatric disorders in which social withdrawal and social avoidance are symptoms.

If not otherwise specified or clear from context, the following terms herein have the following meanings:

The Compounds of the Disclosure, as described herein, may be in free or pharmaceutically acceptable salt form. Pharmaceutically acceptable salts include, for example, the tosylate salts in the case of Compounds of Formula I or II. Where dosages or amounts of a salt are given by weight, e.g., milligrams per day or milligrams per unit dose, the dosage amount of the salt is given as the weight of the corresponding free base, unless otherwise indicated.

The term “concurrently” when referring to a therapeutic use means administration of two or more active ingredients to a patient as part of a regimen for the treatment of a disease or disorder, whether the two or more active agents are given at the same or different times or whether given by the same or different routes of administrations. Concurrent administration of the two or more active ingredients may be at different times on the same day, or on different dates or at different frequencies.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

With respect to concurrent treatment using Compounds of the Disclosure and an NMDA receptor antagonist (e.g., ketamine), without being bound by theory, it is believed that the combination of these agents would permit lower doses of both agents to be provide the desired effects according to the methods described herein, such that the dissociative effects produced by the NMDA receptor antagonist would be minimized while the synergistic effects would be maximized.

Dosages employed in practicing the present disclosure will of course vary depending, e.g., on the particular disease or condition to be treated, the particular active compounds used, the mode of administration, and the therapy desired. Unless otherwise indicated, an amount of an active compound for administration (whether administered as a free base or as a salt form) refers to or is based on the amount of the compound in free form (i.e., the calculation of the amount is based on the amount of active moiety in free form, not taking into account the weight of the counter ion in the case of a salt). The Compounds of the Disclosure may be administered by any suitable route, including oral, parenteral, transdermal, or transmucosal, for example in the form of a tablet, a capsule, a subcutaneous injection, or an oral, rapidly disintegrating tablet or film for sublingual or buccal administration.

For the avoidance of doubt, any disclosure of a numerical range, e.g., “up to X” amount is intended to include the upper numerical limit X. Therefore, a disclosure of “up to 60 mg” is intended to include 60 mg.

Pharmaceutical compositions comprising compounds of the Disclosure may be prepared using conventional diluents or excipients and techniques known in the galenic art. Thus, oral dosage forms may include tablets, capsules, solutions, suspensions and the like.

Compounds of the Disclosure may be included as a depot formulation, e.g., by dispersing, dissolving, suspending, or encapsulating the Compounds of the Disclosure in a polymeric matrix as described in herein, such that the Compound is continually released as the polymer degrades over time. The release of the Compounds of the Disclosure from the polymeric matrix provides for the controlled- and/or delayed- and/or sustained-release of the Compounds, e.g., from the pharmaceutical depot composition, into a subject, for example a warm-blooded animal such as man, to which the pharmaceutical depot is administered. Thus, the pharmaceutical depot delivers the Compounds of the Disclosure to the subject at concentrations effective for treatment of the particular disease or medical condition over a sustained period of time, e.g., 1 week to 3 months.

Polymers useful for the polymeric matrix in the Composition of the Disclosure (e.g., Depot composition of the Disclosure) may include a polyester of a hydroxyfatty acid and derivatives thereof or other agents such as polylactic acid, polyglycolic acid, polycitric acid, polymalic acid, poly-beta.-hydroxybutyric acid, epsilon.-capro-lactone ring opening polymer, lactic acid-glycolic acid copolymer, 2-hydroxybutyric acid-glycolic acid copolymer, polylactic acid-polyethyleneglycol copolymer or polyglycolic acid-polyethyleneglycol copolymer), a polymer of an alkyl alpha-cyanoacrylate (for example poly(butyl 2-cyanoacrylate)), a polyalkylene oxalate (for example polytrimethylene oxalate or polytetramethylene oxalate), a polyortho ester, a polycarbonate (for example polyethylene carbonate or polyethylenepropylene carbonate), a polyortho-carbonate, a polyamino acid (for example poly-gamma.-L-alanine, poly-.gamma.-benzyl-L-glutamic acid or poly-y-methyl-L-glutamic acid), a hyaluronic acid ester, and the like, and one or more of these polymers can be used.

If the polymers are copolymers, they may be any of random, block and/or graft copolymers. When the above alpha-hydroxycarboxylic acids, hydroxydicarboxylic acids and hydroxytricarboxylic acids have optical activity in their molecules, any one of D-isomers, L-isomers and/or DL-isomers may be used. Among others, alpha-hydroxycarboxylic acid polymer (preferably lactic acid-glycolic acid polymer), its ester, poly-alpha-cyanoacrylic acid esters, etc. may be used, and lactic acid-glycolic acid copolymer (also referred to as poly(lactide-alpha-glycolide) or poly(lactic-co-glycolic acid), and hereinafter referred to as PLGA) are preferred. Thus, in one aspect the polymer useful for the polymeric matrix is PLGA. As used herein, the term PLGA includes polymers of lactic acid (also referred to as polylactide, poly(lactic acid), or PLA). Most preferably, the polymer is the biodegradable poly(d,l-lactide-co-glycolide) polymer, such as PLGA 50:50, PLGA 85:15 and PLGA 90:10.

In a preferred embodiment, the polymeric matrix is a biocompatible and biodegradable polymeric material. The term “biocompatible” is defined as a polymeric material that is not toxic, is not carcinogenic, and does not significantly induce inflammation in body tissues. The matrix material should be biodegradable wherein the polymeric material should degrade by bodily processes to products readily disposable by the body and should not accumulate in the body. The products of the biodegradation should also be biocompatible with the body in that the polymeric matrix is biocompatible with the body. Particular useful examples of polymeric matrix materials include poly(glycolic acid), poly-D,L-lactic acid, poly-L-lactic acid, copolymers of the foregoing, poly(aliphatic carboxylic acids), copolyoxalates, polycaprolactone, polydioxanone, poly(ortho carbonates), poly(acetals), poly(lactic acid-caprolactone), polyorthoesters, poly(glycolic acid-caprolactone), polyanhydrides, and natural polymers including albumin, casein, and waxes, such as, glycerol mono- and distearate, and the like. The preferred polymer for use in the practice of this invention is dl(polylactide-co-glycolide). It is preferred that the molar ratio of lactide to glycolide in such a copolymer be in the range of from about 75:25 to 50:50.

Useful PLGA polymers may have a weight-average molecular weight of from about 5,000 to 500,000 Daltons, preferably about 150,000 Daltons. Dependent on the rate of degradation to be achieved, different molecular weight of polymers may be used. For a diffusional mechanism of drug release, the polymer should remain intact until all of the drug is released from the polymeric matrix and then degrade. The drug can also be released from the polymeric matrix as the polymeric excipient bioerodes.

The PLGA may be prepared by any conventional method, or may be commercially available. For example, PLGA can be produced by ring-opening polymerization with a suitable catalyst from cyclic lactide, glycolide, etc. (see EP-0058481B2; Effects of polymerization variables on PLGA properties: molecular weight, composition and chain structure).

It is believed that PLGA is biodegradable by means of the degradation of the entire solid polymer composition, due to the break-down of hydrolysable and enzymatically cleavable ester linkages under biological conditions (for example in the presence of water and biological enzymes found in tissues of warm-blooded animals such as humans) to form lactic acid and glycolic acid. Both lactic acid and glycolic acid are water-soluble, non-toxic products of normal metabolism, which may further biodegrade to form carbon dioxide and water. In other words, PLGA is believed to degrade by means of hydrolysis of its ester groups in the presence of water, for example in the body of a warm-blooded animal such as man, to produce lactic acid and glycolic acid and create the acidic microclimate. Lactic and glycolic acid are by-products of various metabolic pathways in the body of a warm-blooded animal such as man under normal physiological conditions and therefore are well tolerated and produce minimal systemic toxicity.

Compounds of the Disclosure, and methods for their synthesis, including the synthesis of intermediates, have been disclosed in, for example, Li, et al., Journal of Medicinal Chemistry 57:2670-2682 (2014), U.S. Pat. Nos. 6,713,471, 6,552,017, 7,071,186, 8,309,722, U.S., 9,708,322, U.S. Pat. Nos. 10,245,260, 10,688,097, 10,961,245, 10,906,906, 11,427,587, 11,453,670, US 2022/0048910, US 2022/0041600, and US 2022/0064166, and U.S. Provisional Application No. 63/478,010, the contents of which are hereby incorporated by reference in their entireties.

The synthesis of similar fused gamma-carbolines has been disclosed in, for example, U.S. Pat. Nos. 8,309,722, 8,993,572, US 2017/0183350, WO 2018/126140 and WO 2018/126143, the contents of each of which are incorporated by reference in their entireties. Compounds of the present disclosure can be prepared using similar procedures.

Salts of Compounds of the Disclosure may be prepared as similarly described in U.S. Pat. Nos. 6,548,493; 7,238,690; 6,552,017; 6,713,471; 7,183,282, 8,648,077; 9,199,995; 9,586,860; U.S. RE39680; and U.S. RE39679, the contents of each of which are incorporated by reference in their entirety.

The Compounds of Formula II, described herein, have been disclosed, and their synthetic methods provided, in International Application No. PCT/US2023/86562, which is hereby incorporated by reference herein in its entirety.

### EXAMPLES

### Example 1. 1-(4-fluorophenyl)-3-((6bR,10aS)-3-methyl-2,3,6b,7,10,10a-hexahydro-1H-pyrido[3′,4′: 4,5]pyrrolo[1,2,3-de]quinoxalin-8 (9H)-yl) propan-1-one

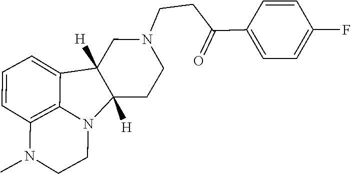 [SMILES: CN1CCN2c3c(cccc31)[C@@H]1CN(CCC(=O)c3ccc(F)cc3)CC[C@@H]12]

To a degassed solution of (6bR,10aS)-3-methyl-2,3,6b,7,8,9,10,10a-octahydro-1H-pyrido[3′,4′: 4,5]pyrrolo[1,2,3-de]quinoxaline hydrochloride (3.0 g, 11.3 mmol) in anhydrous dioxane (20 mL) is added N, N-diisopropylethylamine (3.0 g, 22.6 mmol), 3-chloro-1-(4-fluorophenyl) propan-1-one (2.3 g, 12.4 mmol), potassium iodide (2.3 g, 13.6 mmol) and a catalytic amount of 18-crown-6 under argon. The resulting mixture is heated to 95° C. and stirred for 6.5 hours. After cooling to room temperature, the solvent is removed, and the residue is suspended in ethyl acetate (50 mL) and water (50 mL). The aqueous phase is separated and extracted twice with ethyl acetate (30 mL). The combined organic phase is dried over MgSO₄ and concentrated. The residue is purified by silica gel column chromatography using a gradient of 0-20% mixed solvents [ethyl acetate/methanol/7N NH₃ in methanol (10:1:0.1 v/v)] in ethyl acetate to obtain the title product as a brown solid (0.8 g, yield 16%). MS (ESI) m/z 380.2 [M+1]⁺. ¹H NMR (500 MHz, DMSO) δ 9.15 (s, 1H), 8.19-8.07 (m, 2H), 7.42 (t, J=8.8 Hz, 2H), 6.62 (t, J=7.7 Hz, 1H), 6.50 (d, J=7.3 Hz, 1H), 6.44 (d, J=7.9 Hz, 1H), 3.68-3.57 (m, 3H), 3.53-3.41 (m, 5H), 3.35 (q, J=2.6 Hz, 1H), 3.23 (d, J=5.8 Hz, 1H), 3.14 (q, J=13.1 Hz, 1H), 2.82 (s, 4H), 2.76-2.61 (m, 2H), 2.29 (d, J=15.5 Hz, 1H), 2.07 (t, J=14.8 Hz, 1H).

### Example 2: Evaluation of a Compound of Formula I and/or Compound of Formula II for Effects on Dendritic Spine Turnover in Mice

One or more Compounds of Formula I or II (the “Compound” or “Compounds”) are evaluated using procedures as more fully described in Shao et al., Neuron, 109 (16): 2535-2544 (2021), such as the Compound of Example 1. The purpose of the study is to evaluate dendritic spine turnover (formation of new spines and loss of spines) in mice as a function of treatment with the Compound(s). The study analyzes the effects of acute and chronic Compound administration on longitudinal spine analyses in the mPFC using two-photon imaging technique (see below) in anesthetized mice. Because Shao et al. found differential effects of the psilocybin in males versus females, the effects of the Compound(s) on spine formation in female mice will also be measured.

Briefly, male and female transgenic mice Thy 1^{GFP} (line M) mice are obtained from Jackson Laboratory and are 4 to 8-weeks old at receipt in order to be used for imaging about 2 weeks later. Mice are group housed (2-5 mice per cage) under controlled temperature in a 12-hour light-dark cycle with free access to food and water.

The Compound(s) are formulated about 30 minutes before use in a vehicle composed of 5% DMSO, 5% Tween-20, 15% PEG-400 and 75% water for the acute injection. Compound and vehicle are administered by intraperitoneal route (i.p.) or subcutaneous route (s.c.). Psilocybin is administered in a saline vehicle by either i.p. or s.c. route.

The mice are divided into at least four groups: (1) no stress, vehicle treatment; (2) stress, vehicle treatment; (3) stress, Compound treatment (10 mg/kg, i.p. or s.c.); (4) stress, psilocybin (1 mg/kg, i.p.) treatment. There are 4-6 animals per group. If more than one Compound is tested, then additional groups (3) may be employed. In addition, cither one of more of the Compounds and the psilocybin may be evaluated both after acute treatment (single injection) or chronic treatment (daily injection for a period of days or a single long-acting injection), and thus, additional groups may be employed.

Drug injections (vehicle, Compound(s), or psilocybin) are given once daily beginning on either day −15, day −7, or day 0. Alternatively, a single drug injection (vehicle, Compound, or psilocybin) is given once on either day −15, day −7, or day 0. Imaging is conducted through day 22, for example, at days −15, −3, −1, 1, 8, 15, and 22 (+/−1 day).

Restraint stress is a model of chronic stress-induced depression that has been described to lead to robust morphological and neurochemical brain alterations, as well as behavioral and cognitive deficits Stress is applied as chronic restraint stress from day −21 to day −1 or day 0, according to standard procedures (e.g., Buynitsky et al. 2009, PMID: 19463853; Chiba et al. 2012, PMID: 22664354; Jaggi et al. 2011, PMID: 21927881; O'Mahony et al., 2011, PMID: 21110995; Ju et al. 2022, PMID: 35291971; Codeluppi et al., 2021. PMID: 34346493). Briefly, restraint stress is performed using either one of two methods. In one method, mice are individually placed head-first into a well-ventilated 50 ml Falcon polypropylene conical tube with a small hole at each extremity of the tube (bottom and cap). The nose of the mouse is the closest to the bottom hole, hence assuring proper ventilation. Mice are not able to move forward or backward in this device and are maintained in the restraint tube placed on a secure surface at room temperature under the hood of the Biological Safety Cabinet. Alternatively, restraint stress maybe performed in a mouse restrainer device (e.g., Stoelting Ref #51338 Cylindrical Restrainer or a tapered plastic DecapiCone). Pilot experiments are performed to test compatibility of these two alternatives with the imaging head plate. After the restraint period, the mice are returned to their home cages in the animal facility and allowed free access to food and water until the next restraint cycle.

Spine density is assessed from baseline before stress on day −21 to day 22. Spine formation and elimination rates are assessed during stress on day −3 and day −1, and after treatment on day 1.

Prior to surgery, each mouse is injected with carprofen (5 mg/kg, s.c.) and dexamethasone (3 mg/kg, i.m.). During surgery, each mouse is anesthetized with isoflurane (3-4% for induction and 1-1.5% for the remainder of surgery) and fixed in a stereotaxic apparatus (David Kopf Instruments). The body of the mouse rests on a water-circulating heating pad set to 38° C. Prior to the procedure, the hair on the head is shaved, and the scalp is then wiped and disinfected with ethanol pad and betadine. An incision is made to remove the skin and the connective tissue above the skull is removed. Subsequently, a dental drill is used to make an about 3-mm-diameter circular craniotomy above the right medial frontal cortex (center position: +1.5 mm anterior-posterior, AP; +0.4 mm medial-lateral, ML; relative to bregma). Artificial cerebrospinal fluid (ACSF, containing (in mM): 135 NaCl, 5 HEPES, 5 KCl, 1.8 CaCl₂, 1 MgCl₂; pH 7.3) is used to irrigate the exposed dura above brain. A two-layer glass window is made from two round 3-mm-diameter, #1 thickness glass coverslip, bonded by UV-curing optical adhesive. The glass window is carefully placed over the craniotomy and, while maintaining a slight pressure, adhesive (Henkel Loctite 454) is used to secure the glass window to the surrounding skull. A stainless steel headplate is affixed on the skull with C&B Metabond (Parkell) centered on the glass window. Carprofen (5 mg/kg, s.c.) is given to the mouse immediately after surgery and on each of the following 3 days. The mouse recovers for at least 20 days after the surgery and before the start of imaging experiments.

A two-photon microscope (Movable Objective Microscope, Sutter Instrument) is controlled by ScanImage 2020 software21. The laser excitation is provided by a tunable Ti: Sapphire femtosecond laser (Chameleon Ultra II, Coherent) and focused onto the mouse brain with a water-immersion 20× objective (XLUMPLFLN, 20×/0.95 N.A., Olympus). The laser power measured at the objective is less than or equal to 40 mW. During an imaging session, the mouse is head fixed and anesthetized with 1-1.5% isoflurane. Body temperature is controlled using a heating pad and a DC Temperature Controller with rectal thermistor probe feedback. Each imaging session does not exceed 2 hours. Apical tuft dendrites are imaged at 0-200 μm below the dura. Multiple fields of view are imaged in the same mouse. For each field of view, 10 to 40-μm-thick image stacks are collected at 1 μm steps and at 1024× 1024 pixels at 0.11 μm per pixel resolution.

For longitudinal imaging, the same fields of view are returned to across imaging sessions by locating and triangulating from a landmark on the left edge of the glass window. Control mice receive an equal volume s.c. injection of the pre-made vehicle solution.

Structural parameters such as spine head width and spine protrusion length are quantified based on a standardized protocol. Briefly, if a protrusion extends for more than 0.4 μm from the dendritic shaft, a dendritic spine is counted. The head width of a dendritic spine is measured as the width at the widest part of the head of the spine. The protrusion length of a dendritic spine refers to the distance from its root at the shaft to the tip of the head. The line segment tool in ImageJ will be used to measure the distances. Changes in spine density, spine head width, and spine protrusion length, across imaging sessions are shown as fold-change from the value measured on the first imaging session (day −3) for each dendritic segment. The spine formation rate is calculated as the number of dendritic spines newly formed between two consecutive imaging sessions divided by the total number of dendritic spines observed in the first imaging session. The spine elimination rate is calculated as the number of dendritic spines lost between two consecutive imaging sessions divided by the total number of dendritic spines observed in the first imaging session. To quantify the persistence of newly formed spines, the number of dendritic spines newly formed on day 1 that are still be present on day 15 and 22 is calculated, and divided by the total number of newly formed dendritic spines on day 1.

These results of the study will show that the test Compounds, and psilocybin, both promote the formation of new dendritic spines, and otherwise promote dendritic growth and neuritogenesis.

### Example 3: Evaluation of a Compound of Formula I and/or Compound of Formula II for Effects on mTOR Signaling in the Brain Pre-Frontal Cortex (PFC)

Male adult mice are injected SC with either test compound (1 mg/kg and/or 3 mg/kg and/or 10 mg/kg) or vehicle. At 24 hours post-injection, samples from the brain (e.g., the pre-frontal cortex (PFC) region or the amygdala) are collected, and a synaptoneurosome-enriched fraction is collected and prepared for Western blotting. Quantitative analysis of phospho (p) protein immunoblots are determined relative to total levels of each protein. Changes in the amount of phosphorylated ERK, Akt, mTOR, and P70S6K proteins, in the tested brain regions are determined relative to vehicle-treated mice, as previously described (Dutheil, et al., J. Neuroscience, 43 (5): 863-77, 2023).

The test compounds are found to stimulate mTOR signaling in a dose-dependent fashion in the mouse medial PFC, as demonstrated by increases in one or more of p-ERK, p-mTOR, and p-P70s6k in the tested brain regions. The mTOR signaling pathway has been shown to contribute to neuroplasticity and enhanced cognitive function and it is altered in brain regions associated with major depressive disorder. Rapid-acting antidepressants have been reported to stimulate this pathway in the prefrontal cortex. Similar results are obtained using samples from the amygdala. Further studies are performed using hippocampus brain samples.

These results will support that the compound of the present disclosure, through their effects on the mTOR signaling pathway, are expected to provide enhanced neural growth, enhanced neuritogenesis, enhanced neurite outgrowth, enhanced neural connectivity, increased synaptic density, increased dendritic spine density, increased dendritic spine size, and/or increasing excitatory neurotransmission, and other effects described herein, in the brain of a human or animal or in neural cell or neural tissue, in vivo or in vitro.

In further work, the compounds of the present disclosure may be studied using in vivo or in vitro examination of the synaptic intensity of fluorescent-tagged neurotransmitter receptors or transporters, for example GFP (green fluorescent protein)- or SEP (super-ecliptic pHluorin)-tagged receptors or transporters, such as AMPA-type glutamate receptors. Various methods of fluorescence microscopy can be utilized, optionally with machine-learning-enhanced analytical methods, such as those described in Xu et al., “Cross-modality supervised image restoration enables nanoscale tracking of synaptic plasticity in living mice,” Nature Methods 20:935-944 (June 2023; published online May 2023). Such methods may be applied to the analysis of the function, activity, and distribution of numerous synaptic receptors and transporters, such as serotonin receptor (e.g., 5-HT_{2A}, or 5-HT_{2C}), dopamine receptor (e.g., D1 or D2), norepinephrine receptor, glutamate receptor (e.g., NMDA-type, AMPA-type, or mGluR-type), GABA receptor (e.g., GABAA or GABAB), serotonin transporter (SERT), dopamine transporter (DAT), norepinephrine transporter (NET), glutamate transporter (e.g., EAAT or VGLUT), and/or GABA transporter (e.g., GAT1, GAT3) density in the synapses.

The Examples provided herein are exemplary only and are not intended to be limiting in any way to the various aspects and embodiments of the invention described herein.

1. A method for enhancing neural growth, enhancing neural connectivity, increasing synaptic density, increasing dendritic spine density, increasing dendritic spine size, and/or increasing excitatory neurotransmission, or enhancing neuritogenesis and/or neurite outgrowth in the brain of a subject in need thereof, e.g., in the prefrontal cortex region of the brain, the method comprising the step of administering an effective amount of a Compound of Formula I, 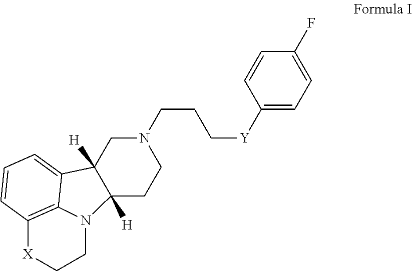
wherein X is selected from —O—, —S—, —N(H), and —N(CH₃)—, and Y is selected from —O—, —C(O)—, —CH(OH)—, and —CH(OCH₃);
or a deuterated analog thereof, in free, or pharmaceutically acceptable salt form;
or
an effective amount of a Compound of Formula II: 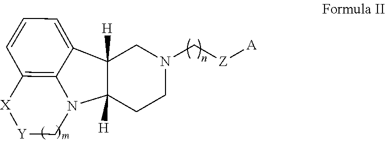
wherein:
X is selected from S, S(O), S(O)₂, O, CH₂, CHR^{b}, C(R^{b})₂, NH, N(R^{a}) (e.g., N(CH₃)), N—C(O)—R^{a}, N—C(O)—O—R^{a}, N—C(O)—O—CH₂—O—R^{a}, N—CH₂—O—C(O)—R^{a}, N⁺(═O⁻), a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Y is CH₂, CHR^{c}, —C(O)—, C(R^{c})₂, a spiro-joined C₃₋₆cycloalkyl (e.g., cyclopropane), or a spiro-joined 3-6-membered heterocycloalkyl (e.g., aziridine or oxetane), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
Z is a bond, —S—, S(O), S(O)₂, —O—, —NH, N(R^{d}), —C(O)—, —C(OH)—, —C(OC₁₋₆alkyl), —C(═N—OH)—, —C(═N—OC₁₋₆alkyl)-, a spiro-joined C₃₋₆cycloalkyl (e.g., cyclopropane), a spiro-joined 3-6-membered heterocycloalkyl (e.g., aziridine or oxetane), or —O(CH₂)ₚO— wherein p is 2, 3, or 4 (e.g., p is 2), wherein said spiro-joined C₃₋₆cycloalkyl or 3-6-membered heterocycloalkyl is optionally substituted by one or more groups selected from C₁₋₆alkyl (e.g., methyl), haloC₁₋₆alkyl (e.g., trifluoromethyl), C₁₋₆alkoxy (e.g., methoxy), C₃₋₆cycloalkyl (e.g., cyclopropyl), C₃₋₆cycloalkoxy (e.g., cyclopropoxy), and hydroxy;
A is H, C₃₋₆cycloalkyl (e.g., cyclopropyl or cyclohexyl), aryl (e.g., phenyl), or heteroaryl, wherein said cycloalkyl, aryl, or heteroaryl is substituted by 0-5 groups R;
each R is independently selected from aryl (e.g., phenyl), aryloxy (e.g., phenoxy), heteroaryl (e.g., pyridyl), C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy, ethoxy), C₁₋₆alkylthio (e.g., methylthio), halo (e.g., F), cyano, C₃₋₆cycloalkyl (e.g., cyclopropyl), C₃₋₆cycloalkoxy (e.g., cyclopropoxy), or hydroxy, wherein each of said aryl, heteroaryl, alkyl, haloalkyl, alkylsulfonyl, alkoxy, alkylthio, cycloalkyl, or cycloalkoxy, is optionally further substituted by one or more groups selected from aryl (optionally substituted with halo), halo, C₁₋₆alkyl (e.g., methyl), haloC₁₋₆alkyl (e.g., trifluoromethyl), C₁₋₆alkylsulfonyl (e.g., methylsulfonyl), C₁₋₆alkoxy (e.g., methoxy), C₁₋₆alkylthio (e.g., methylthio), C₃₋₆cycloalkyl (e.g., cyclopropyl), C₃₋₆cycloalkoxy (e.g., cyclopropoxy), amino, C₁₋₆alkylamino (e.g., methylamino), di(C₁₋₆alkyl)amino (e.g., dimethylamino), (C₁₋₆alkyl) (C₁₋₆alkyl)amino (e.g., methylethylamino), and hydroxy;
R^{a} and R^{d}, are each independently selected from C₁₋₂₀alkyl (e.g., methyl or tert-butyl), and C₁₋₂alkylaryl (e.g., benzyl or phenethyl);
R^{b} and R^{e} are each independently selected from C₁₋₆alkyl (e.g., methyl, ethyl, tert-butyl), C₁₋₆alkoxy, C₃₋₆cycloalkyl (e.g., cyclopropyl), C₃₋₆cycloalkoxy (e.g., cyclopropoxy), and C₁₋₂alkylaryl (e.g., benzyl or phenethyl);
m is 1 or 2; and
n is 1, 2, 3, 4, or 5;
in free or pharmaceutically acceptable salt form;
provided that n is not 3 when Z is —C(O)—, X is CH₂ or O, and m is 2; and
provided that n is not 3 when Z is —C(O)—, X is CH₂, and m is 1; and
provided that n is not 3 when Z is —C(O)— or —O—, X is NH or N(R^{a}), and m is 1;
and provided that n is not 3 when Z is O, X is NCH₃, Y is —C(O)—, and m is 1; to the subject.

2. The method according to claim 1, wherein the Compound of Formula II is: 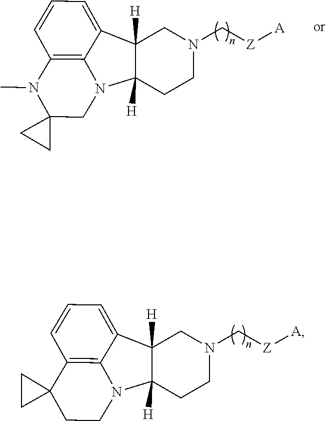
wherein n, Z, and A are defined as in claim 1,
in free, or pharmaceutically acceptable salt form.

3. The method according to claim 1, wherein the Compound of Formula I or deuterated analog thereof is non-deuterated lumateperone, i.e., having the following structure: 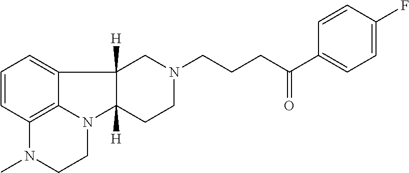 [SMILES: CN1CCN2c3c(cccc31)[C@@H]1CN(CCCC(=O)c3ccc(F)cc3)CC[C@@H]12]
in free or pharmaceutically acceptable salt form.

4. The method according to claim 1, wherein the Compound of Formula II is: 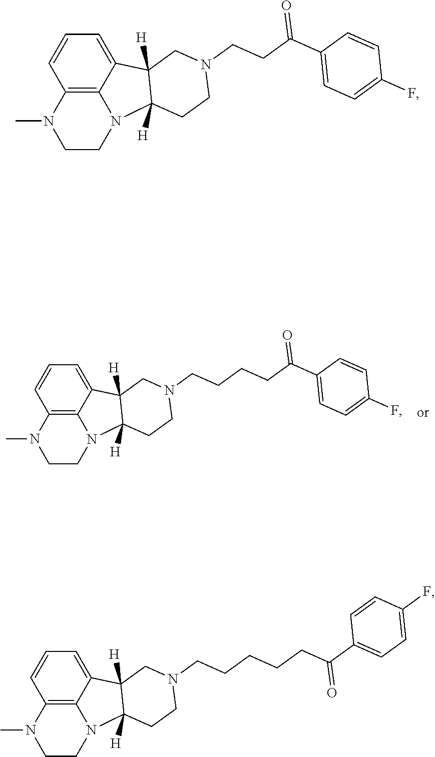 [SMILES: CN1CCN2c3c(cccc31)[C@@H]1CN(CCC(=O)c3ccc(F)cc3)CC[C@@H]12 ; CN1CCN2c3c(cccc31)[C@@H]1CN(CCCCC(=O)c3ccc(F)cc3)CC[C@@H]12 ; CN1CCN2c3c(cccc31)[C@@H]1CN(CCCCCC(=O)c3ccc(F)cc3)CC[C@@H]12]
in free or pharmaceutically acceptable salt form.

5. The method according to claim 1, wherein the Compound of Formula I or deuterated analog thereof, or the Compound of Formula II, is in the form of a pharmaceutically acceptable salt.

6. The method according to claim 5, wherein the pharmaceutically acceptable salt is a toluenesulfonic acid addition salt (e.g., a mono-tosylate salt or a bis-tosylate salt).

7. The method according to claim 1, wherein the method provides enhanced neural growth in the brain of the subject (e.g., in the prefrontal cortex region of the brain).

8. The method according to claim 1, wherein the method provides enhanced neural connectivity, in the brain of the subject (e.g., in the prefrontal cortex region of the brain).

9. The method according to claim 1, wherein the method provides increased synaptic density, in the brain of the subject (e.g., in the prefrontal cortex region of the brain).

10. The method according to claim 1, wherein the method provides increased dendritic spine density, in the brain of the subject (e.g., in the prefrontal cortex region of the brain).

11. The method according to claim 1, wherein the method provides increased dendritic spine size (e.g., increased width of spine heads and/or increased spine protrusion lengths), in the brain of the subject (e.g., in the prefrontal cortex region of the brain).

12. The method according to claim 1, wherein the method provides increased excitatory neurotransmission (e.g., enhanced glutamatergic transmission or increased rate of mEPSCs), in the brain of the subject (e.g., in the prefrontal cortex region of the brain).

13. The method according to claim 7, wherein the enhanced neural growth, enhanced neural connectivity, increased synaptic density, increased dendritic spine density, increased dendritic spine size, and/or increased excitatory neurotransmission is characterized by or caused by enhanced neuritogenesis and/or enhanced neurite outgrowth.

14. The method according to claim 1, wherein the method enhances neuritogenesis and/or enhances neurite outgrowth.

15. The method according to claim 1, wherein the method provides the enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain) within less than 4 weeks of the initiation of administration of the compound, e.g., less than 3 weeks, less than 2 weeks, less than 1 week, less than 6 days, less than 5 days, less than 4 days, less than 3 days, or less than 2 days, after the initiation of treatment with the compound.

16. The method according to claim 1, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after the cessation of administration of the compound, e.g., for at least 3 weeks, or at least 4 weeks, or at least 2 months, or at least 3 months.

17. The method according to claim 1, wherein the method maintains at least 50% of the peak enhanced neuritogenesis, enhanced neurite outgrowth, enhanced neural growth, enhanced neural connectivity, increased synaptic density, increased dendritic spine density, increased dendritic spine size, and/or increased excitatory neurotransmission, in the brain of the subject (e.g., in the prefrontal cortex region of the brain), for at least 2 weeks after administration of a single dose of the compound, e.g., for at least 3 weeks, or at least 4 weeks, or at least 2 months, or at least 3 months.

18. The method according to claim 1, wherein the method does not cause hallucinogenic side effects.

19. A method for enhancing neural growth, enhancing neuritogenesis, enhancing neurite outgrowth, enhancing neural connectivity, increasing synaptic density, increasing dendritic spine density, increasing dendritic spine size, and/or increasing excitatory neurotransmission, in a neural cell or neural tissue (e.g., a cell culture), in vitro or in vivo, the method comprising the step of contacting the neural cell or neural tissue with an effective amount of a Compound of Formula I or a deuterated analog thereof, or a Compound of Formula II, each in free or pharmaceutically acceptable salt form, as described in claim 1.

20. The method according to claim 1, wherein the Compound of Formula II is: 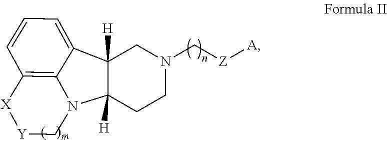
wherein the variables are defined as provided in any of the following embodiments:

| X | Y | m | n | Z | A |
|---|---|---|---|---|---|
| —N(CH₃)— | —CH₂— | 1 | 3 | —O— | 2-CN-4-F-phenyl |
| —N(CH₃)— | —CH₂— | 1 | 4 | —C(O)— | 4-F-phenyl |
| —N(CH₃)— | —CH₂— | 1 | 2 | —C(O)— | 4-F-phenyl |
| —N(CH₃)— | Cyp | 1 | 3 | —C(O)— | 4-F-phenyl |
| —N(CH₃)— | —CH₂— | 1 | 5 | —C(O)— | 4-F-phenyl |
| —N(CH₃)— | —CH₂— | 1 | 3 | —C(O)— | 4-(CH₃OCH₂CH₂O)-phenyl |
| Cyp | —CH₂— | 1 | 3 | —C(O)— | 4-F-phenyl |
| Cyp | —CH₂— | 1 | 3 | —O— | 4-F-phenyl |
| —NH— | —C(O)— | 1 | 3 | —O— | 3-Me-4-F-phenyl |
| —NH— | —C(O)— | 1 | 3 | —O— | 3-Cl-4-F-phenyl |
| —NH— | —C(O)— | 1 | 3 | —O— | 3-CN-4-F-phenyl |
| —N(CH₃)— | —CH₂— | 1 | 2 | —O(CH₂)₂O— | 4-F-phenyl |
| —N(CH₃)— | —CH₂— | 1 | 3 | —C(O)— | 4-((4-F-benzyl)oxy) |
| —N(CH₃)— | Cyp | 1 | 2 | —C(O)— | 4-F-phenyl |
| —N(CH₃)— | Cyp | 1 | 2 | —O— | 4-F-phenyl |
| —N(CH₃)— | Cyp | 1 | 2 | —C(O)— | 2-Me-4-F-phenyl |
| —N(CH₃)— | Cyp | 1 | 2 | —O— | 2-Me-4-F-phenyl |
| —NH— | —C(O)— | 1 | 2 | —O— | 4-F-phenyl |
| —N(CH₃)— | Cyp | 1 | 3 | bond | 3-Cl-phenyl |
| —N(CH₃)— | Cyp | 1 | 3 | bond | 3-MeO-phenyl |
| —N(CH₃)— | Cyp | 1 | 3 | bond | 3-CF₃-phenyl |
| —N(CH₃)— | Cyp | 1 | 3 | bond | 2-Cl-phenyl |
| —N(CH₃)— | Cyp | 1 | 3 | bond | 2-Me-phenyl |
| —N(CH₃)— | Cyp | 1 | 2 | bond | 2-Cl-phenyl |
| —N(CH₃)— | Cyp | 1 | 2 | bond | 2-MeO-phenyl |
| —N(CH₃)— | —CH₂— | 1 | 2 | —O— | 4-F-phenyl |
| —N(CH₃)— | —CH₂— | 1 | 2 | —O— | 2-MeO-4-F-phenyl |
| —N(CH₃)— | —CH₂— | 1 | 3 | bond | 2-MeO-phenyl |
| —N(CH₃)— | —CH₂— | 1 | 3 | bond | 3-MeO-phenyl |
| —N(CH₃)— | —CH₂— | 1 | 3 | bond | 3-Cl-phenyl |
| —N(CH₃)— | —CH₂— | 1 | 3 | bond | 2-Cl-phenyl |
| —N(CH₃)— | —CH₂— | 1 | 3 | bond | 3-CF₃-phenyl |
| —N(CH₃)— | —CH₂— | 1 | 2 | bond | 2-Cl-phenyl |
| —N(CH₃)— | —CH₂— | 1 | 2 | bond | 2-MeO-phenyl |
| —N(CH₃)— | —CH₂— | 1 | 3 | bond | 2-Me-phenyl |
| —CH₂— | —CH₂— | 2 | 2 | —C(O)— | 4-F-phenyl |
| —CH₂— | —CH₂— | 2 | 2 | —O— | 4-F-phenyl |
| —CH₂— | —CH₂— | 2 | 2 | bond | 4-F-phenyl |
| —N(CH₃)— | Cyp | 1 | 3 | bond | 2-MeO-phenyl |
| —N(CH₃)— | Cyp | 1 | 2 | bond | benzo[d]isoxazol-3-yl |
| —N(CH₃)— | —CH₂— | 1 | 2 | —C(O)— | 2-Me-4-F-phenyl |
| —O— | —CH₂— | 2 | 2 | —O— | 4-F-phenyl |
| —O— | —CH₂— | 2 | 2 | —C(O)— | 4-F-phenyl |
| —S— | —CH₂— | 2 | 2 | —C(O)— | 4-F-phenyl |
| —CH₂— | —CH₂— | 1 | 2 | —C(O)— | 4-F-phenyl |
| —S— | —CH₂— | 1 | 2 | —C(O)— | 4-F-phenyl |
| —NH— | Cyp | 1 | 2 | —O— | 4-F-phenyl |
| —NH— | Cyp | 1 | 2 | —O— | 2-Me-4-F-phenyl |
| —NH— | Cyp | 1 | 2 | —C(O)— | 4-F-phenyl |
| —NH— | Cyp | 1 | 2 | —C(O)— | 2-Me-4-F-phenyl |
| Cyp | —CH₂— | 1 | 2 | —O— | 4-F-phenyl |
| Cyp | —CH₂— | 1 | 2 | —O— | 2-Me-4-F-phenyl |
| Cyp | —CH₂— | 1 | 2 | —C(O)— | 4-F-phenyl |
| Cyp | —CH₂— | 1 | 2 | —C(O)— | 2-Me-4-F-phenyl |
| —NH— | —C(O)— | 1 | 2 | —C(O)— | 4-F-phenyl |
| —NH— | —C(O)— | 1 | 2 | —C(O)— | 2-Me-4-F-phenyl |
| —NH— | —C(O)— | 1 | 2 | —C(O)— | 2-F-4-Me-phenyl |
| —CH₂— | —CH₂— | 2 | 3 | bond | 2-MeO-phenyl |
| —CH₂— | —CH₂— | 2 | 3 | bond | 2-HO-phenyl |
| —CH₂— | —CH₂— | 2 | 3 | bond | 2-Cl-phenyl |
| —CH₂— | —CH₂— | 2 | 2 | bond | 2-Cl-phenyl |
| —CH₂— | —CH₂— | 2 | 3 | bond | 3-MeO-phenyl |
| —CH₂— | —CH₂— | 2 | 3 | bond | 3-CF₃-phenyl |
| —CH₂— | —CH₂— | 2 | 3 | bond | 3-Cl-phenyl |
| —N(CH₃)— | —CH₂— | 1 | 2 | bond | benzo[d]isoxazol-3-yl |
| —N(CH₃)— | Cyp | 1 | 2 | bond | 6-F-benzo[d]isoxazol-3-yl |
| —N(CH₃)— | —CH₂— | 1 | 2 | bond | 6-F-benzo[d]isoxazol-3-yl |
| Cyp | —CH₂— | 1 | 3 | —O— | 2-Me-4-F-phenyl |
| —N(CH₃)— | —CH₂— | 1 | 3 | —C(O)— | 4-(4-F-PhO)-phenyl |
| Cyp | —CH₂— | 1 | 2 | bond | 2-MeO-phenyl |
| —N(CH₃)— | —CH₂— | 1 | 2 | bond | 3-MeO-phenyl |
| Cyp | —CH₂— | 1 | 2 | bond | benzo[d]isoxazol-3-yl |
| —N(CH₃)— | Cyp | 1 | 4 | bond | 2-CN-phenyl |
| —N(CH₃)— | Cyp | 1 | 2 | bond | 2-CN-phenyl |
| —N(CH₃)— | —CH₂— | 1 | 2 | bond | 2-CN-phenyl |
| —N(CH₃)— | —CH₂— | 1 | 3 | bond | 2-CN-phenyl |
| —N(CH₃)— | —CH₂— | 1 | 2 | bond | 2-oxopyridin-1(2H)-yl |
| —N(CH₃)— | —CH₂— | 1 | 2 | bond | 2-MeO-4-F-phenyl |
| —N(CH₃)— | Cyp | 1 | 2 | bond | 2-MeO-4-F-phenyl |
| —N(CH₃)— | —CH₂— | 1 | 2 | bond | 2-CF₃O-phenyl |
| —N(CH₃)— | —CH₂— | 1 | 2 | bond | 2-Et-phenyl |
| —N(CH₃)— | —CH₂— | 1 | 2 | bond | 4-MeO-phenyl |
| —N(CH₃)— | —CH₂— | 1 | 2 | bond | 3-CN-phenyl |
| —N(CH₃)— | —CH₂— | 1 | 2 | bond | 4-CN-phenyl |
| —N(CH₃)— | —CH₂— | 1 | 2 | bond | 5-F-indol-3-yl |
| —N(CH₃)— | —CH₂— | 1 | 3 | —C(O)— | 2-MeO-phenyl |
| —N(CH₃)— | —C(O)— | 1 | 2 | —O— | 4-F-phenyl |
| —N(CH₃)— | —C(O)— | 1 | 2 | bond | 2-MeO-phenyl |
| —N(CH₃)— | —CH₂— | 1 | 2 | bond | 2-HO-phenyl |
| —N(CH₃)— | —CH₂— | 1 | 1 | bond | 2-MeO-phenyl |
| —N(CH₃)— | —CH₂— | 1 | 2 | bond | 2-EtO-phenyl |
| —N(CH₃)— | —CH₂— | 1 | 2 | bond | benzofuran-7-yl |
| —N(CH₃)— | —CH₂— | 1 | 3 | —O— | 2-MeO-phenyl |
| —N(CH₃)— | —CH₂— | 1 | 2 | bond | benzo[d]isothiazol-3-yl |
| —N(CH₃)— | —CH₂— | 1 | 2 | bond | 2.5-di-MeO-phenyl |
| —N(CH₃)— | —CH₂— | 1 | 1 | bond | 4-Et-phenyl |
| —N(CH₃)— | —CH₂— | 1 | 2 | bond | indol-3-y1 |
| —N(CH₃)— | Cyp | 1 | 2 | bond | 2-Et-phenyl |
| —N(CH₃)— | Cyp | 1 | 2 | bond | 3-MeO-phenyl |
| —N(CH₃)— | —CH₂— | 1 | 1 | bond | phenyl |
| —N(CH₃)— | —CH₂— | 1 | 1 | bond | 3-MeO-phenyl |
| —N(CH₃)— | —CH₂— | 1 | 1 | bond | 3-Et-phenyl |
| —N(CH₃)— | —CH₂— | 1 | 1 | bond | 4-MeO-phenyl |
| —N(CH₃)— | —CH₂— | 1 | 1 | bond | 2-Et-phenyl |
| —N⁺(═O)⁻— | Cyp | 1 | 2 | —O— | 4-F-phenyl |
| —N(CH₃)— | —CH₂— | 1 | 2 | bond | 2-MeSO₂-phenyl |
| —N(CH₃)— | Cyp | 1 | 2 | bond | benzo[d]isothiazol-3-yl |
| —N(CH₃)— | —CH₂— | 1 | 1 | bond | cyclohexyl |
| —N(CH₃)— | —CH₂— | 1 | 2 | bond | 2-MeO-5-F-phenyl |
| —N(CH₃)— | —CH₂— | 1 | 2 | bond | 3-Et-Phenyl |
| —N(CH₃)— | —CH₂— | 1 | 2 | bond | 3-EtO-phenyl |
| —N(CH₃)— | Cyp | 1 | 2 | bond | benzofuran-7-yl |
| —N(CH₃)— | —CH₂— | 1 | 2 | bond | benzo[d]imidazole-1-yl |
| —N(CH₃)— | —CH₂— | 1 | 2 | bond | benzo[d][1,2,3]triazol-1-yl |
| —N(CH₃)— | —CH₂— | 1 | 2 | bond | indazol-3-yl |
| —NH— | —C(O)— | 1 | 2 | bond | benzo[d]isothiazol-3-yl |
| —N(CH₃)— | —C(O)— | 1 | 2 | bond | benzo[d]isothiazol-3-yl |
| —N(CH₃)— | Cyp | 1 | 1 | bond | 2-MeO-phenyl |
| —N(CH₃)— | —CH₂— | 1 | 1 | bond | benzo[d]isoxazol-3-yl |
| —N(CH₃)— | —CH₂— | 1 | 1 | bond | benzofuran-7-yl |
| —N(CH₃)— | —CH₂— | 1 | 1 | bond | benzo[d]isothiazol-3-yl |
| —N(CH₃)— | —CH₂— | 1 | 1 | bond | quinolin-8-yl |
| —N(CH₃)— | —CH₂— | 1 | 1 | bond | benzo[d]oxazol-7-yl |
| —N(CH₃)— | —CH₂— | 1 | 1 | bond | 2.3-dihydrobenzofuran-7-yl |
| —N(CH₃)— | —CH₂— | 1 | 2 | —O— | 2-CN-4-F-phenyl |
| —N(CH₃)— | —CH₂— | 1 | 2 | bond | indazol-1-yl |
| —N(CH₃)— | —C(O)— | 1 | 2 | —C(O)— | 4-F-phenyl |
| —N(CH₃)— | —CH₂— | 1 | 1 | bond | quinoxalin-5-yl |
| —N(CH₃)— | —CH₂— | 1 | 1 | —C(O)— | 4-F-Phenyl |
| —N(CH₃)— | —CH₂— | 1 | 2 | —C(═NOMe) | 4-F-Ph |
| —N(CH₃)— | —CH₂— | 1 | 3 | —C(O)— | phenyl |
| —N(CH₃)— | —CH₂— | 1 | 2 | —C(═NOMe) | phenyl |
| —N(CH₃)— | —C(O)— | 1 | 2 | —C(O)— | phenyl |
| —N(CH₃)— | —CH₂— | 1 | 2 | —C(O)— | thiophen-2-yl |
| —N(CH₃)— | —CH₂— | 1 | 1 | bond | 3-(O-cyclopropyl)phenyl |
| —N(CH₃)— | —CH₂— | 1 | 1 | bond | 3-(OCH₂-cyclopropyl)phenyl |
| —N(CH₃)— | —C(O)— | 1 | 2 | bond | 2-MeO-phenyl |
| —N(CH₃)— | —C(O)— | 1 | 2 | bond | 3-MeO-phenyl |
| —N(CH₃)— | —C(O)— | 1 | 2 | bond | benzo[d]isoxazol-3-yl |
| —N(CH₃)— | —CH₂— | 1 | 1 | bond | 3-MeS-phenyl |
| —N(CH₃)— | Cyp | 1 | 2 | —C(O)— | phenyl |
| —N(CH₃)— | Cyp | 1 | 3 | —C(OH)— | 4-F-phenyl |
| —N(CH₃)— | Cyp | 1 | 3 | —C(OH)— | phenyl |
| —N(CH₃)— | Cyp | 1 | 3 | —C(OMe)— | 4-F-phenyl |
| —N(CH₃)— | Cyp | 1 | 3 | —C(OMe)— | phenyl |
| —N(CH₃)— | —CH₂— | 1 | 2 | bond | 2-(MeNH)-phenyl |
| —N(CH₃)— | Cyp | 1 | 2 | bond | 2-HO-phenyl |
| —N(CH₃)— | Cyp | 1 | 2 | bond | 2-MeO-phenyl |
| —N(CH₃)— | Cyp | 1 | 2 | bond | 2-EtO-phenyl |
| —N(CH₃)— | Cyp | 1 | 2 | bond | 2-CF₃O-phenyl |
| —N(CH₃)— | Cyp | 1 | 2 | bond | 2-MeSO₂-phenyl |
| —N(CH₃)— | Cyp | 1 | 2 | bond | 2-(MeNH)-phenyl |
| —N(CH₃)— | Cyp | 1 | 2 | bond | 3-CN-phenyl |
| —N(CH₃)— | Cyp | 1 | 2 | bond | 4-MeO-phenyl |
| —N(CH₃)— | Cyp | 1 | 2 | bond | 4-CN-phenyl |
| —N(CH₃)— | —CH₂— | 1 | 1 | bond | quinazolin-7-yl |
| —N(CH₃)— | —CH₂— | 1 | 1 | bond | isoquinolin-7-yl |
| —N(CH₃)— | —CH₂— | 1 | 1 | bond | benzo[d]isoxazol-4-yl |
| —N(CH₃)— | —CH₂— | 1 | 1 | bond | benzofuran-4-yl |
| —N(CH₃)— | —CH₂— | 1 | 1 | bond | 2-Me-benzofuran-7-yl |
| —N(CH₃)— | —CH₂— | 1 | 2 | bond | 3-MeS-phenyl |
| —N(CH₃)— | —CH₂— | 1 | 2 | bond | 3-(O-Cyclopropyl)phenyl |
| —N(CH₃)— | —CH₂— | 1 | 2 | bond | 3-(OCH₂-Cyclopropyl)phenyl |
| —N(CH₃)— | —CH₂— | 1 | 2 | bond | benzo[d]isoxazol-4-yl |
| —N(CH₃)— | —CH₂— | 1 | 2 | bond | benzofuran-4-yl |
| —N(CH₃)— | —CH₂— | 1 | 2 | bond | 2-Me-benzofuran-7-yl |
| —N(CH₃)— | Cyp | 1 | 2 | bond | indazol-7-yl |
| —N(CH₃)— | Cyp | 1 | 2 | bond | quinolin-8-yl |
| —N(CH₃)— | Cyp | 1 | 2 | bond | pyrid-4-y1 |
| —N(CH₃)— | —CH₂— | 1 | 2 | bond | quinolin-8-yl |
| —N(CH₃)— | —CH₂— | 1 | 2 | bond | indol-1-yl |
| —N(CH₃)— | —CH₂— | 1 | 2 | bond | pyrid-4-yl |
| —N(CH₃)— | Cyp | 1 | 1 | bond | 3-Et-phenyl |
| —N(CH₃)— | Cyp | 1 | 2 | —O— | 3-CN-4-F-phenyl |
| —N(CH₃)— | —CH₂— | 1 | 1 | bond | H |
| —N(CH₃)— | —CH₂— | 1 | 2 | bond | H |
| —N(CH₃)— | —CH₂— | 1 | 3 | bond | H |
| —N(CH₃)— | —CH₂— | 1 | 1 | bond | Cyclopentyl |
| —N(CH₃)— | —CH₂— | 1 | 1 | bond | Cyclobutyl |
| —N(CH₃)— | —CH₂— | 1 | 1 | bond | Cyclopropyl |

each independently in free or pharmaceutically acceptable salt or form, wherein Cyp refers to a spiro-joined cyclopropyl ring.
